(19) United States
(12) Patent Application Publication
(10) Pub. No.: US 2008/0194590 A1
(43) Pub. Date: Aug. 14, 2008

(54) 4-Piperazinylthieno [2,3-D] Pyrimidine Compounds as Platelet Aggregation Inhibitors

(75) Inventors: Michael Dalton Ennis, Chesterfield, MO (US); Steven Wade Kortum, Chesterfield, MO (US); Ruth Elizabeth Tenbrink, Chesterfield, MO (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 11/908,815

(22) PCT Filed: Mar. 20, 2006
(86) PCT No.: PCT/IB06/00737

§ 371 Date: Sep. 17, 2007

Related U.S. Application Data
(60) Provisional application No. 60/665,732, filed on Mar. 28, 2005.

(51) Int. Cl. A61K 31/4985; C07D 519/00; A61P 7/02

(52) U.S. Cl. 514/260.1; 544/278

(57) ABSTRACT

Compounds and pharmaceutically acceptable salts of the compounds are disclosed, wherein the compounds have the structure of Formula (I) wherein A¹, A², A³, A⁴, A⁵, A⁶, A⁷, A⁸, X⁴, X⁶, X^{2k}, R^{2l}, R⁴, R⁵, and R⁶ are as defined in the detailed description of the invention. Corresponding pharmaceutical compositions, methods of treatment, methods of synthesis, and intermediates are also disclosed.

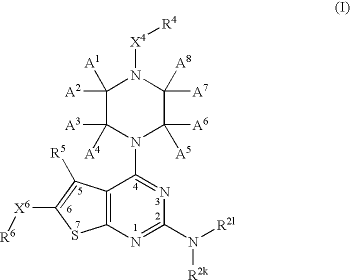

### CROSS REFERENCE TO OTHER APPLICATIONS

This application claims priority to U.S. Provisional application No. 60/665,732, filed Mar. 28, 2005.

### FIELD OF THE INVENTION

The present invention comprises a novel class of thieno[2,3-d]pyrimidine compounds having the structure of Formula I (including tautomers and salts of those compounds) and pharmaceutical compositions comprising a compound of Formula I. The present invention also comprises methods of treating a subject by administering a therapeutically effective amount of a compound of Formula I to the subject. In general, these compounds, in whole or in part, inhibit ADP-mediated platelet aggregation. The present invention further comprises methods for making the compounds of Formula I and corresponding intermediates.

### BACKGROUND OF THE INVENTION

Thrombosis is a pathological process in which a platelet aggregate and/or a fibrin clot occludes a blood vessel. Arterial thrombosis may result in ischemic necrosis of the tissue supplied by the artery. Venous thrombosis may cause edema and inflammation in the tissue drained by the vein. Compounds that inhibit platelet function can be administered to a patient to decrease the risk of occlusive arterial events in patients suffering from or susceptible to atherosclerotic cardiovascular, cerebrovascular and peripheral arterial diseases. Commercially available drugs that inhibit platelet function typically fall within one of three classes of drugs that antagonize different molecular targets: (1) cycloxygenase inhibitors, such as aspirin (see Awtry, E. H. et al., Circulation, 2000, Vol. 101, pg. 1206); (2) glycoprotein IIb-IIIa antagonists, such as tirofiban (see Scarborough, R. M. et al., Journal of Medicinal Chemistry, 2000, Vol. 43, pg. 3453); and (3) P2Y12 receptor antagonists (also known as ADP receptor antagonists), such as the thienopyridine compounds ticlopidine and clopidogrel (see Quinn, M. J. et al., Circulation, 1999, Vol. 100, pg. 1667.

There are several disadvantages associated with use of the P2Y12 receptor antagonists ticlopidine and clopidogrel. First, although both compounds selectively inhibit platelet aggregation by blocking the P2Y12 receptor, such inhibition is irreversible and increases the bleeding risk to the patient. Second, both ticlopidine and clopidogrel each have a relatively slow onset of action. Both compounds apparently are prodrugs that first must be metabolized by the liver into the corresponding active metabolites. Third, a number of patients are resistant to treatment with clopidogrel. Such resistance may result, in whole or in part, from drug-drug interactions between clopidogrel and other drugs commonly administered to atherosclerotic patients. Fourth, both ticlopidine and clopidogrel have been associated with side-effects such as thrombocytopenia in some patients (see Bennett, C. L. et al., New England Journal of Medicine, 2000, Vol. 342, pg. 1773).

Other compounds have been reported in the literature as useful for the treatment of cardiovascular events such as thrombosis:

US2003/0153566 A1 (published Aug. 14, 2003) describes a class of piperazine compounds as ADP receptor antagonists.

WIPO Int'l Publ. No. WO99/05144 A1 (published Feb. 4, 1999) describes a class of triazolo[4,5-d]pyrimidine compounds as P2T antagonists.

WIPO Int'l Publ. No. WO99/36425 A1 (published Jul. 22, 1999) describes a class of tricyclic compounds as ADP receptor antagonists.

WIPO Int'l Publ. No. WO01/57037 A1 (published Aug. 9, 2001) describes a class of compounds including sulfonylureas as ADP receptor antagonists.

U.S. Pat. No. 5,057,517 (granted Oct. 15, 1991) describes a class of heteroaromatic compounds including 6-piperazinopurines as antidiabetic agents.

U.S. Pat. No. 4,459,296 (granted Jul. 10, 1984) describes a class of N-(benzimidazolyl, indolyl, purinyl or benzotriazolyl)-piperazine compounds as antihypertensive agents.

Humphries et al. describe several purine compounds as selective ADP receptor antagonists in an animal thrombosis model. Trends in Pharmacological Sciences, 1995, Vol. 16, pg. 179. These compounds are further described in Ingall, A. H et al., Journal of Medicinal Chemistry, 1999, Vol. 42, pg. 213.

Accordingly, a need still exists for new drug therapies for the treatment of subjects suffering from or susceptible to a platelet aggregation mediated condition. In particular, a need still exists for new P2Y12 antagonists having one or more improved properties (such as safety profile, efficacy, or physical properties) relative to currently available P2Y12 antagonists.

### SUMMARY OF THE INVENTION

In one embodiment, the invention comprises a class of compounds (including the pharmaceutically acceptable salts of the compounds) having the structure of Formula I:

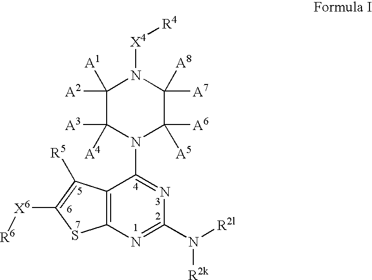

wherein A¹, A², A³, A⁴, A⁵, A⁶, A⁷, A⁸, X⁴, X⁶, R^{2k}, R^{2l}, R⁴, R⁵, and R⁶ are as defined in the detailed description of the invention.

In another embodiment, the invention comprises a pharmaceutical composition comprising a compound having the structure of Formula I.

In another embodiment, the invention comprises methods of treating a condition in a subject by administering to a subject a therapeutically effective amount of a compound having the structure of Formula I. The conditions that can be treated in accordance with the present invention include, but are not limited to, atherosclerotic cardiovascular diseases, cerebrovascular diseases and peripheral arterial diseases. Other conditions that can be treated in accordance with the present invention include hypertension and angiogenesis.

In another embodiment, the invention comprises methods for inhibiting platelet aggregation in a subject by administering to the subject a compound having a structure of Formula I.

In another embodiment, the invention comprises methods of making compounds having the structure of Formula I.

In another embodiment, the invention comprises intermediates useful in the synthesis of compounds having the structure of Formula I.

### DETAILED DESCRIPTION OF THE INVENTION

This detailed description of embodiments is intended only to acquaint others skilled in the art with Applicants' inventions, its principles, and its practical application so that others skilled in the art may adapt and apply the inventions in their numerous forms, as they may be best suited to the requirements of a particular use. These inventions, therefore, are not limited to the embodiments described in this specification, and may be variously modified.

### A. Abbreviations and Definitions

TABLE A
Abbreviations

| | |
|---|---|
| 1-HOAT | 1-hydroxy-7-azabenzotriazole |
| 1-HOBt | 1-hydroxybenzotriazole hydrate |
| ADP | Adenosine diphosphate (the natural ligand of P2Y12) |
| AMP | Adenosine monophospate |
| ASA | Acetylsalicylic acid |
| ATP | Adenosine triphosphate |
| Bn | Benzyl group |
| Boc | tert-butoxycarbonyl |
| BOP-Cl | bis(2-oxo-3-oxazolidinyl)phosphinic chloride |
| br | Broad |
| BSA | Bovine serum albumin |
| Cbz | Benzyloxycarbonyl |
| CD₃OD | Deuterated methanol |
| CDCl₃ | Deuterated chloroform |
| CDI | 1,1′-carbonyldiimidazole |
| d | Doublet |
| DBN | 1,5-diazabicyclo[4.3.0]non-5-ene |
| DBU | 1,8-diazabicyclo[5.4.0]undec-7-ene |
| DCC | 1,3-dicyclohexylcarbodiimide |
| DCM | Dichloromethane |
| dd | Doublet of doublets |
| DEPC | diethyl cyanophosphonate |
| DIEA | Diisopropylethylamine |
| DMF | N,N-dimethylformamide |
| DMSO | dimethyl sulphoxide |
| DPBS | Dulbecco's Phosphate Buffered Saline |
| EBSS | Earle's Balanced Salt Solution |
| EDC | 1-(3-dimethylaminopropyl)-3-ethylcarbodiimide |
| | hydrochloride |
| EDTA | ethylenediaminetetraacetic acid |
| EGTA | ethyleneglycol-bis(β-aminoethyl)-N,N,N′,N′- |
| | tetraacetic Acid |
| ESI | Electrospray Ionization for mass spectrometry |
| Et₃N | Triethylamine |
| EtOAc | ethyl acetate |
| EtOH | Ethanol |
| FBS | Fetal bovine serum |
| Fmoc | Fluorene methyloxycarbonyl |
| HATU | O-(7-azabenzotriazol-1-yl)-N,N,N′,N′-tetramethyluronium |
| | hexafluorophosphate |
| HBTU | O-benzotriazol-1-yl-N,N,N′,N′-tetramethyluronium |
| | hexafluorophosphate |
| HCl | Hydrochloric acid |
| HEK | Human embryonic kidney |
| HEPES | 4-(2-hydroxyethyl)-1-Piperazineethane sulfonic acid |
| HRMS | High Resolution Mass Spectroscopy (electrospray |
| | ionization positive scan) |
| K₃PO₄ | Potassium phosphate |
| LCMS | Liquid Chromatography-Mass Spectroscopy |
| LRMS | Low Resolution Mass Spectroscopy (electrospray or |
| | thermospray ionization positive scan) |
| LRMS (ES⁻) | Low Resolution Mass Spectroscopy (electrospray |
| | ionization negative scan) |
| m | Multiplet |
| m/z | Mass spectrum peak |
| MEM | Minimum essential medium |
| MeOH | Methanol |
| MHz | Megahertz |
| MS | Mass spectroscopy |
| NaH | Sodium hydride |
| NMM | N-methylmorpholine |
| NMP | 1-methyl-2-pyrrolidinone |
| NMR | Nuclear Magnetic Resonance |
| PG | Protecting group. Exemplary protecting groups include |
| | Boc, Cbz, Fmoc and benzyl |
| Pg. | Page |
| PPP | Platelet poor plasma |
| PRP | Platelet rich plasma |
| q | Quartet |
| Rpm | Revolutions per minute |
| s | Singlet |
| t | Triplet |
| TFA | trifluoroacetic acid |
| THF | Tetrahydrofuran |
| TLC | Thin layer chromatography |
| Vol. | Volume |
| δ | Chemical shift |

The term “alkyl” refers to a linear or branched-chain saturated hydrocarbyl substituent (i.e., a substituent containing only carbon and hydrogen) containing in one embodiment, from about one to about twenty carbon atoms; in another embodiment from about one to about twelve carbon atoms; in another embodiment, from about one to about ten carbon atoms; in another embodiment, from about one to about six carbon atoms; and in another embodiment, from about one to about four carbon atoms. Examples of such substituents include methyl, ethyl, propyl (including n-propyl and isopropyl), butyl (including n-butyl, isobutyl, sec-butyl and tert-butyl), pentyl, iso-amyl, hexyl and the like.

The term “alkenyl” refers to a linear or branched-chain hydrocarbyl substituent containing one or more double bonds and from about two to about twenty carbon atoms; in another embodiment, from about two to about twelve carbon atoms; in another embodiment, from about two to about six carbon atoms; and in another embodiment, from about two to about four carbon atoms. Examples of alkenyl include ethenyl (also known as vinyl), allyl, propenyl (including 1-propenyl and 2-propenyl) and butenyl (including 1-butenyl, 2-butenyl and 3-butenyl). The term “alkeny” embraces substituents having “cis” and “trans” orientations, or alternatively, “E” and “Z” orientations.

The term “alkynyl” refers to linear or branched-chain hydrocarbyl substituents containing one or more triple bonds and from about two to about twenty carbon atoms; in another embodiment, from about two to about twelve carbon atoms; in another embodiment, from about two to about six carbon atoms; and in another embodiment, from about two to about four carbon atoms. Examples of alkynyl substituents include ethynyl, propynyl (including 1-propynyl and 2-propynyl) and butynyl (including 1-butynyl, 2-butynyl and 3-butynyl).

The term “benzyl” refers to methyl radical substituted with phenyl, i.e., the following structure:

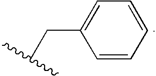

The term “carbocyclyl” refers to a saturated cyclic (i.e., “cycloalkyl”), partially saturated cyclic (i.e., “cycloalkenyl”), or completely unsaturated (i.e., “aryl”) hydrocarbyl substituent containing from 3 to 14 carbon ring atoms (“ring atoms” are the atoms bound together to form the ring or rings of a cyclic substituent). A carbocyclyl may be a single ring, which typically contains from 3 to 6 ring atoms. Examples of such single-ring carbocyclyls include cyclopropyl, cyclobutyl, cyclopentyl, cyclopentenyl, cyclopentadienyl, cyclohexyl, cyclohexenyl, cyclohexadienyl, and phenyl. A carbocyclyl alternatively may be 2 or 3 rings fused together, such as naphthalenyl, tetrahydronaphthalenyl (also known as “tetralinyl”), indenyl, isoindenyl, indanyl, bicyclodecanyl, anthracenyl, phenanthrene, benzonaphthenyl (also known as “phenalenyl”), fluorenyl, and decalinyl.

The term “cycloalkyl” refers to a saturated carbocyclic substituent having three to about fourteen carbon atoms. In another embodiment, a cycloalkyl substituent has three to about eight carbon atoms. Examples of cycloalkyl include cyclopropyl, cyclobutyl, cyclopentyl and cyclohexyl. The term “cycloalkylalkyl” refers to alkyl substituted with cycloalkyl. Examples of cycloalkylalkyl include cyclopropylmethyl, cyclobutylmethyl, cyclopentylmethyl, and cyclohexylmethyl. The term “cycloalkenyl” refers to a partially unsaturated carbocyclyl substituent. Examples of cycloalkenyl include cyclobutenyl, cyclopentenyl, and cyclohexenyl.

The term “aryl” refers to a carbocyclic aromatic system containing one, two or three rings wherein such rings may be attached together in a pendent manner or may be fused. The term “aryl” refers to aromatic substituents such as phenyl, naphthyl and anthracenyl.

The term “arylalkyl” refers to alkyl substituted with aryl.

In some instances, the number of carbon atoms in a hydrocarbyl substituent (e.g., alkyl, alkenyl, alkynyl, cycloalkyl, cycloalkenyl, aryl, etc.) is indicated by the prefix “Cₓ-C_{y}-,” wherein x is the minimum and y is the maximum number of carbon atoms in the substituent. Thus, for example, “C₁-C₆-alkyl” refers to an alkyl substituent containing from 1 to 6 carbon atoms. Illustrating further, C₃-C₆-cycloalkyl refers to saturated carbocyclyl containing from 3 to 6 carbon ring atoms. The term “hydrogen” refers to hydrogen substituent, and may be depicted as —H.

The term “hydroxy” refers to —OH. When used in combination with another term(s), the prefix “hydroxy” indicates that the substituent to which the prefix is attached is substituted with one or more hydroxy substituents. Compounds bearing a carbon to which one or more hydroxy substituents include, for example, alcohols, enols and phenol.

The term “hydroxyalkyl” refers to an alkyl that is substituted with at least one hydroxy substituent. Examples of hydroxyalkyl include hydroxymethyl, hydroxyethyl, hydroxypropyl and hydroxybutyl. The term “nitro” means —NO₂.

The term “cyano” (also referred to as “nitrile”) —CN, which also may be depicted:

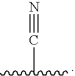

The term “carbonyl” refers to —C(O)—, which also may be depicted as:

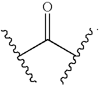

The term “amino” refers to —NH₂.

The term “alkylamino” refers to an amino group, wherein at least one alkyl chain is bonded to the amino nitrogen in place of a hydrogen atom. Examples of alkylamino substituents include monoalkylamino such as methylamino (exemplified by the formula —NH(CH₃)), which may also be depicted:

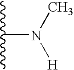

and dialkylamino such as dimethylamino, (exemplified by the formula —N((CH₃)₂), which may also be depicted:

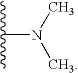

The term “aminocarbonyl” refers to —C(O)—NH₂, which also may be depicted as:

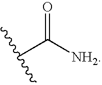

The term “halogen” refers to fluorine (which may be depicted as —F), chlorine (which may be depicted as —Cl), bromine (which may be depicted as —Br), or iodine (which may be depicted as —I). In one embodiment, the halogen is chlorine. In another embodiment, the halogen is a fluorine.

The prefix “halo” indicates that the substituent to which the prefix is attached is substituted with one or more independently selected halogen substituents. For example, haloalkyl refers to an alkyl that is substituted with at least one halogen substituent. Where there is more than one hydrogen replaced with halogens, the halogens may be the identical or different. Examples of haloalkyls include chloromethyl, dichloromethyl, difluorochloromethyl, dichlorofluoromethyl, trichloromethyl, 1-bromoethyl, fluoromethyl, difluoromethyl, trifluoromethyl, 2,2,2-trifluoroethyl, difluoroethyl, pentafluoroethyl, difluoropropyl, dichloropropyl, and heptafluoropropyl. Illustrating further, “haloalkoxy” refers to an alkoxy that is substituted with at least one halogen substituent. Examples of haloalkoxy substituents include chloromethoxy, 1-bromoethoxy, fluoromethoxy, difluoromethoxy, trifluoromethoxy (also known as “perfluoromethyloxy”), and 2,2,2-trifluoroethoxy. It should be recognized that if a substituent is substituted by more than one halogen substituent, those halogen substituents may be identical or different (unless otherwise stated).

The prefix “perhalo” indicates that each hydrogen substituent on the substituent to which the prefix is attached is replaced with an independently selected halogen substituent. If all the halogen substituents are identical, the prefix may identify the halogen substituent. Thus, for example, the term “perfluoro” means that every hydrogen substituent on the substituent to which the prefix is attached is replaced with a fluorine substituent. To illustrate, the term “perfluoroalkyl” refers to an alkyl substituent wherein a fluorine substituent is in the place of each hydrogen substituent. Examples of perfluoroalkyl substituents include trifluoromethyl (—CF₃), perfluorobutyl, perfluoroisopropyl, perfluorododecyl, and perfluorodecyl. To illustrate further, the term “perfluoroalkoxy” refers to an alkoxy substituent wherein each hydrogen substituent is replaced with a fluorine substituent. Examples of perfluoroalkoxy substituents include trifluoromethoxy (—O—CF₃), perfluorobutoxy, perfluoroisopropoxy, perfluorododecoxy, and perfluorodecoxy.

The term “oxo” refers to ═O.

The term “oxy” refers to an ether substituent, and may be depicted as —O—.

The term “alkoxy” refers to an alkyl linked to an oxygen, which may also be represented as —O—R, wherein the R represents the alkyl group. Examples of alkoxy include methoxy, ethoxy, propoxy and butoxy.

The term “alkylthio” refers to —S-alkyl. For example, “methylthio” is —S—CH₃. Other examples of alkylthio include ethylthio, propylthio, butylthio, and hexylthio.

The term “alkylcarbonyl” refers to —C(O)-alkyl. For example, “ethylcarbonyl” may be depicted as:

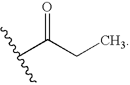

Examples of other alkylcarbonyl include methylcarbonyl, propylcarbonyl, butylcarbonyl, pentylcarbonyl, and hexylcarbonyl.

The term “aminoalkylcarbonyl” refers to —C(O)-alkyl-NH₂. For example, “aminomethylcarbonyl” may be depicted as:

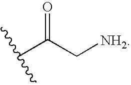

The term “alkoxycarbonyl” refers to —C(O)-O-alkyl. For example, “ethoxycarbonyl” may be depicted as:

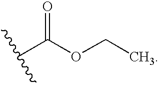

Examples of other alkoxycarbonyl include methoxycarbonyl, ethoxycarbonyl, propoxycarbonyl, butoxycarbonyl, pentoxycarbonyl, and hexyloxycarbonyl. In another embodiment, where the carbon atom of the carbonyl is attached to a carbon atom of a second alkyl, the resulting functional group is an ester.

The term “carbocyclylcarbonyl” refers to —C(O)-carbocyclyl. For example, “phenylcarbonyl” may be depicted as:

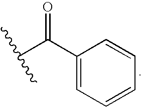

Similarly, the term “heterocyclylcarbonyl,” alone or in combination with another term(s), refers to —C(O)-heterocyclyl.

The term “carbocyclylalkylcarbonyl” refers to —C(O)-alkyl-carbocyclyl. For example, “phenylethylcarbonyl” may be depicted as:

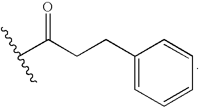

Similarly, the term “heterocyclylalkylcarbonyl,” alone or in combination with another term(s), means —C(O)-alkyl-heterocyclyl.

The term “carbocyclyloxycarbonyl,” refers to —C(O)—O-carbocyclyl. For example, “phenyloxycarbonyl” may be depicted as:

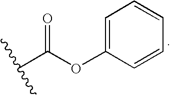

The term “carbocyclylalkoxycarbonyl” refers to —C(O)—O-alkyl-carbocyclyl. For example, “phenylethoxycarbonyl” may be depicted as:

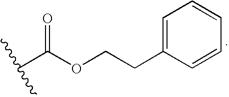

The terms “thio” and “thia” refer to a divalent sulfur atom and such a substituent may be depicted as —S—. For example, a thioether is represented as “alkyl-thio-alkyl” or, alternatively, alkyl-S-alkyl. The term “thiol” refers to a sulfhydryl substituent, and may be depicted as —SH. The term “thione” refers to ═S.

The term “sulfonyl” refers to —S(O)₂—, which also may be depicted as:

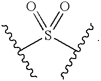

Thus, for example, “alkyl-sulfonyl-alkyl” refers to alkyl-S(O)₂-alkyl. Examples of alkylsulfonyl include methylsulfonyl, ethylsulfonyl, and propylsulfonyl.

The term “aminosulfonyl” refers to —S(O)₂—NH₂, which also may be depicted as:

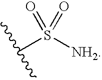

The terms “sulfinyl” and “sulfoxido” refer to —S(O)—, which also may be depicted as:

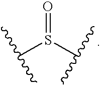

Thus, for example, “alkylsulfinylalkyl” or “alkylsulfoxidoalkyl” refers to alkyl-S(O)-alkyl. Exemplary alkylsulfinyl groups include methylsulfinyl, ethylsulfinyl, butylsulfinyl, and hexylsulfinyl.

The term “heterocyclyl” refers to a saturated, partially saturated, or completely unsaturated ring structure containing a total of 3 to 14 ring atoms. At least one of the ring atoms is a heteroatom (i.e., oxygen, nitrogen, or sulfur), with the remaining ring atoms being independently selected from the group consisting of carbon, oxygen, nitrogen, and sulfur.

A heterocyclyl may be a single ring, which typically contains from 3 to 7 ring atoms, more typically from 3 to 6 ring atoms, and even more typically 5 to 6 ring atoms. Examples of single-ring heterocyclyls include furanyl, dihydrofurnayl, tetradydofurnayl, thiophenyl (also known as “thiofuranyl”), dihydrothiophenyl, tetrahydrothiophenyl, pyrrolyl, isopyrrolyl, pyrrolinyl, pyrrolidinyl, imidazolyl, isoimidazolyl, imidazolinyl, imidazolidinyl, pyrazolyl, pyrazolinyl, pyrazolidinyl, triazolyl, tetrazolyl, dithiolyl, oxathiolyl, oxazolyl, isoxazolyl, thiazolyl, isothiazolyl, thiazolinyl, isothiazolinyl, thiazolidinyl, isothiazolidinyl, thiodiazolyl, oxathiazolyl, oxadiazolyl (including oxadiazolyl, 1,2,4-oxadiazolyl (also known as “azoximyl”), 1,2,5-oxadiazolyl (also known as “furazanyl”), or 1,3,4-oxadiazolyl), oxatriazolyl (including 1,2,3,4-oxatriazolyl or 1,2,3,5-oxatriazolyl), dioxazolyl (including 1,2,3-dioxazolyl, 1,2,4-dioxazolyl, 1,3,2-dioxazolyl, or 1,3,4-dioxazolyl), oxathiazolyl, oxathiolyl, oxathiolanyl, pyranyl (including 1,2-pyranyl or 1,4-pyranyl), dihydropyranyl, pyridinyl (also known as “azinyl”), piperidinyl, diazinyl (including pyridazinyl (also known as “1,2-diazinyl”), pyrimidinyl (also known as “1,3-diazinyl” or “pyrimidyl”), or pyrazinyl (also known as “1,4-diazinyl”)), piperazinyl, triazinyl (including s-triazinyl (also known as “1,3,5-triazinyl”), as-triazinyl (also known 1,2,4-triazinyl), and v-triazinyl (also known as “1,2,3-triazinyl”)), oxazinyl (including 1,2,3-oxazinyl, 1,3,2-oxazinyl, 1,3,6-oxazinyl (also known as “pentoxazolyl”), 1,2,6-oxazinyl, or 1,4-oxazinyl), isoxazinyl (including o-isoxazinyl or p-isoxazinyl), oxazolidinyl, isoxazolidinyl, oxathiazinyl (including 1,2,5-oxathiazinyl or 1,2,6-oxathiazinyl), oxadiazinyl (including 1,4,2-oxadiazinyl or 1,3,5,2-oxadiazinyl), morpholinyl, azepinyl, oxepinyl, thiepinyl, and diazepinyl.

A heterocyclyl alternatively may comprise 2 or 3 rings fused together, wherein at least one such ring contains a heteroatom as a ring atom (e.g., nitrogen, oxygen, or sulfur). Examples of 2-fused-ring heterocyclyls include, indolizinyl, pyrindinyl, pyranopyrrolyl, 4H-quinolizinyl, purinyl, naphthyridinyl, pyridopyridinyl (including pyrido[3,4-b]-pyridinyl, pyrido[3,2-b]-pyridinyl, or pyrido[4,3-b]-pyridinyl), and pteridinyl, indolyl, isoindolyl, indoleninyl, isoindazolyl, benzazinyl, phthalazinyl, quinoxalinyl, quinazolinyl, benzodiazinyl, benzopyranyl, benzothiopyranyl, benzoxazolyl, indoxazinyl, anthranilyl, benzodioxolyl, benzodioxanyl, benzoxadiazolyl, benzofuranyl, isobenzofuranyl, benzothienyl, isobenzothienyl, benzothiazolyl, benzothiadiazolyl, benzimidazolyl, benzotriazolyl, benzoxazinyl, benzisoxazinyl, and tetrahydroisoquinolinyl. Other examples of fused-ring heterocyclyls include benzo-fused heterocyclyls, such as indolyl, isoindolyl (also known as “isobenzazolyl” or “pseudoisoindolyl”), indoleninyl (also known as “pseudoindolyl”), isoindazolyl (also known as “benzpyrazolyl”), benzazinyl (including quinolinyl (also known as “1-benzazinyl”) or isoquinolinyl (also known as “2-benzazinyl”)), phthalazinyl, quinoxalinyl, quinazolinyl, benzodiazinyl (including cinnolinyl (also known as “1,2-benzodiazinyl”) or quinazolinyl (also known as “1,3-benzodiazinyl”)), benzopyranyl (including “chromanyl” or “isochromanyl”), benzothiopyranyl (also known as “thiochromanyl”), benzoxazolyl, indoxazinyl (also known as “benzisoxazolyl”), anthranilyl, benzodioxolyl, benzodioxanyl, benzoxadiazolyl, benzofuranyl (also known as “coumaronyl”), isobenzofuranyl, benzothienyl (also known as “benzothiophenyl,” “thionaphthenyl,” or “benzothiofuranyl”), isobenzothienyl (also known as “isobenzothiophenyl,” “isothionaphthenyl,” or “isobenzothiofuranyl”), benzothiazolyl, benzothiadiazolyl, benzimidazolyl, benzotriazolyl, benzoxazinyl (including 1,3,2-benzoxazinyl, 1,4,2-benzoxazinyl, 2,3,1-benzoxazinyl, or 3,1,4-benzoxazinyl ), benzisoxazinyl (including 1,2-benzisoxazinyl or 1,4-benzisoxazinyl), tetrahydroisoquinolinyl, carbazolyl, xanthenyl, and acridinyl.

The term “heteroaryl” refers to an aromatic heterocyclyl containing from 5 to 14 ring atoms. A heteroaryl may be a single ring or 2 or 3 fused rings. Examples of heteroaryl substituents include 6-membered ring substituents such as pyridyl, pyrazyl, pyrimidinyl, and pyridazinyl; 5-membered ring substituents such as triazolyl, imidazyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl; 6/5-membered fused ring substituents such as benzothiofuranyl, isobenzothiofuranyl, benzisoxazolyl, benzoxazolyl, purinyl, and anthranilyl; and 6/6-membered fused rings such as quinolinyl, isoquinolinyl, cinnolinyl, quinazolinyl, and 1,4-benzoxazinyl.

The term “heterocyclylalkyl” refers to alkyl substituted with a heterocyclyl.

The term “heterocycloalkyl” refers to a fully saturated heterocyclyl.

A substituent is “substitutable” if it comprises at least one carbon, sulfur, oxygen or nitrogen atom that is bonded to one or more hydrogen atoms. Thus, for example, hydrogen, halogen, and cyano do not fall within this definition.

If a substituent is described as being “substituted,” a non-hydrogen substituent is in the place of a hydrogen substituent on a carbon or nitrogen of the substituent. Thus, for example, a substituted alkyl substituent is an alkyl substituent wherein at least one non-hydrogen substituent is in the place of a hydrogen substituent on the alkyl substituent. To illustrate, monofluoroalkyl is alkyl substituted with a fluoro substituent, and difluoroalkyl is alkyl substituted with two fluoro substituents. It should be recognized that if there are more than one substitutions on a substituent, each non-hydrogen substituent may be identical or different (unless otherwise stated).

If a substituent is described as being “optionally substituted,” the substituent may be either (1) not substituted, or (2) substituted. If a carbon of a substituent is described as being optionally substituted with one or more of a list of substituents, one or more of the hydrogens on the carbon (to the extent there are any) may separately and/or together be replaced with an independently selected optional substituent. If a nitrogen of a substituent is described as being optionally substituted with one or more of a list of substituents, one or more of the hydrogens on the nitrogen (to the extent there are any) may each be replaced with an independently selected optional substituent.

One exemplary substituent may be depicted as —NR′R,″ wherein R′ and R″ together with the nitrogen atom to which they are attached, may form a heterocyclic ring. The heterocyclic ring formed from R′ and R″ together with the nitrogen atom to which they are attached may be partially or fully saturated. In one embodiment, the heterocyclic ring consists of 3 to 7 atoms. In another embodiment, the heterocyclic ring is selected from the group consisting of pyrrolyl, imidazolyl, pyrazolyl, triazolyl, tetrazolyl, isoxazolyl, pyridyl and thiazolyl.

This specification uses the terms “substituent,” “radical,” and “group” interchangeably. If a group of substituents are collectively described as being optionally substituted by one or more of a list of substituents, the group may include: (1) unsubstitutable substituents, (2) substitutable substituents that are not substituted by the optional substituents, and/or (3) substitutable substituents that are substituted by one or more of the optional substituents. If a substituent is described as being optionally substituted with up to a particular number of non-hydrogen substituents, that substituent may be either (1) not substituted; or (2) substituted by up to that particular number of non-hydrogen substituents or by up to the maximum number of substitutable positions on the substituent, whichever is less. Thus, for example, if a substituent is described as a heteroaryl optionally substituted with up to 3 non-hydrogen substituents, then any heteroaryl with less than 3 substitutable positions would be optionally substituted by up to only as many non-hydrogen substituents as the heteroaryl has substitutable positions. To illustrate, tetrazolyl (which has only one substitutable position) would be optionally substituted with up to one non-hydrogen substituent. To illustrate further, if an amino nitrogen is described as being optionally substituted with up to 2 non-hydrogen substituents, then the nitrogen will be optionally substituted with up to 2 non-hydrogen substituents if the amino nitrogen is a primary nitrogen, whereas the amino nitrogen will be optionally substituted with up to only 1 non-hydrogen substituent if the amino nitrogen is a secondary nitrogen.

A prefix attached to a multi-moiety substituent only applies to the first moiety. To illustrate, the term “alkylcycloalkyl” contains two moieties: alkyl and cycloalkyl. Thus, the C₁-C₆-prefix on C₁-C₆-alkylcycloalkyl means that the alkyl moiety of the alkylcycloalkyl contains from 1 to 6 carbon atoms; the C₁-C₆-prefix does not describe the cycloalkyl moiety. To illustrate further, the prefix “halo” on haloalkoxyalkyl indicates that only the alkoxy moiety of the alkoxyalkyl substituent is substituted with one or more halogen substituents. If halogen substitution may alternatively or additionally occur on the alkyl moiety, the substituent would instead be described as “halogen-substituted alkoxyalkyl” rather than “haloalkoxyalkyl.” And finally, if the halogen substitution may only occur on the alkyl moiety, the substituent would instead be described as “alkoxyhaloalkyl.”

When a substituent is comprised of multiple moieties, unless otherwise indicated, it is the intention for the final moiety to serve as the point of attachment to the remainder of the molecule. For example, in a substituent A-B—C, moiety C is attached to the remainder of the molecule. In a substituent A-B—C-D, moiety D is attached to the remainder of the molecule. Similarly, in a substituent aminocarbonylmethyl, the methyl moiety is attached to the remainder of the molecule, where the substituent may also be be depicted as

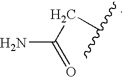

In a substituent trifluoromethylaminocarbonyl, the carbonyl moiety is attached to the remainder of the molecule, where the substituent may also be depicted as

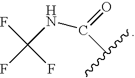

If substituents are described as being “independently selected” from a group, each substituent is selected independent of the other. Each substituent therefore may be identical to or different from the other substituent(s).

### B. Compounds

The present invention comprises, in part, a novel class of thieno[2,3-o]pyrimidine compounds. These compounds are useful as inhibitors of platelet mediated aggregation.

The present invention is directed, in part, to a class of compounds and pharmaceutically acceptable salts of the compounds or tautomers are disclosed, wherein the compounds have the structure of Formula I:

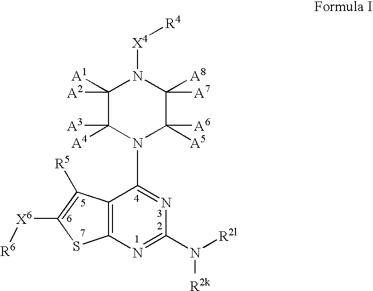

wherein:
A¹, A², A³, A⁴, A⁵, A⁶, A⁷ and A⁸ are independently selected from the group consisting of hydrogen, alkyl, and haloalkyl;
R^{2k} is selected from the group consisting of hydrogen, alkyl, alkenyl, alkynyl, cycloalkyl, aryl and heterocyclyl;
R^{2l} is selected from the group consisting of alkyl, alkenyl, alkynyl, cycloalkyl, aryl and heterocyclyl; wherein:
(a) the R^{2k} and R^{2l} alkenyl, alkynyl, cycloalkyl, aryl and heterocyclyl substituents may be optionally substituted with one or more substituents independently selected from the group consisting of halogen and —R^{2m};
(b) when the R^{2k} substituent is hydrogen and the R^{2l} substituent is alkyl, the R^{2l} alkyl substituent is substituted with one or more one substituents independently selected from the group consisting of chloro, bromo, iodo and —R^{2m};
(c) when both the R^{2k} substituent and R^{2l} substituent are alkyl, at least one of the R^{2k} and R^{2l} alkyl substituents is substituted with one or more one substituents independently selected from the group consisting of chloro, bromo, iodo and —R^{2m};
(d) when the R^{2k} substituent is other than hydrogen or alkyl and the R^{2l} substituent is alkyl, the R^{2l} alkyl substituent may be optionally substituted with one or more substituents independently selected from the group consisting of halogen and —R^{2m};
R^{2m} is selected from the group consisting of oxo, cyano, nitro, amino, alkyl, alkenyl, alkynyl, cycloalkyl, aryl, heterocyclyl, —C(O)R²ⁿ, —C(S)R²ⁿ, —C(O)OR²ⁿ, —C(S)OR²ⁿ, —C(O)SR²ⁿ, —C(O)NR²ⁿR^{2o},
—C(S)NR²ⁿR^{2o}, —OR²ⁿ, —OC(O)R²ⁿ, —OC(S)R²ⁿ, —NR²ⁿR^{2o}, —NR²ⁿC(O)R^{2o}, —NR²ⁿC(S)R^{2o}, —NR²ⁿC(O)OR^{2o},
—NR²ⁿC(S)OR^{2o}, —NR²ⁿS(O)₂R^{2o}, —NR²ⁿC(O)NR^{2o}R^{2p}, —S(O)_{q}R²ⁿ, —S(O)₂NR²ⁿR^{2o} and —SC(O)R²ⁿ;
q is 0, 1 or 2;
R²ⁿ, R^{2o} and R^{2p} are independently selected from the group consisting of hydrogen, alkyl, alkenyl, alkynyl, cycloalkyl, aryl and heterocyclyl;
wherein the R^{2m}, R²ⁿ, R^{2o} and R^{2p} alkyl, alkenyl, alkynyl, cycloalkyl, aryl, heterocyclyl substituents may be optionally substituted with one or more substituents independently selected from the group consisting of halogen, cyano, nitro, oxo, ═S, —R^{2q}, —C(O)R^{2q}, —C(S)R^{2q}, —C(O)OR^{2q}, C(S)OR^{2q}, —C(O)SR^{2q}, —C(O)NR^{2q}R^{2r}, —C(S)NR^{2q}R^{2r}, —OR^{2q}, —OC(O)R^{2r}, —OC(S)R^{2q}, —NR^{2q}R^{2r}, —NR^{2q}C(O)R^{2r}, —NR^{2q}C(S)R^{2r},
—NR^{2q}(O)OR^{2r}, NR^{2q}C(S)OR^{2r}, —NR^{2q}S(O)₂R^{2r}, —NR^{2q}C(O)NR^{2r}R^{2s}, —S(O)ᵣR^{2q}, —S(O)₂NR^{2q}R^{2r} and
—SC(O)R^{2q};
r is 0, 1 or 2;
R^{2q}, R^{2r} and R^{2s} are independently selected from the group consisting of hydrogen, alkyl, alkenyl, alkynyl, cycloalkyl, aryl and heterocyclyl;
wherein the R^{2q}, R^{2r} and R^{2s} alkyl, alkenyl, alkynyl, cycloalkyl, aryl and heterocyclyl substituents may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl;
X⁴ is selected from the group consisting of —C(O)—, —C(S)—, —S(O)— and —S(O)₂—;
R⁴ is selected from the group consisting of —R^{4l}, —OR^{4j}, and —NR^{4j}R^{4k};
wherein R^{4j} and R^{4k} are independently selected from the group consisting of hydrogen, alkyl, alkenyl, alkynyl, cycloalkyl, aryl, heterocyclyl, cycloalkylalkyl, arylalkyl, heterocyclylalkyl, arylcycloalkyl, heterocyclylcycloalkyl, cycloalkylaryl, cycloalkylheterocyclyl, arylaryl, heterocyclylheterocyclyl, arylheterocyclyl, heterocyclylaryl, cycloalkoxyalkyl, heterocyclyloxyalkyl, aryloxyaryl, heterocyclyloxyheterocyclyl, aryloxyheterocyclyl, heterocyclyloxyaryl, arylcarbonylaryl, heterocyclylcarbonylheterocyclyl, aryloxyalkyl, arylcarbonylheterocyclyl, heterocyclylcarbonylaryl, arylcarbonylaminoalkyl, heterocyclylcarbonylaminoalkyl, arylcarbonylaminoalkyl, and heterocyclylcarbonylaminoalkyl;
wherein the R^{4j} and R^{4k} substituents may be optionally substituted with one or more substituents independently selected from the group consisting of halogen, haloalkyl, hydroxyalkyl, oxo, ═S, nitro, cyano, —R^{4l}, —OR^{4l}, —C(O)R^{4l}, —C(O)OR^{4l}, C(O)NR⁴¹R^{4m}, —OC(O)R^{4l}, —ONR^{4l}R^{4m}, —NR^{4l}R^{4m},
—NR^{4l}C(O)R^{4m}, —NR^{4l}S(O)₂R^{4m}, —S(O)_{b}R^{4l}, —SC(O)R^{4l} and —SC(O)NR^{4l}R^{4m};
b is0, 1 or 2;
R^{4l} and R^{4m} are independently selected from the group consisting of hydrogen, alkyl, haloalkyl, alkenyl, cycloalkyl, aryl and heterocyclyl;
wherein the R^{4l} and R^{4m} alkyl, haloalkyl, alkenyl, cycloalkyl, aryl and heterocyclyl substituents may be optionally substituted with one or more substituents independently selected from the group consisting of halogen, hydroxy, cyano, oxo, ═S, nitro, —SH, amino, alkyl, haloalkyl, hydroxyalkyl, carboxy, alkoxy, alkoxycarbonyl and alkylamino;
R⁵ is selected from the group consisting of hydrogen, halogen, alkyl, haloalkyl, alkoxy and haloalkoxy;
X⁶ represents a bond or is —C(O)—; wherein:
(a) when X⁶ is —C(O)—, R⁶ is selected from the group consisting of —R^{6a} and —OR^{6a};
(b) when X⁶ represents a bond, R⁶ is selected from the group consisting of halogen, cyano, —R^{6a}

### and —OR^{6a};

R^{6a} is selected from the group consisting of hydrogen, alkyl, cycloalkyl and aryl; and

wherein the R^{6a} alkyl, cycloalkyl and aryl substituent may be optionally substituted with one or more substituents independently selected from the group consisting of halogen, hydroxy, oxo, ═S, cyano, alkyl, haloalkyl, hydroxyalkyl, cycloalkyl, carboxy,aryl and heterocyclyl.

In one embodiment of the compounds of Formula (I), A¹, A², A³, A⁴, A⁵, A⁶, A⁷ and A⁸ are each hydrogen. In another embodiment, A¹, A², A⁴, A⁵, A⁶, A⁷ and A⁸ are each hydrogen and A³ is methyl. In still another embodiment, A², A³, A⁴, A⁵, A⁶, A⁷ and A⁸ are each hydrogen and A¹ is methyl.

In another embodiment of the compounds of Formula (I), R⁵ is selected from the group consisting of hydrogen, halogen, alkyl, and —OR^{5a}, wherein the R⁵ alkyl substituent may be optionally substituted as provided in other embodiments herein, and R^{5a} is defined as provided in other embodiments herein. In another embodiment, R⁵ is selected from the group consisting of hydrogen, halogen, and alkyl, wherein the R⁵ alkyl substituent may be optionally substituted as above. In still another embodiment, R⁵ is selected from the group consisting of hydrogen, halogen and methyl. In still another embodiment, R⁵ is hydrogen.

In another embodiment of the compounds of Formula (I), R⁶ is selected from the group consisting of halogen, —R^{6a} and —OR^{6a}, wherein R^{6a} is defined as provided in other embodiments herein. In one embodiment, R⁶ is halogen. In another embodiment, R⁶ is fluorine. In another embodiment, R⁶ is chlorine. In another embodiment, R⁶ is bromine. In another embodiment, R⁶ is cyano.

In still another embodiment of Formula (I), X⁶ represents a bond and R⁶ is —R^{6a}, wherein R^{6a} is defined as provided in other embodiments herein. In still another embodiment, X⁶ is —C(O)— and R⁶ is —OR^{6a}, wherein R^{6a} is defined as provided in claim 1. In still another embodiment, R⁶is selected from the group consisting of —R^{6a} and —OR^{6a}, and R^{6a} is selected from the group consisting of hydrogen, alkyl, cycloalkyl, aryl and heterocyclyl, wherein the R^{6a} alkyl, cycloalkyl, aryl and heterocyclyl substituents may be optionally substituted as provided in other embodiments herein. In still another embodiment, R⁶ is selected from the group consisting of —R^{6a} and —OR^{6a}, and R^{6a} is selected from the group consisting of hydrogen, alkyl and aryl, wherein the R^{6a} alkyl and aryl substituents may be optionally substituted as provided in other embodiments herein. In still another embodiment, X⁶ represents a bond, R⁶ is —R^{6a}; and R^{6a} is hydrogen and alkyl, wherein the R^{6a} alkyl substituent may be optionally substituted as provided in other embodiments herein.

In still another embodiment of Formula (I), X⁶ represents a bond, R⁶ is —R^{6a}; and R^{6a} is hydrogen.

In still another embodiment of Formula (I), X⁶ represents a bond, R⁶ is —R^{6a}; and R^{6a} is selected from the group consisting of methyl, ethyl, propyl, butyl, pentyl, hexyl and phenyl. In still another embodiment, X⁶ represents a bond, R⁶ is —R^{6a}; and R^{6a} is selected from the group consisting of methyl, ethyl, propyl, butyl, pentyl, and hexyl. In still another embodiment, X⁶ represents a bond, R⁶ is —R^{6a}; and R^{6a} is selected from the group consisting of methyl, ethyl, propyl, butyl, and pentyl. In another embodiment, X⁶ represents a bond, R⁶ is —R^{6a}; and R^{6a} is unsubstituted alkyl.

In still another embodiment of Formula (I), X⁶ represents a bond, R⁶ is —R^{6a}; and R^{6a} is selected from the group consisting of methyl, ethyl, propyl, butyl, pentyl and hexyl, wherein said R^{6a} substituent is substituted with one or more halogen substituents. In still another embodiment, X⁶ represents a bond, R⁶ is —R^{6a}; and R^{6a} is selected from the group consisting of methyl, ethyl, propyl, butyl, pentyl and hexyl, wherein said R^{6a} substituent is substituted with one or more fluorine substituents. In another embodiment, X⁶ represents a bond, R⁶ is —R^{6a}; and R^{6a} is selected from the group consisting of methyl, ethyl, propyl, butyl, pentyl and hexyl, wherein said R^{6a} substituent is substituted with one or more chlorine substituents. In another embodiment, X⁶ represents a bond, R⁶ is —R^{6a}; and R^{6a} is selected from the group consisting of methyl, ethyl, propyl, butyl, pentyl and hexyl, wherein said R^{6a} substituent is substituted with one or more bromine substituents.

In another embodiment of the compounds of Formula (I), X⁴ is —C(O)— or —S(O)₂—. In still another embodiment of Formula (I), X⁴ is —C(O)—.

In another embodiment of the compounds of Formula (I), R⁴ is selected from the group consisting of —R^{4j}, —OR^{4j}, and —NR^{4j}R^{4k}; and R^{4j} and R^{4k} are independently selected from the group consisting of hydrogen, alkyl, alkenyl, alkynyl, cycloalkyl, aryl, and heterocyclyl, wherein the R^{4j}, and R^{4k} alkyl, alkenyl, alkynyl, cycloalkyl, aryl, and heterocyclyl substituents may be optionally substituted as provided in other embodiments herein. In another embodiment, R⁴ is selected from the group consisting of —R^{4j}, —OR^{4j}, and —NR^{4j}R^{4k}; R^{4j} is selected from the group consisting of hydrogen, alkyl, cycloalkyl, aryl, and heterocyclyl, wherein the R^{4j} alkyl, cycloalkyl, aryl, and heterocyclyl substituents may be optionally substituted as provided in other embodiments herein; and R^{4k} is selected from the group consisting of hydrogen and alkyl, wherein the R^{4k} alkyl substituent may be optionally substituted as provided in other embodiments herein.

In another embodiment of the compounds of Formula (I), R⁴ is selected from the group consisting of —R^{4j}, —OR^{4j}, and —NR^{4j}R^{4k}; and R^{4j} and R^{4k} are independently selected from the group consisting of hydrogen, alkyl, alkenyl, alkynyl, cycloalkyl, aryl, heterocyclyl, cycloalkylalkyl, arylalkyl, heterocyclylalkyl, arylcycloalkyl, heterocyclylcycloalkyl, cycloalkylaryl, cycloalkylheterocyclyl, arylaryl, heterocyclylheterocyclyl, arylheterocyclyl, heterocyclylaryl, cycloalkoxyalkyl, heterocyclyloxyalkyl, aryloxyaryl, heterocyclyloxyheterocyclyl, aryloxyheterocyclyl, heterocyclyloxyaryl, arylcarbonylaryl, heterocyclylcarbonylheterocyclyl, aryloxyalkyl, arylcarbonylheterocyclyl, heterocyclylcarbonylaryl, arylcarbonylaminoalkyl, heterocyclylcarbonylaminoalkyl, arylcarbonylaminoalkyl, and heterocyclylcarbonylaminoalkyl; wherein the R^{4j} and R^{4k} alkyl, alkenyl, alkynyl, cycloalkyl, aryl, heterocyclyl, cycloalkylalkyl, arylalkyl, heterocyclylalkyl, arylcycloalkyl, heterocyclylcycloalkyl, cycloalkylaryl, cycloalkylheterocyclyl, arylaryl, heterocyclylheterocyclyl, arylheterocyclyl, heterocyclylaryl, cycloalkoxyalkyl, heterocyclyloxyalkyl, aryloxyaryl, heterocyclyloxyheterocyclyl, aryloxyheterocyclyl, heterocyclyloxyaryl, arylcarbonylaryl, heterocyclylcarbonylheterocyclyl, aryloxyalkyl, arylcarbonylheterocyclyl, heterocyclylcarbonylaryl, arylcarbonylaminoalkyl, heterocyclylcarbonylaminoalkyl, arylcarbonylaminoalkyl, and heterocyclylcarbonylaminoalkyl substituents may be optionally substituted as provided in other embodiments herein.

In another embodiment of the compounds of Formula (I), R⁴ is selected from the group consisting of —R^{4j}, —OR^{4j}, and —NR^{4j}R^{4k}; and R^{4j} and R^{4k} are independently selected from the group consisting of hydrogen, alkyl, alkenyl, alkynyl, cycloalkyl, aryl, heterocyclyl, cycloalkylalkyl, arylalkyl, heterocyclylalkyl, arylcycloalkyl, heterocyclylcycloalkyl, cycloalkylaryl, cycloalkylheterocyclyl, arylaryl, heterocyclylheterocyclyl, arylheterocyclyl, heterocyclylaryl, cycloalkoxyalkyl, heterocyclyloxyalkyl, aryloxyaryl, heterocyclyloxyheterocyclyl, aryloxyheterocyclyl, heterocyclyloxyaryl, arylcarbonylaryl, heterocyclylcarbonylheterocyclyl, aryloxyalkyl, arylcarbonylheterocyclyl, heterocyclylcarbonylaryl, arylcarbonylaminoalkyl, heterocyclylcarbonylaminoalkyl, arylcarbonylaminoalkyl, and heterocyclylcarbonylaminoalkyl; wherein the R^{4j} and R^{4k} alkyl, alkenyl, alkynyl, cycloalkyl, aryl, heterocyclyl, cycloalkylalkyl, arylalkyl, heterocyclylalkyl, arylcycloalkyl, heterocyclylcycloalkyl, cycloalkylaryl, cycloalkylheterocyclyl, arylaryl, heterocyclylheterocyclyl, arylheterocyclyl, heterocyclylaryl, cycloalkoxyalkyl, heterocyclyloxyalkyl, aryloxyaryl, heterocyclyloxyheterocyclyl, aryloxyheterocyclyl, heterocyclyloxyaryl, arylcarbonylaryl, heterocyclylcarbonylheterocyclyl, aryloxyalkyl, arylcarbonylheterocyclyl, heterocyclylcarbonylaryl, arylcarbonylaminoalkyl, heterocyclylcarbonylaminoalkyl, arylcarbonylaminoalkyl, and heterocyclylcarbonylaminoalkyl substituents may be optionally substituted with one or more substituents independently selected from the group consisting of halogen, haloalkyl, hydroxyalkyl, oxo, ═S, nitro, cyano, —R^{4l}, —OR^{4l}, —C(O)R^{4l}, —C(O)OR^{4l}, —C(O)NR^{4l}R^{4m}, —OC(O)R^{4l}, —ONR^{4l}R^{4m}, —NR^{4l}R^{4m}, —NR^{4l}C(O)^{4m}, —NR^{4l}S(O)₂R^{4m}, —SR^{4l}, —S(O)R^{4l}, —S(O)₂R^{4l}, —SC(O)R^{4l} and —SC(O)NR^{4l}R^{4m}; wherein R^{4l} and R^{4m} are independently selected from the group consisting of hydrogen, alkyl, haloalkyl, alkenyl, cycloalkyl, aryl and heterocyclyl; and wherein the R^{4l} and R^{4m} alkyl, haloalkyl, alkenyl, cycloalkyl, aryl and heterocyclyl substituents may be optionally substituted as provided in other embodiments herein.

In another embodiment of the compounds of Formula (I), R^{2k} is selected from the group consisting of hydrogen and alkyl; and R^{2l} is selected from the group consisting of alkyl, alkenyl, alkynyl, cycloalkyl, aryl and heterocyclyl; wherein: (a) the R^{2l} alkenyl, alkynyl, cycloalkyl, aryl and heterocyclyl substituents may be optionally substituted as provided in other embodiments herein; (b) when the R^{2k} substituent is hydrogen and the R^{2l} substituent is alkyl, the R^{2l} alkyl substituent is substituted as provided in other embodiments herein; and (c) when both the R^{2k} substituent and R^{2l} substituent are alkyl, at least one of the R^{2k} and R^{2l} alkyl substituents as provided in other embodiments herein.

Another class of compounds of specific interest includes compounds, and pharmaceutically acceptable salts of the compounds, wherein the compounds have the structure of Formula II:

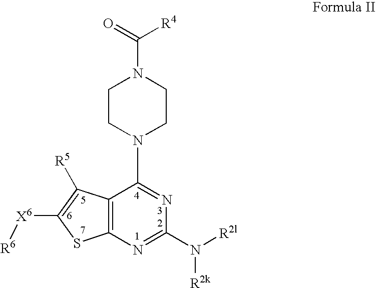

wherein R^{2k} is selected from the group consisting of hydrogen, alkyl, alkenyl, alkynyl, cycloalkyl, aryl and heterocyclyl;
R^{2l} is selected from the group consisting of alkyl, alkenyl, alkynyl, cycloalkyl, aryl and heterocyclyl; wherein:
(a) the R^{2k} and R^{2l} alkenyl, alkynyl, cycloalkyl, aryl and heterocyclyl substituents may be optionally substituted with one or more substituents independently selected from the group consisting of halogen and —R^{2m};
(b) when the R^{2k} substituent is hydrogen and the R^{2l} substituent is alkyl, the R^{2l} alkyl substituent is substituted with one or more one substituents independently selected from the group consisting of chloro, bromo, iodo and —R^{2m};
(c) when both the R^{2k} substituent and R^{2l} substituent are alkyl, at least one of the R^{2k} and R^{2l} alkyl substituents is substituted with one or more one substituents independently selected from the group consisting of chloro, bromo, iodo and —R^{2m};
(d) when the R^{2k} substituent is other than hydrogen or alkyl and the R^{2l} substituent is alkyl, the R^{2l} alkyl substituent may be optionally substituted with one or more substituents independently selected from the group consisting of halogen and —R^{2m};
R^{2m} is selected from the group consisting of cyano, nitro, amino, alkyl, alkenyl, alkynyl, cycloalkyl, aryl, heterocyclyl, —C(O)R²ⁿ, —C(S)R²ⁿ, —C(O)OR²ⁿ, —C(S)OR²ⁿ, —C(O)SR²ⁿ, —C(O)NR²ⁿR^{2o}, —C(S)NR²ⁿR^{2o}, —OR²ⁿ, —OC(O)R²ⁿ, —OC(S)R²ⁿ, —NR²ⁿR^{2o}, —NR²ⁿC(O)R^{2o}, —NR²ⁿC(S)R^{2o}, —NR²ⁿC(O)OR^{2o}, —NR²ⁿC(S)OR^{2o}, —NR²ⁿS(O)₂R^{2o}, —NR²ⁿC(O)NR^{2o}R^{2p}, —S(O)_{q}R²ⁿ, —S(O)₂NR²ⁿR^{2o} and —SC(O)R²ⁿ;
q is 0, 1 or 2;
R²ⁿ, R^{2o} and R^{2p} are independently selected from the group consisting of hydrogen, alkyl, alkenyl, alkynyl, cycloalkyl, aryl and heterocyclyl;
wherein the R^{2m}, R²ⁿ, R^{2o} and R^{2p} alkyl, alkenyl, alkynyl, cycloalkyl, aryl, heterocyclyl substituents may be optionally substituted with one or more substituents independently selected from the group consisting of halogen, cyano, nitro, oxo, ═S, —R^{2q}, —C(O)R^{2q}, —C(S)R^{2q}, —C(O)OR^{2q}, —C(S)OR^{2q}, —C(O)SR^{2q}, —C(O)NR^{2q}R^{2r}, —C(S)NR^{2q}R^{2r}, —OR^{2q}, —OC(O)R^{2r}, —OC(S)^{2q}, —N^{2q}R^{2r}, —NR^{2q}C(O)R^{2r}, —NR^{2q}C(S)R^{2r},
—NR^{2q}C(O)OR^{2r}, —NR^{2q}C(S)OR^{2r}, —NR^{2q}S(O)₂R^{2r}, —NR^{2q}C(O)NR^{2r}R^{2s}, —S(O)ᵣR^{2q}, —S(O)₂NR^{2q}R^{2r} and
—SC(O)R^{2q};
r is 0, 1 or 2;
R^{2q}, R^{2r} and R^{2s} are independently selected from the group consisting of hydrogen, alkyl, alkenyl, alkynyl, cycloalkyl, aryl and heterocyclyl;
wherein the R^{2q}, R^{2r} and R^{2s} alkyl, alkenyl, alkynyl, cycloalkyl, aryl and heterocyclyl substituents may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl;
R⁴ is selected from the group consisting of —R^{4j}, —OR^{4j}, and —NR^{4j}R^{4k};
wherein R^{4j} and R^{4k} are independently selected from the group consisting of hydrogen, alkyl, alkenyl, alkynyl, cycloalkyl, aryl, heterocyclyl, cycloalkylalkyl, arylalkyl, heterocyclylalkyl, arylcycloalkyl, heterocyclylcycloalkyl, cycloalkylaryl, cycloalkylheterocyclyl, arylaryl, heterocyclylheterocyclyl, arylheterocyclyl, heterocyclylaryl, cycloalkoxyalkyl, heterocyclyloxyalkyl, aryloxyaryl, heterocyclyloxyheterocyclyl, aryloxyheterocyclyl, heterocyclyloxyaryl, arylcarbonylaryl, heterocyclylcarbonylheterocyclyl, aryloxyalkyl, arylcarbonylheterocyclyl, heterocyclylcarbonylaryl, arylcarbonylaminoalkyl, heterocyclylcarbonylaminoalkyl, arylcarbonylaminoalkyl, and heterocyclylcarbonylaminoalkyl;
wherein the R^{4j} and R^{4k} substituents may be optionally substituted with one or more substituents independently selected from the group consisting of halogen, haloalkyl, hydroxyalkyl, oxo, ═S, nitro, cyano, —R^{4j}, —OR^{4l}, —C(O)R^{4l}, —C(O)OR^{4l}, —C(O)NR^{4l}R^{4m}, —OC(O)R^{4l}, —ONR^{4l}R^{4m}, —NR^{4l}R^{4m},
—NR^{4l}C(O)R^{4m}, —NR^{4l}S(O)₂R^{4m}, —S(O)_{b}R^{4l}, —SC(O)R^{4l} and —SC(O)NR^{4l}R^{4m};
b is 0, 1 or 2;
R^{4l} and R^{4m} are independently selected from the group consisting of hydrogen, alkyl, haloalkyl, alkenyl, cycloalkyl, aryl and heterocyclyl;
R⁵ is selected from the group consisting of hydrogen, halogen, alkyl, haloalkyl, alkoxy and haloalkoxy;
X⁶ represents a bond or is —C(O)—; wherein:
(a) when X⁶ is —C(O)—, R⁶ is selected from the group consisting of —R^{6a} and —OR^{6a};
(b) when X⁶ represents a bond, R⁶ is selected from the group consisting of halogen, cyano, —R^{6a}

### and —OR^{6a};

R^{6a} is selected from the group consisting of hydrogen, alkyl, cycloalkyl and aryl; and

wherein the R^{6a} alkyl, cycloalkyl and aryl substituent may be optionally substituted with one or more substituents independently selected from the group consisting of halogen, oxo, ═S, cyano, alkyl, haloalkyl, hydroxyalkyl, cycloalkyl, carboxy,aryl and heterocyclyl.

In another embodiment of the compounds of Formula (II), R⁵ is selected from the group consisting of hydrogen, halogen, alkyl, and —OR^{5a}, wherein the R⁵ alkyl substituent may be optionally substituted as provided in other embodiments herein, and R^{5a} is defined as provided in other embodiments herein. In another embodiment, R⁵ is selected from the group consisting of hydrogen, halogen, and alkyl, wherein the R⁵ alkyl substituent may be optionally substituted as above. In still another embodiment, R⁵ is selected from the group consisting of hydrogen, halogen and methyl. In still another embodiment, R⁵ is hydrogen.

In another embodiment of the compounds of Formula (II), R⁶ is selected from the group consisting of halogen, —R^{6a} and —OR^{6a}, wherein R^{6a} is defined as provided in other embodiments herein. In one embodiment, R⁶ is halogen. In another embodiment, R⁶ is fluorine. In another embodiment, R⁶ is chlorine. In another embodiment, R⁶ is bromine.

In still another embodiment of Formula (II), X⁶ represents a bond and R⁶ is —R^{6a}, wherein R^{6a} is defined as provided in other embodiments herein. In still another embodiment, X⁶ is —C(O)— and R⁶ is —OR^{6a}, wherein R^{6a} is defined as provided in claim 1. In still another embodiment, R⁶ is selected from the group consisting of —R^{6a} and —OR^{6a}, and R^{6a} is selected from the group consisting of hydrogen, alkyl, cycloalkyl, aryl and heterocyclyl, wherein the R^{6a} alkyl, cycloalkyl, aryl and heterocyclyl substituents may be optionally substituted as provided in other embodiments herein. In still another embodiment, R⁶ is selected from the group consisting of —R^{6a} and —OR^{6a}, and R^{6a} is selected from the group consisting of hydrogen, alkyl and aryl, wherein the R^{6a} alkyl and aryl substituents may be optionally substituted as provided in other embodiments herein. In still another embodiment, X⁶ represents a bond, R⁶ is —R^{6a}; and R^{6a} is hydrogen and alkyl, wherein the R^{6a} alkyl substituent may be optionally substituted as provided in other embodiments herein.

In still another embodiment of Formula (II), X⁶ represents a bond, R⁶ is —R^{6a}; and R^{6a} is hydrogen.

In still another embodiment of Formula (II), X⁶ represents a bond, R⁶ is —R^{6a}; and —R^{6a} is selected from the group consisting of methyl, ethyl, propyl, butyl, pentyl, hexyl and phenyl. In still another embodiment, X⁶ represents a bond, R⁶ is —R^{6a}; and R^{6a} is selected from the group consisting of methyl, ethyl, propyl, butyl, pentyl, and hexyl. In still another embodiment, X⁶ represents a bond, R⁶ is —R^{6a}; and R^{6a} is selected from the group consisting of methyl, ethyl, propyl, butyl, and pentyl. In another embodiment, X⁶ represents a bond, —R⁶ is —R^{6a}; and R^{6a} is unsubstituted alkyl.

In still another embodiment of Formula (II), X⁶ represents a bond, R⁶ is —R^{6a}; and R^{6a} is selected from the group consisting of methyl, ethyl, propyl, butyl, pentyl and hexyl, wherein said R^{6a} substituent is substituted with one or more halogen substituents. In still another embodiment, X⁶ represents a bond, R⁶ is —R^{6a}; and R^{6a} is selected from the group consisting of methyl, ethyl, propyl, butyl, pentyl and hexyl, wherein said R^{6a} substituent is substituted with one or more fluorine substituents. In another embodiment, X⁶ represents a bond, R⁶ is —R^{6a}; and R^{6a} is selected from the group consisting of methyl, ethyl, propyl, butyl, pentyl and hexyl, wherein said R^{6a} substituent is substituted with one or more chlorine substituents. In another embodiment, X⁶ represents a bond, R⁶ is —R^{6a}; and R^{6a} is selected from the group consisting of methyl, ethyl, propyl, butyl, pentyl and hexyl, wherein said R^{6a} substituent is substituted with one or more bromine substituents.

In another embodiment of the compounds of Formula (II), R⁴ is selected from the group consisting of —R^{4j}, —OR^{4j}, and —NR^{4j}R^{4k}; and R^{4j} and R^{4k} are independently selected from the group consisting of hydrogen, alkyl, alkenyl, alkynyl, cycloalkyl, aryl, and heterocyclyl, wherein the R^{4j} and R^{4k} alkyl, alkenyl, alkynyl, cycloalkyl, aryl, and heterocyclyl substituents may be optionally substituted as provided in other embodiments herein. In another embodiment, R⁴ is selected from the group consisting of —R^{4j}, —OR^{4j}, and —NR^{4j}R^{4k}; R^{4j} is selected from the group consisting of hydrogen, alkyl, cycloalkyl, aryl, and heterocyclyl, wherein the R^{4j} alkyl, cycloalkyl, aryl, and heterocyclyl substituents may be optionally substituted as provided in other embodiments herein; and R^{4k} is selected from the group consisting of hydrogen and alkyl, wherein the R^{4k} alkyl substituent may be optionally substituted as provided in other embodiments herein.

In another embodiment of the compounds of Formula (II), R⁴ is selected from the group consisting of —R^{4j}, —OR^{4j}, and —NR^{4j}R^{4k}; and R^{4j} and R^{4k} are independently selected from the group consisting of hydrogen, alkyl, alkenyl, alkynyl, cycloalkyl, aryl, heterocyclyl, cycloalkylalkyl, arylalkyl, heterocyclylalkyl, arylcycloalkyl, heterocyclylcycloalkyl, cycloalkylaryl, cycloalkylheterocyclyl, arylaryl, heterocyclylheterocyclyl, arylheterocyclyl, heterocyclylaryl, cycloalkoxyalkyl, heterocyclyloxyalkyl, aryloxyaryl, heterocyclyloxyheterocyclyl, aryloxyheterocyclyl, heterocyclyloxyaryl, arylcarbonylaryl, heterocyclylcarbonylheterocyclyl, aryloxyalkyl, arylcarbonylheterocyclyl, heterocyclylcarbonylaryl, arylcarbonylaminoalkyl, heterocyclylcarbonylaminoalkyl, arylcarbonylaminoalkyl, and heterocyclylcarbonylaminoalkyl; wherein the R^{4j} and R^{4k} alkyl, alkenyl, alkynyl, cycloalkyl, aryl, heterocyclyl, cycloalkylalkyl, arylalkyl, heterocyclylalkyl, arylcycloalkyl, heterocyclylcycloalkyl, cycloalkylaryl, cycloalkylheterocyclyl, arylaryl, heterocyclylheterocyclyl, arylheterocyclyl, heterocyclylaryl, cycloalkoxyalkyl, heterocyclyloxyalkyl, aryloxyaryl, heterocyclyloxyheterocyclyl, aryloxyheterocyclyl, heterocyclyloxyaryl, arylcarbonylaryl, heterocyclylcarbonylheterocyclyl, aryloxyalkyl, arylcarbonylheterocyclyl, heterocyclylcarbonylaryl, arylcarbonylaminoalkyl, heterocyclylcarbonylaminoalkyl, arylcarbonylaminoalkyl, and heterocyclylcarbonylaminoalkyl substituents may be optionally substituted as provided in other embodiments herein.

In another embodiment of the compounds of Formula (II), R⁴ is selected from the group consisting of —R^{4j}, —OR^{4j}, and —NR^{4j}R^{4k}; and R^{4j} and R^{4k} are independently selected from the group consisting of hydrogen, alkyl, alkenyl, alkynyl, cycloalkyl, aryl, heterocyclyl, cycloalkylalkyl, arylalkyl, heterocyclylalkyl, arylcycloalkyl, heterocyclylcycloalkyl, cycloalkylaryl, cycloalkylheterocyclyl, arylaryl, heterocyclylheterocyclyl, arylheterocyclyl, heterocyclylaryl, cycloalkoxyalkyl, heterocyclyloxyalkyl, aryloxyaryl, heterocyclyloxyheterocyclyl, aryloxyheterocyclyl, heterocyclyloxyaryl, arylcarbonylaryl, heterocyclylcarbonylheterocyclyl, aryloxyalkyl, arylcarbonylheterocyclyl, heterocyclylcarbonylaryl, arylcarbonylaminoalkyl, heterocyclylcarbonylaminoalkyl, arylcarbonylaminoalkyl, and heterocyclylcarbonylaminoalkyl; wherein the R^{4j} and R^{4k} alkyl, alkenyl, alkynyl, cycloalkyl, aryl, heterocyclyl, cycloalkylalkyl, arylalkyl, heterocyclylalkyl, arylcycloalkyl, heterocyclylcycloalkyl, cycloalkylaryl, cycloalkylheterocyclyl, arylaryl, heterocyclylheterocyclyl, arylheterocyclyl, heterocyclylaryl, cycloalkoxyalkyl, heterocyclyloxyalkyl, aryloxyaryl, heterocyclyloxyheterocyclyl, aryloxyheterocyclyl, heterocyclyloxyaryl, arylcarbonylaryl, heterocyclylcarbonylheterocyclyl, aryloxyalkyl, arylcarbonylheterocyclyl, heterocyclylcarbonylaryl, arylcarbonylaminoalkyl, heterocyclylcarbonylaminoalkyl, arylcarbonylaminoalkyl, and heterocyclylcarbonylaminoalkyl substituents may be optionally substituted with one or more substituents independently selected from the group consisting of halogen, haloalkyl, hydroxyalkyl, oxo, ═S, nitro, cyano, —R^{4l}, —OR^{4l}, —C(O)R^{4l}, —C(O)OR^{4l}, —C(O)NR^{4l}R^{4m}, —OC(O)R^{4l}, —ONR^{4l}R^{4m}, —NR^{4l}R^{4m}, —NR^{4l}C(O)R^{4m}, —NR^{4l}S(O)₂R^{4m}, —SR^{4l}, —S(O)R^{4l}, —S(O)₂R^{4l}, —SC(O)R^{4l} and —SC(O)NR^{4l}R^{4m}; wherein R^{4l} and R^{4m} are independently selected from the group consisting of hydrogen, alkyl, haloalkyl, alkenyl, cycloalkyl, aryl and heterocyclyl; and wherein the R^{4l} and R^{4m} alkyl, haloalkyl, alkenyl, cycloalkyl, aryl and heterocyclyl substituents may be optionally substituted as provided in other embodiments herein.

In another embodiment of the compounds of Formula (II), R^{2k} is selected from the group consisting of hydrogen and alkyl; and R^{2l} is selected from the group consisting of alkyl, alkenyl, alkynyl, cycloalkyl, aryl and heterocyclyl; wherein: (a) the R^{2l} alkenyl, alkynyl, cycloalkyl, aryl and heterocyclyl substituents may be optionally substituted as provided in other embodiments herein; (b) when the R^{2k} substituent is hydrogen and the R^{2l} substituent is alkyl, the R^{2l} alkyl substituent is substituted as provided in other embodiments herein; and (c) when both the R^{2k} substituent and R^{2l} substituent are alkyl, at least one of the R^{2k} and R^{2l} alkyl substituents as provided in other embodiments herein.

Another class of compounds of specific interest includes compounds, and pharmaceutically acceptable salts of the compounds, wherein the compounds have the structure of Formula III:

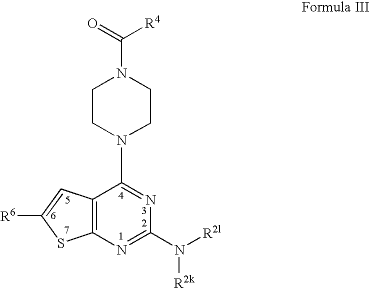

wherein R^{2k} is selected from the group consisting of hydrogen, alkyl, alkenyl, alkynyl, cycloalkyl, aryl and heterocyclyl;
R^{2l} is selected from the group consisting of alkyl, alkenyl, alkynyl, cycloalkyl, aryl and heterocyclyl; wherein:
(a) the R^{2k} and R^{2l} alkenyl, alkynyl, cycloalkyl, aryl and heterocyclyl substituents may be optionally substituted with one or more substituents independently selected from the group consisting of halogen and —R^{2m};
(b) when the R^{2k} substituent is hydrogen and the R^{2l} substituent is alkyl, the R^{2l} alkyl substituent is substituted with one or more one substituents independently selected from the group consisting of chloro, bromo, iodo and —R^{2m};
(c) when both the R^{2k} substituent and R^{2l} substituent are alkyl, at least one of the R^{2k} and R^{2l} alkyl substituents is substituted with one or more one substituents independently selected from the group consisting of chloro, bromo, iodo and —R^{2m};
(d) when the R^{2k} substituent is other than hydrogen or alkyl and the R^{2l} substituent is alkyl, the R^{2l} alkyl substituent may be optionally substituted with one or more substituents independently selected from the group consisting of halogen and —R^{2m};
R^{2m} is selected from the group consisting of cyano, nitro, amino, alkyl, alkenyl, alkynyl, cycloalkyl, aryl, heterocyclyl, —C(O)R²ⁿ, —C(S)R²ⁿ, —C(O)OR²ⁿ, —C(S)OR²ⁿ, —C(O)SR²ⁿ, —C(O)NR²ⁿR^{2o}, —C(S)NR²ⁿR^{2o},
13 OR²ⁿ, —OC(O)R²ⁿ, —OC(S)R²ⁿ, —NR²ⁿR^{2o}, —NR²ⁿC(O)R^{2o}, —NR²ⁿC(S)R^{2o}, —NR²ⁿC(O)OR^{2o}, —NR²ⁿC(S)OR^{2o}, —NR²ⁿS(O)₂R^{2o}, —NR²ⁿC(O)N R^{2o}R^{2p}, —S(O)_{q}R²ⁿ, —S(O)₂NR²ⁿR^{2o} and —SC(O)R²ⁿ;
q is 0, 1 or 2;
R²ⁿ, R^{2o} and R^{2p} are independently selected from the group consisting of hydrogen, alkyl, alkenyl, alkynyl, cycloalkyl, aryl and heterocyclyl;
wherein the R^{2m}, R²ⁿ, R^{2o} and R^{2p} alkyl, alkenyl, alkynyl, cycloalkyl, aryl, heterocyclyl substituents may be optionally substituted with one or more substituents independently selected from the group consisting of halogen, cyano, nitro, oxo, ═S, —R^{2q}, —C(O)R^{2q}, —C(S)R^{2q}, —C(O)OR^{2q}, —C(S)OR^{2q}, —C(O)SR^{2q}, —C(O)NR^{2q}R^{2r}, —C(S)NR^{2q}R^{2r}, —OR^{2q}, —OC(O)R^{2r}, —OC(S)R^{2q}, —NR^{2q}R^{2r}, —NR^{2q}C(O)R^{2r}, —NR^{2q}C(S)R^{2r},
—NR^{2q}(O)OR^{2r}, NR^{2q}C(S)OR^{2r}, —NR^{2q}S(O)₂R^{2r}, —NR_{2q}C(O)NR^{2r}R^{2s}, —S(O)ᵣR^{2q}, −S(O)₂NR^{2q}R^{2r} and
—SC(O)R^{2q};
r is 0, 1 or 2;
R^{2q}, R^{2r} and R^{2s} are independently selected from the group consisting of hydrogen, alkyl, alkenyl, alkynyl, cycloalkyl, aryl and heterocyclyl;
wherein the R^{2q}, R^{2r} and R^{2s} alkyl, alkenyl, alkynyl, cycloalkyl, aryl and heterocyclyl substituents may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl;
R⁴ is selected from the group consisting of —R^{4j}, —OR^{4j}, and —NR^{4j}R^{4k};
wherein R^{4j} and R^{4k} are independently selected from the group consisting of hydrogen, alkyl, alkenyl, alkynyl, cycloalkyl, aryl, heterocyclyl, cycloalkylalkyl, arylalkyl, heterocyclylalkyl, arylcycloalkyl, heterocyclylcycloalkyl, cycloalkylaryl, cycloalkylheterocyclyl, arylaryl, heterocyclylheterocyclyl, arylheterocyclyl, heterocyclylaryl, cycloalkoxyalkyl, heterocyclyloxyalkyl, aryloxyaryl, heterocyclyloxyheterocyclyl, aryloxyheterocyclyl, heterocyclyloxyaryl, arylcarbonylaryl, heterocyclylcarbonylheterocyclyl, aryloxyalkyl, arylcarbonylheterocyclyl, heterocyclylcarbonylaryl, arylcarbonylaminoalkyl, heterocyclylcarbonylaminoalkyl, arylcarbonylaminoalkyl, and heterocyclylcarbonylaminoalkyl;
wherein the R^{4j} and R^{4k} substituents may be optionally substituted with one or more substituents independently selected from the group consisting of halogen, haloalkyl, hydroxyalkyl, oxo, ═S, nitro, cyano, —R^{4l}, —OR^{4l}, —C(O)R^{4l}, —C(O)OR^{4l}, C(O)NR^{4l}R^{4m}, —OC(O)R^{4l}, —ONR^{4l}R^{4m}, —NR^{4l}R^{4m}, —NR^{4l}C(O)R^{4m},
—NR^{4l}S(O)₂R^{4m}, —S(O)_{b}R^{4l}, —SC(O)R^{4l} and —SC(O)NR^{4l}R^{4m};
b is 0, 1 or 2;
R^{4l} and R^{4m} are independently selected from the group consisting of hydrogen, alkyl, haloalkyl, alkenyl, cycloalkyl, aryl and heterocyclyl;
R⁶ is selected from the group consisting of halogen, cyano, —R^{6a} and —OR^{6a};
R^{6a} is selected from the group consisting of hydrogen, alkyl, cycloalkyl and aryl; and
wherein the R^{6a} alkyl, cycloalkyl and aryl substituent may be optionally substituted with one or more substituents independently selected from the group consisting of halogen, oxo, ═S, cyano, alkyl, haloalkyl, hydroxyalkyl, cycloalkyl, carboxy,aryl and heterocyclyl.

In another embodiment of the compounds of Formula (III), R⁶ is selected from the group consisting of —R^{6a} and —OR^{6a}, wherein R^{6a} is defined as provided in other embodiments herein. In still another embodiment of the compounds of Formula (III), R⁶ is selected from the group consisting of —R^{6a} and —OR^{6a}; and R^{6a} is selected from the group consisting of hydrogen, alkyl, cycloalkyl and aryl, wherein the R^{6a} alkyl, cycloalkyl and aryl substituents may be optionally substituted as provided in other embodiments herein.

In another embodiment of the compounds of Formula (III), R^{6a} is alkyl, wherein the R^{6a} alkyl substituent may be optionally substituted as provided in other embodiments herein. In still another embodiment of the compounds of Formula (III), R^{6a} is unsubstituted alkyl.

In another embodiment of the compounds of Formula (III), R⁴ is —NR^{4j}R^{4k}; wherein the R^{4j} and R^{4k} substituents may be optionally substituted as provided in other embodiments herein. In still another embodiment of the compounds of Formula (III), R^{4j} and R^{4k} are independently selected from the group consisting of hydrogen, alkyl, cycloalkyl and aryl, wherein the R^{4j} and R^{4k} alkyl and aryl may be optionally substituted as provided in other embodiments herein. In still another embodiment, R^{4j} and R^{4k} are independently selected from the group consisting of hydrogen, methyl, ethyl, propyl, butyl, phenyl, phenylphenyl, phenylmethyl, phenylethyl, phenylpropyl, and phenylbutyl; and wherein the R^{4j} and R^{4k} methyl, ethyl, propyl, butyl, phenyl, phenylphenyl, phenylmethyl, phenylethyl, phenylpropyl, and phenylbutyl may be optionally substituted as provided in other embodiments herein.

In another embodiment of the compounds of Formula (III), R⁴ is —NR^{4j}R^{4k}; wherein R^{4j} and R^{4k} are independently selected from the group consisting of hydrogen, phenylmethyl and phenylphenyl; and wherein the R^{4j} and R^{4k} phenylmethyl and phenylphenyl may be optionally substituted as provided in other embodiments herein.

In another embodiment of the compounds of Formula (III), R⁴ is —R^{4j} or —OR^{4j}; wherein R^{4j} is selected from the group consisting of alkyl, aryl, cycloalkyl, heterocyclyl, arylaryl, arylalkyl, heterocyclylalkyl, arylcycloalkyl, cycloalkylaryl, arylheterocyclyl, aryloxyaryl, heterocyclyloxyaryl, arylcarbonylaryl, and arylcarbonylaminoalkyl; and wherein the R^{4j} substituents may be optionally substituted as provided in other embodiments herein.

In another embodiment of the compounds of Formula (III), R⁴ is —R^{4j} or —OR^{4j}; wherein R^{4j} is selected from the group consisting of (C₁-C₆)-alkyl, (C₃-C₆)-cycloalkyl, (C₃-C₁₀)-aryl, (C₃-C₁₄)-heterocyclyl, (C₃-C₁₀)-aryl -(C₁-C₆)-alkyl, (C₃-C₁₄)-heterocyclyl-(C₁-C₆)-alkyl, (C₃-C₁₄)-cycloalkyl, (C₃-C₆)-cycloalkyl-(C₃-C₁₀)-aryl, (C₃-C₁₀)-aryl-(C₃-C₁₄)-heterocyclyl, (C₃-C₁₀)-aryl-O-(C₃-C₁₀)-aryl, (C₃-C₁₀)-aryl-(C₃-C₁₀)-aryl, (C₃-C₁₄)-heterocyclyl-O—(C₃-C₁₀)-aryl, (C₃-C₁₀)-aryl-C(O)—(C₃-C₁₀)-aryl, (C₃-C₁₀)-aryl-O—(C₁-C₆)-alkyl, and (C₃-C₁₀)-aryl-C(O)-amino-(C₁-C₆)-alkyl; wherein the R^{4j} substituents may be optionally substituted as provided in other embodiments herein.

In another embodiment of the compounds of Formula (III), R⁴ is —R^{4j} or —OR^{4j}; wherein R^{4j} is selected from the group consisting of methyl, ethyl, propyl, butyl, cyclopropyl, cyclobutyl, phenyl, naphthyl, anthracenyl, pyrrolidinyl, pyrrolinyl, pyrrolyl, tetrahydrofuranyl, furanyl, dioxolanyl, imidazolidinyl, imidazolynyl, imidazolyl, pyrazolidinyl, pyrazolinyl, pyrazolyl, oxazolyl, isoxazolyl, oxadiazolyl, thiophenyl, thiazolyl, thiadiazolyl, triazolyl, piperidinyl, pyridinyl, piperazinyl, pyrazinyl, pyrimidinyl, pyridazinyl, triazinyl, morpholinyl, dioxanyl, tetrahydro-2H-pyranyl, 2H-pyranyl, 4H-pyranyl, thiomorpholinyl, indolyl, dihydrobenzofuranyl, quinolinyl and fluorenyl; and wherein the R^{4j} substituents may be optionally substituted as provided in other embodiments herein.

In still another embodiment of the compounds of Formula (III), R⁴ is —R^{4j} or —OR^{4j}; wherein R^{4j} is selected from the group consisting of phenylphenyl, phenylnaphthyl, phenylanthracenyl, naphthylphenyl, naphthylnaphthyl, naphthylanthracenyl, anthracenylphenyl, anthracenyinaphthyl and anthracenylanthracenyl; and wherein the R^{4j} substituents may be optionally substituted as provided in other embodiments herein.

In still another embodiment of the compounds of Formula (III), R⁴ is —R^{4j} or —OR^{4j}; wherein R^{4j} is selected from the group consisting of phenylmethyl, phenylethyl, phenylpropyl, phenylbutyl, naphthylmethyl, naphthylethyl, naphthylpropyl, naphthylbutyl, anthracenylmethyl, anthracenylethyl, anthracenylpropyl, anthracenylbutyl, phenylcyclopropyl, phenylcyclobutyl, phenylcyclopentyl, phenylcyclohexyl, naphthylcyclopropyl, naphthylcyclobutyl, naphthylcyclopentyl, naphthylcyclohexyl, anthracenylcyclopropyl, anthracenylcyclobutyl, anthracenylcyclopentyl, anthracenylcyclohexyl, cyclopropylphenyl, cyclopropyinaphthyl, cyclopropylanthracenyl, cyclobutylphenyl, cyclobutyinaphthyl, cyclobutylanthracenyl, cyclopentylphenyl, cyclopentyinaphthyl, cyclopentylanthracenyl, cyclohexylphenyl, cyclohexylnaphthyl, cyclohexylanthracenyl, phenylphenylmethyl, phenylphenylethyl, phenylphenylpropyl, phenylphenylbutyl, diphenylmethyl, diphenylethyl, diphenylpropyl and diphenylbutyl; and wherein the R^{4j} substituents may be optionally substituted as provided in other embodiments herein.

In another embodiment of the compounds of Formula (III), R⁴ is —R^{4j} or —OR^{4j}; wherein R^{4j} is selected from the group consisting of phenyloxymethyl, phenyloxyethyl, phenyloxypropyl, phenyloxybutyl, naphthyloxymethyl, naphthyloxyethyl, naphthyloxypropyl, naphthyloxybutyl, anthracenyloxymethyl, anthracenyloxyethyl, anthracenyloxypropyl, anthracenyloxybutyl, methoxyphenyl, ethoxyphenyl, propoxyphenyl, butoxyphenyl, methoxynaphthyl, ethoxynaphthyl, propoxynaphthyl, butoxynaphthyl, phenyloxyphenyl, phenyloxynaphthyl, phenyloxyanthracenyl, naphthyloxyphenyl, naphthyloxynaphthyl, naphthyloxyanthracenyl, anthracenyloxyphenyl, anthracenyloxynaphthyl and anthracenyloxyanthracenyl; wherein the R^{4j} substituents may be optionally substituted as provided in other embodiments herein.

In another embodiment of the compounds of Formula (III), R⁴ is —R^{4j} or —OR^{4j}; wherein R^{4j} is selected from the group consisting of phenylcarbonylphenyl, phenylcarbonylnaphthyl, phenylcarbonylanthracenyl, naphthylcarbonylphenyl, naphthylcarbonylnaphthyl, naphthylcarbonylanthracenyl, anthracenylcarbonylphenyl, anthracenylcarbonylnaphthyl, anthracenylcarbonylanthracenyl, phenylcarbonylaminomethyl, phenylcarbonylaminoethyl, phenylcarbonylaminopropyl, phenylcarbonylaminobutyl, naphthylcarbonylaminomethyl, naphthylcarbonylaminoethyl, naphthylcarbonylaminopropyl, naphthylcarbonylaaminobutyl, anthracenylcarbonylaminomethyl, anthracenylcarbonylaminoethyl, anthracenylcarbonylaminopropyl, anthracenylcarbonylaminobutyl, phenylcarbonylaminomethyl, phenylcarbonylaminoethyl, phenylcarbonylaminopropyl and phenylcarbonylaminobutyl; and wherein the R^{4j} substituents may be optionally substituted as provided in other embodiments herein.

In another embodiment of the compounds of Formula (III), R⁴ is —R^{4j} or —OR^{4j}; wherein R^{4j} is selected from the group consisting of pyrrolidinylmethyl, pyrrolidinylethyl, pyrrolidinylpropyl, pyrrolidinylbutyl, pyrrolinylmethyl, pyrrolinylethyl, pyrrolinylpropyl, pyrrolinylbutyl, pyrrolylmethyl, pyrrolylethyl, pyrrolylpropyl, pyrrolylbutyl, tetrahydrofuranylmethyl, tetrahydrofuranylethyl, tetrahydrofuranylpropyl, tetrahydrofuranylbutyl, furanylmethyl, furanylethyl, furanylpropyl, furanylbutyl, dioxolanylmethyl, dioxolanylethyl, dioxolanylpropyl, dioxolanylbutyl, imidazolidinylmethyl, imidazolidinylethyl, imidazolidinylpropyl, imidazolidinylbutyl, imidazolynylmethyl, imidazolynylethyl, imidazolynylpropyl, imidazolynylbutyl, imidazolylmethyl, imidazolylethyl, imidazolylpropyl, imidazolylbutyl, pyrazolidinylmethyl, pyrazolidinylethyl, pyrazolidinylpropyl, pyrazolidinylbutyl, pyrazolinylmethyl, pyrazolinylethyl, pyrazolinylpropyl, pyrazolinylbutyl, pyrazolylmethyl, pyrazolylethyl, pyrazolylpropyl, pyrazolylbutyl, oxazolylmethyl, oxazolylethyl, oxazolylpropyl, oxazolylbutyl, isoxazolylmethyl, isoxazolylethyl, isoxazolylpropyl, isoxazolylbutyl, oxadiazolylmethyl, oxadiazolylethyl, oxadiazolylpropyl, oxadiazolylbutyl, thiophenylmethyl, thiophenylethyl, thiophenylpropyl, thiophenylbutyl, thiazolylmethyl, thiazolylethyl, thiazolylpropyl, thiazolylbutyl, thiadiazolylmethyl, thiadiazolylethyl, thiadiazolylpropyl, thiadiazolylbutyl, triazolylmethyl, triazolylethyl, triazolylpropyl, triazolylbutyl, piperidinylmethyl, piperidinylethyl, piperidinylpropyl, piperidinylbutyl, pyridinylmethyl, pyridinylethyl, pyridinylpropyl, pyridinylbutyl, piperazinylmethyl, piperazinylethyl, piperazinylpropyl, piperazinylbutyl, pyrazinylmethyl, pyrazinylethyl, pyrazinylpropyl, pyrazinylbutyl, pyrimidinylmethyl, pyrimidinylethyl, pyrimidinylpropyl, pyrimidinylbutyl, pyridazinylmethyl, pyridazinylethyl, pyridazinylpropyl, pyridazinylbutyl, triazinylmethyl, triazinylethyl, triazinylpropyl, triazinylbutyl, morpholinylmethyl, morpholinylethyl, morpholinylpropyl, morpholinylbutyl, dioxanylmethyl, dioxanylethyl, dioxanylpropyl, dioxanylbutyl, tetrahydro-2H-pyranylmethyl, tetrahydro-2H-pyranylethyl, tetrahydro-2H-pyranylpropyl, tetrahydro-2H-pyranylbutyl, 2H-pyranylmethyl, 2H-pyranylethyl, 2H-pyranylpropyl, 2H-pyranylbutyl, 4H-pyranylmethyl, 4H-pyranylethyl, 4H-pyranylpropyl, 4H-pyranylbutyl, thiomorpholinylmethyl, thiomorpholinylethyl, thiomorpholinylpropyl, thiomorpholinylbutyl, quinolinylmethyl, quinolinylethyl, quinolinylpropyl, quinolinylbutyl, fluorenylmethyl, fluorenylethyl, fluorenylpropyl and fluorenylbutyl; and wherein the R^{4j} substituents may be optionally substituted as provided in other embodiments herein.

In another embodiment of the compounds of Formula (III), R⁴ is —R^{4j} or —OR^{4j}; wherein R^{4j} is selected from the group consisting of phenylpyrrolidinyl, naphthylpyrrolidinyl, anthracenylpyrrolidinyl, phenylpyrrolinyl, naphthylpyrrolinyl, anthracenylpyrrolinyl, phenylpyrrolyl, naphthylpyrrolyl, anthracenylpyrrolyl, phenyltetrahydrofuranyl, naphthyltetrahydrofuranyl, anthracenyltetrahydrofuranyl, phenylfuranyl, naphthylfuranyl, anthracenylfuranyl, phenyldioxolanyl, naphthyldioxolanyl, anthracenyldioxolanyl, phenylimidazolidinyl, naphthylimidazolidinyl, anthracenylimidazolidinyl, phenylimidazolynyl, naphthylimidazolynyl, anthracenylimidazolynyl, phenylimidazolyl, naphthylimidazolyl, anthracenylimidazolyl, phenylpyrazolidinyl, naphthylpyrazolidinyl, anthracenylpyrazolidinyl, phenylpyrazolinyl, naphthylpyrazolinyl, anthracenylpyrazolinyl, phenylpyrazolyl, naphthylpyrazolyl, anthracenylpyrazolyl, phenyloxazolyl, naphthyloxazolyl, anthracenyloxazolyl, phenylisoxazolyl, naphthylisoxazolyl, anthracenylisoxazolyl, phenyl-oxadiazolyl, naphthyl-oxadiazolyl, anthracenyl-oxadiazolyl, phenylthiophenyl, naphthylthiophenyl, anthracenylthiophenyl, phenylthiazolyl, naphthylthiazolyl, anthracenylthiazolyl, phenylthiadiazolyl, naphthylthiadiazolyl, anthracenylthiadiazolyl, phenyltriazolyl, naphthyltriazolyl, anthracenyltriazolyl, phenylpiperidinyl, naphthylpiperidinyl, anthracenylpiperidinyl, phenylpyridinyl, naphthylpyridinyl, anthracenylpyridinyl, phenylpiperazinyl, naphthylpiperazinyl, anthracenylpiperazinyl, phenylpyrazinyl, naphthylpyrazinyl, anthracenyipyrazinyl, phenylpyrimidinyl, naphthylpyrimidinyl, anthracenylpyrimidinyl, phenylpyridazinyl, naphthylpyridazinyl, anthracenylpyridazinyl, phenyltriazinyl, naphthyltriazinyl, anthracenyltriazinyl, phenylmorpholinyl, naphthylmorpholinyl, anthracenylmorpholinyl, phenyldioxanyl, naphthyldioxanyl, anthracenyldioxanyl, phenyltetrahydro-2H-pyranyl, naphthyltetrahydro-2H-pyranyl, anthracenyltetrahydro-2H-pyranyl, phenyl-2H-pyranyl, naphthyl-2H-pyranyl, anthracenyl-2H-pyranyl, phenyl-4H-pyranyl, naphthyl-4H-pyranyl, anthracenyl-4H-pyranyl, phenylthiomorpholinyl, naphthylthiomorpholinyl, anthracenylthiomorpholinyl, phenylquinolinyl, naphthylquinolinyl, anthracenylquinolinyl, phenylfluorenyl, naphthylfluorenyl and anthracenylfluorenyl; and wherein the R^{4j} substituents may be optionally substituted as provided in other embodiments herein.

In another embodiment of the compounds of Formula (III), R⁴ is —R^{4j} or —OR^{4j}; wherein R^{4j} is selected from the group consisting of pyrrolidinyloxyphenyl, pyrrolidinyloxynaphthyl, pyrrolidinyloxyanthracenyl, pyrrolinyloxyphenyl, pyrrolinyloxynaphthyl, pyrrolinyloxyanthracenyl, pyrrolyloxyphenyl, pyrrolyloxynaphthyl, pyrrolyloxyanthracenyl, tetrahydrofuranyloxyphenyl, tetrahydrofuranyloxynaphthyl, tetrahydrofuranyloxyanthracenyl, furanyloxyphenyl, furanyloxynaphthyl, furanyloxyanthracenyl, dioxolanyloxyphenyl, dioxolanyloxynaphthyl, dioxolanyloxyanthracenyl, imidazolidinyloxyphenyl, imidazolidinyloxynaphthyl, imidazolidinyloxyanthracenyl, imidazolynyloxyphenyl, imidazolynyloxynaphthyl, imidazolynyloxyanthracenyl, imidazolyloxyphenyl, imidazolyloxynaphthyl, imidazolyloxyanthracenyl, pyrazolidinyloxyphenyl, pyrazolidinyloxynaphthyl, pyrazolidinyloxyanthracenyl, pyrazolinyloxyphenyl, pyrazolinyloxynaphthyl, pyrazolinyloxyanthracenyl, pyrazolyloxyphenyl, pyrazolyloxynaphthyl, pyrazolyloxyanthracenyl, oxazolyloxyphenyl, oxazolyloxynaphthyl, oxazolyloxyanthracenyl, isoxazolyloxyphenyl, isoxazolyloxynaphthyl, isoxazolyloxyanthracenyl, oxadiazolyloxyphenyl, oxadiazolyloxynaphthyl, oxadiazolyloxyanthracenyl, thiophenyloxyphenyl, thiophenyloxynaphthyl, thiophenyloxyanthracenyl, thiazolyloxyphenyl, thiazolyloxynaphthyl, thiazolyloxyanthracenyl, thiadiazolyloxyphenyl, thiadiazolyloxynaphthyl, thiadiazolyloxyanthracenyl, triazolyloxyphenyl, triazolyloxynaphthyl, triazolyloxyanthracenyl, piperidinyloxyphenyl, piperidinyloxynaphthyl, piperidinyloxyanthracenyl, pyridinyloxyphenyl, pyridinyloxynaphthyl, pyridinyloxyanthracenyl, piperazinyloxyphenyl, piperazinyloxynaphthyl, piperazinyloxyanthracenyl, pyrazinyloxyphenyl, pyrazinyloxynaphthyl, pyrazinyloxyanthracenyl, pyrimidinyloxyphenyl, pyrimidinyloxynaphthyl, pyrimidinyloxyanthracenyl, pyridazinyloxyphenyl, pyridazinyloxynaphthyl, pyridazinyloxyanthracenyl, triazinyloxyphenyl, triazinyloxynaphthyl, triazinyloxyanthracenyl, morpholinyloxyphenyl, morpholinyloxynaphthyl, morpholinyloxyanthracenyl, dioxanyloxyphenyl, dioxanyloxynaphthyl, dioxanyloxyanthracenyl, tetrahydro-2H-pyranyloxyphenyl, tetrahydro-2H-pyranyloxynaphthyl, tetrahydro-2H-pyranyloxyanthracenyl, 2H-pyranyloxy phenyl, 2H-pyranyloxy naphthyl, 2H-pyranyloxy anthracenyl, 4H-pyranyloxyphenyl, 4H-pyranyloxynaphthyl, 4H-pyranyloxyanthracenyl, thiomorpholinyloxyphenyl, thiomorpholinyloxynaphthyl, thiomorpholinyloxyanthracenyl, quinolinyloxyphenyl, quinolinyloxynaphthyl, quinolinyloxyanthracenyl, fluorenyloxyphenyl, fluorenyloxynaphthyl and fluorenyloxyanthracenyl; and wherein the R^{4j} substituents may be optionally substituted as provided in other embodiments herein.

In another embodiment of the compounds of Formula (III), R⁴ is —R^{4j} or —OR^{4j}; wherein R^{4j} is selected from the group consisting of pyrrolidinylphenyl, pyrrolidinylnaphthyl, pyrrolidinylanthracenyl, pyrrolinylphenyl, pyrrolinyinaphthyl, pyrrolinylanthracenyl, pyrrolylphenyl, pyrrolylnaphthyl, pyrrolylanthracenyl, tetrahydrofuranylphenyl, tetrahydrofuranyinaphthyl, tetrahydrofuranylanthracenyl, furanylphenyl, furanylnaphthyl, furanylanthracenyl, dioxolanylphenyl, dioxolanyinaphthyl, dioxolanylanthracenyl, imidazolidinylphenyl, imidazolidinylnaphthyl, imidazolidinylanthracenyl, imidazolynylphenyl, imidazolynylnaphthyl, imidazolynylanthracenyl, imidazolylphenyl, imidazolyinaphthyl, imidazolylanthracenyl, pyrazolidinylphenyl, pyrazolidinylnaphthyl, pyrazolidinylanthracenyl, pyrazolinylphenyl, pyrazolinylnaphthyl, pyrazolinylanthracenyl, pyrazolylphenyl, pyrazolylnaphthyl, pyrazolylanthracenyl, oxazolylphenyl, oxazolylnaphthyl, oxazolylanthracenyl, isoxazolylphenyl, isoxazolylnaphthyl, isoxazolylanthracenyl, oxadiazolylphenyl, oxadiazolyinaphthyl, oxadiazolylanthracenyl, thiophenylphenyl, thiophenylnaphthyl, thiophenylanthracenyl, thiazolylphenyl, thiazolylnaphthyl, thiazolylanthracenyl, thiadiazolylphenyl, thiadiazolyinaphthyl, thiadiazolylanthracenyl, triazolylphenyl, triazolylnaphthyl, triazolylanthracenyl, piperidinylphenyl, piperidinylnaphthyl, piperidinylanthracenyl, pyridinylphenyl, pyridinyinaphthyl, pyridinylanthracenyl, piperazinylphenyl, piperazinylnaphthyl, piperazinylanthracenyl, pyrazinylphenyl, pyrazinyinaphthyl, pyrazinylanthracenyl, pyrimidinylphenyl, pyrimidinylnaphthyl, pyrimidinylanthracenyl, pyridazinylphenyl, pyridazinylnaphthyl, pyridazinylanthracenyl, triazinylphenyl, triazinylnaphthyl, triazinylanthracenyl, morpholinylphenyl, morpholinylnaphthyl, morpholinylanthracenyl, dioxanylphenyl, dioxanylnaphthyl, dioxanylanthracenyl, tetrahydro-2H-pyranylphenyl, tetrahydro-2H-pyranylnaphthyl, tetrahydro-2H-pyranylanthracenyl, 2H-pyranyl phenyl, 2H-pyranyl naphthyl, 2H-pyranyl anthracenyl, 4H-pyranylphenyl, 4H-pyranylnaphthyl, 4H-pyranylanthracenyl, thiomorpholinylphenyl, thiomorpholinylnaphthyl, thiomorpholinylanthracenyl, quinolinylphenyl, quinolinylnaphthyl, quinolinylanthracenyl, fluorenylphenyl, fluorenylnaphthyl and fluorenylanthracenyl; and wherein the R^{4j} substituents may be optionally substituted as provided in other embodiments herein.

In another embodiment of the compounds of Formula (III), R⁴ is —R^{4j} or —OR^{4j}; wherein R^{4j} is selected from the group consisting of methyl, ethyl, propyl, butyl, cyclobutyl, phenyl, fluorenyl, phenylphenyl, phenylmethyl, phenylethyl, phenylphenylmethyl, diphenylethyl, phenyloxymethyl, phenyloxyethyl, phenyloxyphenyl, naphthyloxymethyl, phenylcyclopropyl, phenylcarbonylphenyl, phenylcarbonylaminoethyl, phenylcarbonylaminoethyl, thiophenylmethyl, phenyl-oxadiazolyl, thiazolylphenyl, phenylthiazolyl, phenylpyridinyl, phenylpyrimidinyl, pyridinylphenyl and pyrimidinylphenyl; and wherein the R^{4j} substituents may be optionally substituted as provided in other embodiments herein.

In another embodiment of the compounds of Formula (III), R⁴ is selected from the group consisting of —R^{4j}, —OR^{4j} and —NR^{4j}R^{4k}; wherein R^{4j} and R^{4k} are independently selected from the group consisting of:

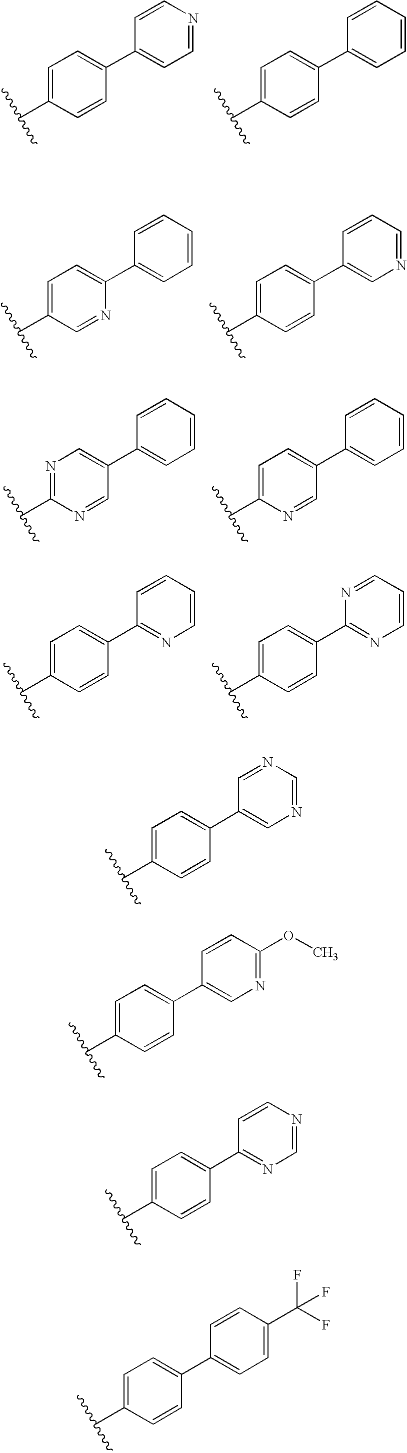

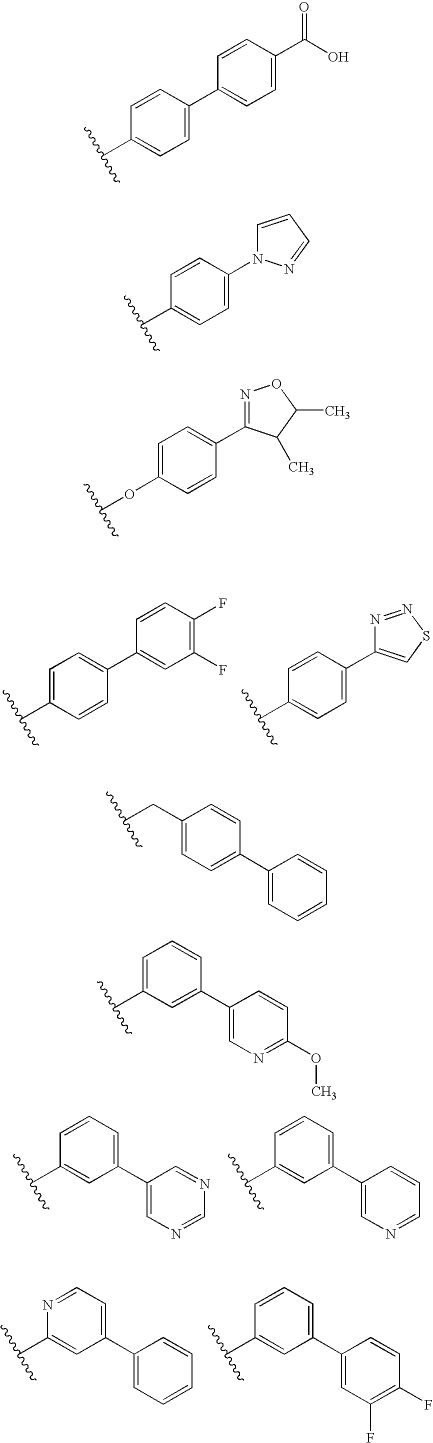

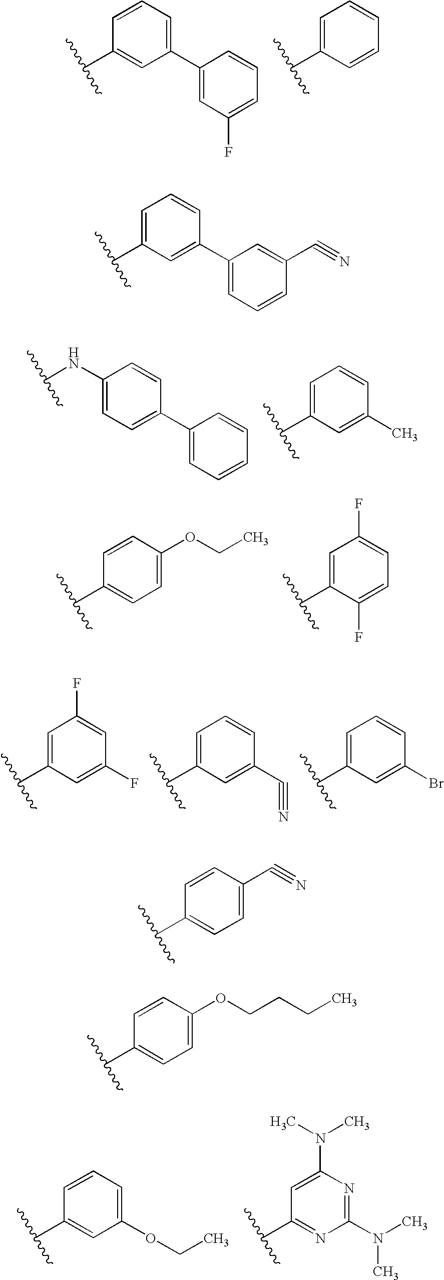

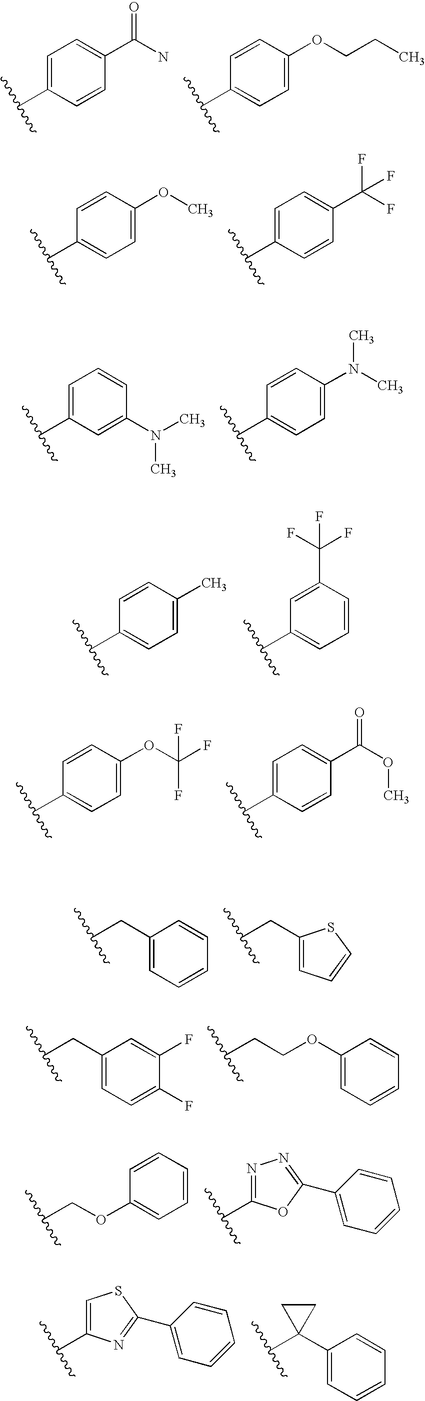

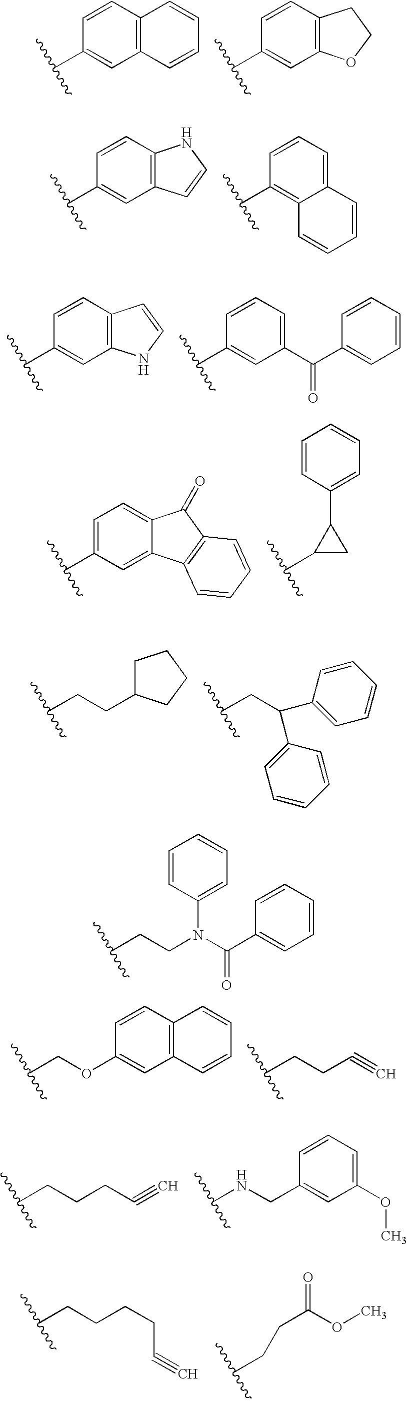

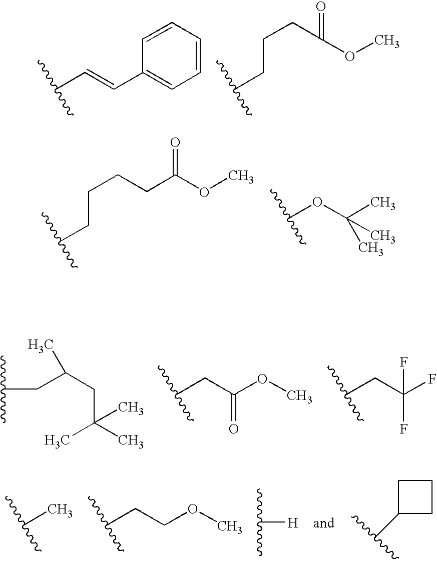

wherein the R^{4j} and R^{4k} substituents may be optionally substituted as provided in other embodiments herein.

In another embodiment of the compounds of Formula (III), the R^{4j} and R^{4k} substituents each may be optionally substituted with one or more substituents independently selected from the group consisting of oxo, cyano, chloro, bromo, fluoro, methyl, ethyl, propyl, butyl, phenyl, methoxy, trifluoromethyl, trifluoromethylmethyl, trifluoromethoxy, ethoxy, propoxy, butoxy, dimethylamino, carboxy, methoxycarbonyl and aminocarbonyl.

Another class of compounds of specific interest includes compounds, and pharmaceutically acceptable salts of the compounds, wherein the compounds have the structure of Formula IV:

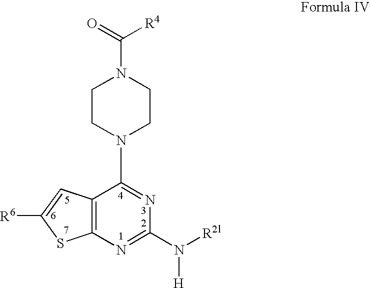

wherein R^{2l} is selected from the group consisting of alkyl, alkenyl, alkynyl, cycloalkyl, aryl and heterocyclyl; wherein:
(a) the R^{2l} alkenyl, alkynyl, cycloalkyl, aryl and heterocyclyl substituents may be optionally substituted with one or more substituents independently selected from the group consisting of halogen and —R^{2m};
(b) the R^{2l} alkyl substituent is substituted with one or more one substituents independently selected from the group consisting of chloro, bromo, iodo and —R^{2m};
R^{2m} is selected from the group consisting of cyano, nitro, amino, alkyl, alkenyl, alkynyl, cycloalkyl, aryl, heterocyclyl, —C(O)R²ⁿ, —C(S)R²ⁿ, —C(O)OR²ⁿ, —C(S)OR²ⁿ, —C(O)SR²ⁿ, —C(O)NR²ⁿR^{2o}, —C(S)NR²ⁿR^{2o},
—OR²ⁿ, —OC(O)R²ⁿ, —OC(S)R²ⁿ, —NR²ⁿR^{2o}, —NR²ⁿC(O)R^{2o}, —NR²ⁿC(S)R^{2o}, —NR²ⁿC(O)OR^{2o}, —NR²ⁿC(S)OR^{2o}, —NR²ⁿS(O)₂R^{2o}, —NR²ⁿC(O)NR^{2o}R^{2p}, —S(O)_{q}R²ⁿ, —S(O)₂NR²ⁿR^{2o} and —SC(O)R²ⁿ;
q is 0, 1 or 2;
R²ⁿ, R^{2o} and R^{2p} are independently selected from the group consisting of hydrogen, alkyl, alkenyl, alkynyl, cycloalkyl, aryl and heterocyclyl;
   wherein the R^{2m}, R²ⁿ, R^{2o} and R^{2p} alkyl, alkenyl, alkynyl, cycloalkyl, aryl, heterocyclyl substituents may be optionally substituted with one or more substituents independently selected from the group consisting of halogen, cyano, nitro, oxo, ═S, —R^{2q}, C(O)R^{2q}, —C(S)R^{2q}, —C(O)OR^{2q}, —C(S)OR^{2q}, —C(O)SR^{2q}, —C(O)NR^{2q}R^{2r}, —C(S)NR^{2q}R^{2r}, —OR^{2q}, —OC(O)R^{2r}, —OC(S)R^{2q}, —NR^{2q}R^{2r}, —NR^{2q}C(O)R^{2r}, —NR^{2q}C(S)R^{2r}, —NR^{2q}C(O)OR^{2r}, —NR^{2q}C(S)OR^{2r}, —NR^{2q}S(O)₂R^{2r}, —NR^{2q}C(O)NR^{2r}R^{2s}, —S(_{O})ᵣR^{2q}, —S(O)₂NR^{2q}R^{2r} and
—SC(O)R²;
r is 0, 1 or 2;
R^{2q}, R^{2r} and R^{2s} are independently selected from the group consisting of hydrogen, alkyl, alkenyl, alkynyl, cycloalkyl, aryl and heterocyclyl;
wherein the R^{2q}, R^{2r} and R^{2s} alkyl, alkenyl, alkynyl, cycloalkyl, aryl and heterocyclyl substituents may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl;
R⁴ is selected from the group consisting of —R^{4j}, —OR^{4j}, and —NR^{4j}R^{4k};
wherein R^{4j} and R^{4k} are independently selected from the group consisting of hydrogen, alkyl, alkenyl, alkynyl, cycloalkyl, aryl, heterocyclyl, cycloalkylalkyl, arylalkyl, heterocyclylalkyl, arylcycloalkyl, heterocyclylcycloalkyl, cycloalkylaryl, cycloalkylheterocyclyl, arylaryl, heterocyclylheterocyclyl, arylheterocyclyl, heterocyclylaryl, cycloalkoxyalkyl, heterocyclyloxyalkyl, aryloxyaryl, heterocyclyloxyheterocyclyl, aryloxyheterocyclyl, heterocyclyloxyaryl, arylcarbonylaryl, heterocyclylcarbonylheterocyclyl, aryloxyalkyl, arylcarbonylheterocyclyl, heterocyclylcarbonylaryl, arylcarbonylaminoalkyl, heterocyclylcarbonylaminoalkyl, arylcarbonylaminoalkyl, and heterocyclylcarbonylaminoalkyl;
wherein the R^{4j} and R^{4k} substituents may be optionally substituted with one or more substituents independently selected from the group consisting of halogen, haloalkyl, hydroxyalkyl, oxo, ═S, nitro, cyano, —R^{4l}, —OR^{4l}, —C(O)R^{4l}, —C(O)OR^{4l}, —C(O)NR^{4l}R^{4m}, —OC(O)R^{4l}, —ONR^{4l}R^{4m}, —NR^{4l}R^{4m},
—NR^{4l}C(O)R^{4m}, —NR^{4l}S(O)₂R^{4m}, —S(O)_{b}R^{4l}, —SC(O)R^{4l} and —SC(O)NR^{4l}R^{4m};
b is 0, 1 or 2;
R^{4l} and R^{4m} are independently selected from the group consisting of hydrogen, alkyl, haloalkyl, alkenyl, cycloalkyl, aryl and heterocyclyl;
R⁶ is selected from the group consisting of halogen, cyano, —R^{6a} and —OR^{6a};
R^{6a} is selected from the group consisting of hydrogen, alkyl, cycloalkyl and aryl; and
wherein the R^{6a} alkyl, cycloalkyl and aryl substituent may be optionally substituted with one or more substituents independently selected from the group consisting of halogen, oxo, ═S, cyano, alkyl, haloalkyl, hydroxyalkyl, cycloalkyl, carboxy,aryl and heterocyclyl.

In another embodiment of the compounds of Formula (IV), R^{2l} is selected from the group consisting of alkyl, cycloalkyl and heterocyclyl; wherein:
(a) the R^{2l} cycloalkyl and heterocyclyl substituents may be optionally substituted with one or more substituents independently selected from the group consisting of halogen and —R^{2m}; and
(b) the R^{2l} alkyl substituent is substituted with one or more one substituents independently selected from,the group consisting of chloro, bromo, iodo and —R^{2m};
R^{2m} is selected from the group consisting of oxo, cyano, nitro, amino, alkyl, cycloalkyl, aryl, heterocyclyl, —C(O)R²ⁿ, —C(S)R²ⁿ, —C(O)OR²ⁿ, —C(S)OR²ⁿ, —C(O)SR²ⁿ, —C(O)NR²ⁿR^{2o}, —C(S)NR²ⁿR^{2o},
—OR²ⁿ, —OC(O)R²ⁿ, —OC(S)²ⁿ, —OC(O)OR²ⁿ, —OC(O)N R²ⁿR^{2o}, —OC(S)NR²ⁿR^{2o}, —N R²ⁿR^{2o}, —NR²ⁿC(O)R^{2o},
—NR²ⁿC(S)R^{2o}, —NR²ⁿC(O)OR^{2o}, —NR²ⁿC(S)OR^{2o}, —NR²ⁿS(O)₂R^{2o}, —NR²ⁿC(O)NR^{2o}R^{2p}, —S(O)_{q}R²ⁿ, —S(O)₂NR²ⁿR^{2o} and —SC(O)R²ⁿ; q is 0, 1 or 2; R²ⁿ, R^{2o} and R^{2p} are independently selected from the group consisting of hydrogen, alkyl, cycloalkyl, aryl and heterocyclyl; wherein the R^{2m}, R²ⁿ, R^{2o} and R^{2p} alkyl, cycloalkyl, aryl, heterocyclyl substituents may be optionally substituted as provided in other embodiments herein.

In another embodiment of the compounds of Formula (IV), R^{2l} is selected from the group consisting of alkyl, cycloalkyl and heterocyclyl; wherein:
(a) the R^{2l} cycloalkyl and heterocyclyl substituents may be optionally substituted with one or more substituents independently selected from the group consisting of halogen and —R^{2m}; and
(b) the R^{2l} alkyl substituent is substituted with one or more one substituents independently selected from the group consisting of chloro, bromo, iodo and —R^{2m};

R^{2m} is selected from the group consisting of oxo, alkyl, cycloalkyl, aryl, heterocyclyl, —C(O)OR²ⁿ, —C(O)NR²ⁿR^{2o}, —OR²ⁿ, and —NR²ⁿR^{2o}; R²ⁿ, R^{2o} and R^{2p} are independently selected from the group consisting of hydrogen, alkyl, cycloalkyl, aryl and heterocyclyl; wherein the R^{2m}, R²ⁿ, R^{2o} and R^{2p} alkyl, cycloalkyl, aryl, heterocyclyl substituents may be optionally substituted as provided in other embodiments herein.

In another embodiment of the compounds of Formula (IV), R^{2l} is cycloalkyl; wherein the R^{2l} cycloalkyl substituent may be optionally substituted with one or more substituents independently selected from the group consisting of halogen and —R^{2m}; and R^{2m} is selected from the group consisting of oxo, cyano, nitro, amino, alkyl, cycloalkyl, aryl, heterocyclyl, —C(O)R²ⁿ, —C(S)R²ⁿ, —C(O)OR²ⁿ, —C(S)OR²ⁿ, —C(O)SR²ⁿ, —C(O)NR²ⁿR^{2o}, —C(S)NR²ⁿR^{2o}, —OR²ⁿ, —OC(O)R²ⁿ, —OC(S)R²ⁿ, —OC(O)OR²ⁿ, —OC(O)NR²ⁿR^{2o}, —OC(S)NR²ⁿR^{2o}, —NR²ⁿR^{2o}, —NR²ⁿC(O)R^{2o}, —NR²ⁿC(S)R^{2o}, —NR²ⁿC(O)OR^{2o}, —NR²ⁿC(S)OR^{2o}, —NR²ⁿS(O)₂R^{2o}, —NR²ⁿC(O)NR^{2o}R^{2p}, S(O)_{q}R²ⁿ, —S(O)₂NR²ⁿR^{2o} and —SC(O)R²ⁿ; q is 0, 1 or 2; R²ⁿ, R^{2o} and R^{2p} are independently selected from the group consisting of hydrogen, alkyl, cycloalkyl, aryl and heterocyclyl; wherein the R^{2m}, R²ⁿ, R^{2o} and R^{2p} alkyl, cycloalkyl, aryl, heterocyclyl substituents may be optionally substituted as provided in other embodiments herein.

In another embodiment of the compounds of Formula (IV), R^{2l} is cycloalkyl; wherein the R^{2l} cycloalkyl substituent may be optionally substituted with one or more substituents independently selected from the group consisting of halogen and —R^{2m}; and R^{2m} is selected from the group consisting of oxo, alkyl, cycloalkyl, aryl, heterocyclyl, —C(O)OR²ⁿ, —C(O)NR²ⁿR^{2o}, —OR²ⁿ, and —NR²ⁿR^{2o}; R²ⁿ and R^{2o} are independently selected from the group consisting of hydrogen, alkyl, cycloalkyl, aryl and heterocyclyl; wherein the R^{2m}, R²ⁿ and R^{2o} alkyl, cycloalkyl, aryl, heterocyclyl substituents may be optionally substituted as provided in other embodiments herein.

In another embodiment of the compounds of Formula (IV), R^{2l} is cycloalkyl; wherein the R^{2l} cycloalkyl substituent may be optionally substituted with one or more substituents independently selected from the group consisting of halogen and —R^{2m}; and R^{2m} is selected from the group consisting of oxo, alkyl, cycloalkyl, aryl, heterocyclyl, —C(O)OR²ⁿ, —C(O)NR²ⁿR^{2o}, —OR²ⁿ, and —NR²ⁿR^{2o}; R²ⁿ and R^{2o} are independently selected from the group consisting of hydrogen and alkyl; wherein the R^{2m}, R²ⁿ and R^{2o} alkyl substituents may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl.

In another embodiment of the compounds of Formula (IV), R^{2l} is heterocyclyl; wherein the R^{2l} heterocyclyl substituent may be optionally substituted with one or more substituents independently selected from the group consisting of halogen and —R^{2m}; and R^{2m} is selected from the group consisting of oxo, cyano, nitro, amino, —SR²ⁿ, alkyl, aryl, heterocyclyl, —C(O)OR²ⁿ, —C(O)NR²ⁿR^{2o}, —OR²ⁿ, and —NR²ⁿR^{2o}; R²ⁿ and R^{2o} are independently selected from the group consisting of hydrogen, alkyl, cycloalkyl, aryl and heterocyclyl; wherein the R^{2m}, R²ⁿ and R^{2o} are alkyl, cycloalkyl, aryl, heterocyclyl substituents may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl.

In another embodiment of the compounds of Formula (IV), R^{2l} is heterocyclyl; wherein the R^{2l} heterocyclyl substituent may be optionally substituted with one or more substituents independently selected from the group consisting of halogen and —R^{2m}; and R^{2m} is selected from the group consisting of oxo and hydroxy.

In another embodiment of the compounds of Formula (IV), R^{2l} is alkyl; wherein the R^{2l} alkyl substituent is substituted with one or more one substituents independently selected from the group consisting of chloro, bromo, iodo and —R^{2m}; R^{2m} is selected from the group consisting of oxo, cyano, nitro, amino, alkyl, cycloalkyl, aryl, heterocyclyl, —C(O)R²ⁿ, —C(S)R²ⁿ, —C(O)OR²ⁿ, C(S)OR²ⁿ, C(O)SR²ⁿ,
—C(O)NR²ⁿR^{2o}, —C(S)NR²ⁿR^{2o}, —OR²ⁿ, —OC(O)R²ⁿ, —OC(S)R²ⁿ, —OC(O)OR²ⁿ, —OC(O)NR²ⁿR^{2o}, —OC(S)NR²ⁿR^{2o}, —NR²ⁿR^{2o}, —NR²ⁿC(O)R^{2o}, —NR²ⁿC(S)R^{2o}, —NR²ⁿC(O)OR^{2o}, —NR²ⁿC(S)OR^{2o}, —NR²ⁿS(O)₂R^{2o}, —NR²ⁿC(O)NR^{2o}R^{2p}p, —S(O)_{q}R²ⁿ, —S(O)₂NR²ⁿR^{2o} and —SC(O)R²ⁿ; q is 0, 1 or 2; R²ⁿ, R^{2o} and R^{2p} are independently selected from the group consisting of hydrogen, alkyl, cycloalkyl, aryl and heterocyclyl; wherein the R^{2m}, R²ⁿ, R^{2o} and R^{2p} alkyl, cycloalkyl, aryl, heterocyclyl substituents may be optionally substi,tuted as provided in other embodiments herein. In another embodiment of the compounds of Formula (IV), R^{2l} is alkyl; wherein the R^{2l} alkyl substituent is substituted with one or more one substituents independently selected from the group consisting of chloro, bromo, iodo and —R^{2m}; R^{2m} is selected from the group consisting of oxo, alkyl, cycloalkyl, aryl, heterocyclyl, —C(O)OR²ⁿ, —C(O)NR²ⁿR^{2o}, —OR²ⁿ, and —NR²ⁿR^{2o}; R²ⁿ and R^{2o} are independently selected from the group consisting of hydrogen, alkyl, cycloalkyl, aryl and heterocyclyl; wherein the R^{2m}, R²ⁿ and R^{2o} alkyl, cycloalkyl, aryl, heterocyclyl substituents may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl.

In another embodiment of the compounds of Formula (IV), R^{2l} is selected from the group consisting of cycloalkyl, arylalkyl, heterocyclylalkyl, hydroxyalkyl, haloarylalkyl, alkoxyalkyl, oxoalkyl, carboxyalkyl, hydroxyalkylaminocarbonylalkyl, alkylaminoalkyl, aminoalkyl, aminocarbonylalkyl, alkylaminocarbonylalkyl, carboxycycloalkyl, aminocarbonylcycloalkyl, hydroxyheterocyclyl, hydroxycycloalkyl, hydroxyalkoxycycloalkyl, aminocarbonylcycloalkyl, hydroxyalkoxyalkyl, carboxyalkoxyalkyl, aminocarbonylalkoxyalkyl, carboxyalkylaminoalkyl, oxoheterocyclyl, heterocyclyl, hydroxyalkylaminoalkyl and aminoalkoxyalkyl; and wherein the R^{2l} substituents may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl.

In another embodiment of the compounds of Formula (IV), R^{2l} is selected from the group consisting of cycloalkyl, arylalkyl, heterocyclylalkyl, hydroxyalkyl, haloarylalkyl, alkoxyalkyl, oxoalkyl, carboxyalkyl, hydroxyalkylaminocarbonylalkyl and alkylaminoalkyl; and wherein the R^{2l} substituents may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl.

In another embodiment of the compounds of Formula (IV), R^{2l} is selected from the group consisting of cyclopropyl, cyclobutyl, cyclopentyl, cyclohexyl, phenylmethyl, phenylethyl, phenylpropyl, phenylbutyl, phenylpentyl, phenylhexyl, hydroxymethyl, hydroxyethyl, hydroxypropyl, hydroxybutyl, hydroxypentyl and hydroxyhexyl; and wherein the R^{2l} substituents may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl.

In another embodiment of the compounds of Formula (IV), R^{2l} is selected from the group consisting of fluorophenylmethyl, fluorophenylethyl, fluorophenylpropyl, fluorophenylbutyl, fluorophenylpentyl, fluorophenylhexyl, chlorophenylmethyl, chlorophenylethyl, chlorophenylpropyl, chlorophenylbutyl, chlorophenylpentyl, chlorophenylhexyl, bromophenylmethyl, bromophenylethyl, bromophenylpropyl, bromophenylbutyl, bromophenylpentyl, bromophenylhexyl, iodophenylmethyl, iodophenylethyl, iodophenylpropyl, iodophenylbutyl, iodophenylpentyl and iodophenylhexyl; and wherein the R^{2l} substituents may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl.

In another embodiment of the compounds of Formula (IV), R^{2l} is selected from the group consisting of oxomethyl, oxoethyl, oxopropyl, oxobutyl, oxopentyl, oxohexyl, carboxymethyl, carboxyethyl, carboxypropyl, carboxybutyl, carboxypentyl, carboxyhexyl, methoxymethyl, methoxyethyl, methoxypropyl, methoxybutyl, methoxypentyl, methoxyhexyl, ethoxymethyl, ethoxyethyl, ethoxypropyl, ethoxybutyl, ethoxypentyl, ethoxyhexyl, propoxymethyl, propoxyethyl, propoxypropyl, propoxybutyl, propoxypentyl, propoxyhexyl, butoxymethyl, butoxyethyl, butoxypropyl, butoxybutyl, butoxypentyl and butoxyhexyl; and wherein the R^{2l} substituents may be optionally substituted with one or more substituents independently selected from the group consisting of halogen, hydroxy, cyano, oxo, ═S, —SH, nitro, alkyl, haloalkyi, hydroxyalkyl, carboxy, alkoxy and alkoxycarbonyl.

In another embodiment of the compounds of Formula (IV), R^{2l} is selected from the group consisting of methylaminomethyl, methylaminoethyl, methylaminopropyl, methylaminobutyl, methylaminopentyl, methylaminohexyl, ethylaminomethyl, ethylaminoethyl, ethylaminopropyl, ethylaminobutyl, ethylaminopentyl, ethylaminohexyl, propylaminomethyl, propylaminoethyl, propylaminopropyl, propylaminobutyl, propylaminopentyl, propylaminohexyl, butylaminomethyl, butylaminoethyl, butylaminopropyl, butylaminobutyl, butylaminopentyl, butylaminohexyl, hydroxymethylaminocarbonylmethyl, hydroxymethylaminocarbonylethyl, hydroxymethylaminocarbonylpropyl, hydroxymethylaminocarbonylbutyl, hydroxyethylaminocarbonylmethyl, hydroxyethylaminocarbonylethyl, hydroxyethylaminocarbonylpropyl, hydroxyethylaminocarbonylbutyl, hydroxypropylaminocarbonylmethyl, hydroxypropylaminocarbonylethyl, hydroxypropylaminocarbonylpropyl, hydroxypropylaminocarbonylbutyl, hydroxybutylaminocarbonylmethyl, hydroxybutylaminocarbonylethyl, hydroxybutylaminocarbonylpropyl and hydroxybutylaminocarbonylbutyl; and wherein the R^{2l} substituents may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl.

In another embodiment of the compounds of Formula (IV), R^{2l} is selected from the group consisting of pyrrolidinylmethyl, pyrrolidinylethyl, pyrrolidinylpropyl, pyrrolidinylbutyl, pyrrolinylmethyl, pyrrolinylethyl, pyrrolinylpropyl, pyrrolinylbutyl, pyrrolylmethyl, pyrrolyiethyl, pyrrolylpropyl, pyrrolylbutyl, tetrahydrofuranylmethyl, tetrahydrofuranylethyl, tetrahydrofuranylpropyl, tetrahydrofuranylbutyl, furanylmethyl, furanylethyl, furanylpropyl, furanylbutyl, dioxolanylmethyl, dioxolanylethyl, dioxolanylpropyl, dioxolanylbutyl, imidazolidinylmethyl, imidazolidinylethyl, imidazolidinylpropyl, imidazolidinylbutyl, imidazolynylmethyl, imidazolynylethyl, imidazolynylpropyl, imidazolynylbutyl, imidazolylmethyl, imidazolylethyl, imidazolylpropyl, imidazolylbutyl, pyrazolidinylmethyl, pyrazolidinylethyl, pyrazolidinylpropyl, pyrazolidinylbutyl, pyrazolinylmethyl, pyrazolinylethyl, pyrazolinylpropyl, pyrazolinylbutyl, pyrazolylmethyl, pyrazolylethyl, pyrazolylpropyl, pyrazolylbutyl, oxazolylmethyl, oxazolylethyl, oxazolylpropyl, oxazolylbutyl, isoxazolylmethyl,-isoxazolylethyl, isoxazolylpropyl, isoxazolylbutyl, 1,2,3-oxadiazolylmethyl, 1,2,3-oxadiazolylethyl, 1,2,3-oxadiazolylpropyl, 1,2,3-oxadiazolylbutyl, 1,3,4-oxadiazolylmethyl, 1,3,4-oxadiazolylethyl, 1,3,4-oxadiazolylpropyl, 1,3,4-oxadiazolylbutyl, thiophenylmethyl, thiophenylethyl, thiophenylpropyl, thiophenylbutyl, thiazolylmethyl, thiazolylethyl, thiazolylpropyl, thiazolylbutyl, thiadiazolylmethyl, thiadiazolylethyl, thiadiazolylpropyl, thiadiazolylbutyl, triazolylmethyl, triazolyiethyl, triazolylpropyl, triazolylbutyl, piperidinylmethyl, piperidinylethyl, piperidinylpropyl, piperidinylbutyl, pyridinylmethyl, pyridinylethyl, pyridinylpropyl, pyridinylbutyl, piperazinylmethyl, piperazinylethyl, piperazinylpropyl, piperazinylbutyl, pyrazinylmethyl, pyrazinylethyl, pyrazinylpropyl, pyrazinylbutyl, pyrimidinylmethyl, pyrimidinylethyl, pyrimidinylpropyl, pyrimidinylbutyl, pyridazinylmethyl, pyridazinylethyl, pyridazinylpropyl, pyridazinylbutyl, triazinylmethyl, triazinylethyl, triazinylpropyl, triazinylbutyl, morpholinylmethyl, morpholinylethyl, morpholinylpropyl, morpholinylbutyl, dioxanylmethyl, dioxanylethyl, dioxanylpropyl, dioxanylbutyl, tetrahydro-2H-pyranylmethyl, tetrahydro-2H-pyranylethyl, tetrahydro-2H-pyranylpropyl, tetrahydro-2H-pyranylbutyl, 2H-pyranylmethyl, 2H-pyranylethyl, 2H-pyranylpropyl, 2H-pyranylbutyl, 4H-pyranylmethyl, 4H-pyranylethyl, 4H-pyranylpropyl, 4H-pyranylbutyl, thiomorpholinylmethyl, thiomorpholinylethyl, thiomorpholinylpropyl, thiomorpholinylbutyl, quinolinylmethyl, quinolinylethyl, quinolinylpropyl, quinolinylbutyl, fluorenylmethyl, fluorenylethyl, fluorenylpropyl, fluorenylbutyl, tetrahydrofurodioxolylmethyl, tetrahydrofurodioxolylethyl, tetrahydrofurodioxolylpropyl and tetrahydrofurodioxolylbutyl; and wherein the R^{2l} substituents may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl.

In another embodiment of the compounds of Formula (IV), R^{2l} is selected from the group consisting of phenylpropyl, furanylmethyl, hydroxypropyl, cyclohexyl, hydroxyethyl, cyclopentyl, fluorophenylmethyl, ethoxypropyl, oxopropyl, carboxyethyl, hydroxyethylaminocarbonylethyl, phenylmethyl, hydroxypropyl, methylaminoethyl and hydroxyethylmethyl; and wherein the R^{2l} substituents may be optionally substituted with one or more substituents independently selected from the group consisting of halogen, hydroxy, cyano, oxo, ═S, nitro, —SH, amino, alkyl, haloalkyl, hydroxyalkyl, carboxy, alkoxy, alkoxycarbonyl and alkylamino.

In another embodiment of the compounds of Formula (IV), R^{2l} is selected from the group consisting of cycloalkyl, arylalkyl, heterocyclylalkyl, hydroxyalkyl, haloarylalkyl, alkoxyalkyl, oxoalkyl, carboxyalkyl, hydroxyalkylaminocarbonylalkyl, alkylaminoalkyl, aminoalkyl, aminocarbonylalkyl, alkylaminocarbonylalkyl, carboxycycloalkyl, aminocarbonylcycloalkyl, hydroxyheterocyclyl, hydroxycycloalkyl, hydroxyalkoxycycloalkyl, aminocarbonylcycloalkyl, hydroxyalkoxyalkyl, carboxyalkoxyalkyl, aminocarbonylalkoxyalkyl, carboxyalkylaminoalkyl, oxoheterocyclyl, heterocyclyl, hydroxyalkylaminoalkyl and aminoalkoxyalkyl; wherein the R^{2l} substituents may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; R⁴ is —R^{4j} or —OR^{4j}; wherein R^{4j} is selected from the group consisting of alkyl, alkenyl, alkynyl, cycloalkyl, aryl, heterocyclyl, arylaryl, arylalkyl, heterocyclylalkyl, arylcycloalkyl, cycloalkylaryl, arylheterocyclyl, aryloxyaryl, heterocyclyloxyaryl, arylcarbonylaryl, and arylcarbonylaminoalkyl; wherein the R^{4j} substituents each may be optionally substituted with one or more substituents independently selected from the group consisting of oxo, cyano, halogen, alkyl, phenyl, alkoxy, haloalkyl, haloalkoxy, alkylamino, carboxy, alkoxycarbonyl, and aminocarbonyl; and R⁶ is hydrogen, cyano, halogen, alkyl and haloalkyl.

In another embodiment of the compounds of Formula (IV), R^{2l} is selected from the group consisting of cycloalkyl, arylalkyl, heterocyclylalkyl, hydroxyalkyl, haloarylalkyl, alkoxyalkyl, oxoalkyl, carboxyalkyl, hydroxyalkylaminocarbonylalkyl and alkylaminoalkyl; wherein the R^{2l} substituents may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; R⁴ is —R^{4j}; wherein R^{4j} is selected from the group consisting of alkyl, alkenyl, alkynyl, cycloalkyl, aryl, heterocyclyl, arylaryl, arylalkyl, heterocyclylalkyl, arylcycloalkyl, cycloalkylaryl, arylheterocyclyl, aryloxyaryl, heterocyclyloxyaryl, arylcarbonylaryl, and arylcarbonylaminoalkyl; wherein the R^{4j} substituents each may be optionally substituted with one or more substituents independently selected from the group consisting of oxo, cyano, halogen, alkyl, phenyl, alkoxy, haloalkyl, haloalkoxy, alkylamino, carboxy, alkoxycarbonyl, aminocarbonyl, trifluoromethyl and trifluoromethylmethyl; and R⁶ is selected from the group consisting of hydrogen, halogen, cyano, alkyl and haloalkyl. In still another embodiment, R^{2l} is cycloalkyl. In still another embodiment, R^{2l} is arylalkyl. In still another embodiment, R^{2l} is heterocyclylalkyl. In still another embodiment, R^{2l} is hydroxyalkyl. In still another embodiment, R^{2l} is haloarylalkyl. In still another embodiment, R^{2l} is alkoxyalkyl. In still another embodiment, R^{2l} is oxoalkyl. In still another embodiment, R^{2l} is carboxyalkyl. In still another embodiment, R^{2l} is hydroxyalkylaminocarbonylalkyl. In still another embodiment, R^{2l} is alkylaminoalkyl.

In another embodiment of the compounds of Formula (IV), R^{4j} is phenylphenyl. In still another embodiment, R^{4j} is phenylmethyl. In still another embodiment, R^{4j} is phenylphenylmethyl.

In another embodiment of the compounds of Formula (IV), R^{2l} is cycloalkyl and R^{4j} is phenylphenyl; wherein the R^{2l} cycloalkyl may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; wherein the R^{4j} substituent each may be optionally substituted with one or more substituents independently selected from the group consisting of oxo, cyano, halogen, alkyl, phenyl, alkoxy, haloalkyl, haloalkoxy, alkylamino, carboxy, alkoxycarbonyl, aminocarbonyl, trifluoromethyl and trifluoromethylmethyl; and R⁶ is selected from the group consisting of selected from the group consisting of halogen, cyano and alkyl. In still another embodiment, R^{4j} is phenylmethyl. In still another embodiment, R^{4j} is phenylphenylmethyl.

In another embodiment of the compounds of Formula (IV), R^{2l} is arylalkyl and R^{4j} is phenylphenyl; wherein the R^{2l} cycloalkyl may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; wherein the R^{4j} substituent each may be optionally substituted with one or more substituents independently selected from the group consisting of oxo, cyano, halogen, alkyl, phenyl, alkoxy, haloalkyl, haloalkoxy, alkylamino, carboxy, alkoxycarbonyl, aminocarbonyl, trifluoromethyl and trifluoromethylmethyl; and R⁶ is selected from the group consisting of selected from the group consisting of halogen, cyano and alkyl. In still another embodiment, R^{4j} is phenylmethyl. In still another embodiment, R^{4j} is phenylphenylmethyl.

In another embodiment of the compounds of Formula (IV), R^{2l} is heterocyclylalkyl and R^{4j} is phenylphenyl; wherein the R^{2l} cycloalkyl may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; wherein the R^{4j} substituent each may be optionally substituted with one or more substituents independently selected from the group consisting of oxo, cyano, halogen, alkyl, phenyl, alkoxy, haloalkyl, haloalkoxy, alkylamino, carboxy, alkoxycarbonyl, aminocarbonyl, trifluoromethyl and trifluoromethylmethyl; and R⁶ is selected from the group consisting of selected from the group consisting of halogen, cyano and alkyl. In still another embodiment, R^{4j} is phenylmethyl. In still another embodiment, R^{4j} is phenylphenylmethyl.

In another embodiment of the compounds of Formula (IV), R^{2l} is hydroxyalkyl and R^{4j} is phenylphenyl; wherein the R^{2l} cycloalkyl may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; wherein the R^{4j} substituent each may be optionally substituted with one or more substituents independently selected from the group consisting of oxo, cyano, halogen, alkyl, phenyl, alkoxy, haloalkyl, haloalkoxy, alkylamino, carboxy, alkoxycarbonyl, aminocarbonyl, trifluoromethyl and trifluoromethylmethyl; and R⁶ is selected from the group consisting of selected from the group consisting of halogen, cyano and alkyl. In still another embodiment, R^{4j} is phenylmethyl. In still another embodiment, R^{4j} is phenylphenylmethyl.

In another embodiment of the compounds of Formula (IV), R^{2l} is haloarylalkyl and R^{4j} is phenylphenyl; wherein the R^{2l} cycloalkyl may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; wherein the R^{4j} substituent each may be optionally substituted with one or more substituents independently selected from the group consisting of oxo, cyano, halogen, alkyl, phenyl, alkoxy, haloalkyl, haloalkoxy, alkylamino, carboxy, alkoxycarbonyl, aminocarbonyl, trifluoromethyl and trifluoromethylmethyl; and R⁶ is selected from the group consisting of selected from the group consisting of halogen, cyano and alkyl. In still another embodiment, R^{4j} is phenylmethyl. In still another embodiment, R^{4j} is phenylphenylmethyl.

In another embodiment of the compounds of Formula (IV), R^{2l} is alkoxyalkyl and R^{4j} is phenylphenyl; wherein the R^{2l} cycloalkyl may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; wherein the R^{4j} substituent each may be optionally substituted with one or more substituents independently selected from the group consisting of oxo, cyano, halogen, alkyl, phenyl, alkoxy, haloalkyl, haloalkoxy, alkylamino, carboxy, alkoxycarbonyl, aminocarbonyl, trifluoromethyl and trifluoromethylmethyl; and R⁶ is selected from the group consisting of selected from the group consisting of halogen, cyano and alkyl. In still another embodiment, R^{4j} is phenylmethyl. In still another embodiment, R^{4j} is phenylphenylmethyl.

In another embodiment of the compounds of Formula (IV), R^{2l} is oxoalkyl and R^{4j} is phenylphenyl; wherein the R^{2l} cycloalkyl may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; wherein the R^{4j} substituent each may be optionally substituted with one or more substituents independently selected from the group consisting of oxo, cyano, halogen, alkyl, phenyl, alkoxy, haloalkyl, haloalkoxy, alkylamino, carboxy, alkoxycarbonyl, aminocarbonyl, trifluoromethyl and trifluoromethylmethyl; and R⁶ is selected from the group consisting of selected from the group consisting of halogen, cyano and alkyl. In still another embodiment, R^{4j} is phenylmethyl. In still another embodiment, R^{4j} is phenylphenylmethyl.

In another embodiment of the compounds of Formula (IV), R^{2l} is carboxyalkyl and R^{4j} is phenylphenyl; wherein the R^{2l} cycloalkyl may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; wherein the R^{4j} substituent each may be optionally substituted with one or more substituents independently selected from the group consisting of oxo, cyano, halogen, alkyl, phenyl, alkoxy, haloalkyl, haloalkoxy, alkylamino, carboxy, alkoxycarbonyl, aminocarbonyl, trifluoromethyl and trifluoromethylmethyl; and R⁶ is selected from the group consisting of selected from the group consisting of halogen, cyano and alkyl. In still another embodiment, R^{4j} is phenylmethyl. In still another embodiment, R^{4j} is phenylphenylmethyl.

In another embodiment of the compounds of Formula (IV), R^{2l} is hydroxyalkylaminocarbonylalkyl and R^{4j} is phenylphenyl; wherein the R^{2l} cycloalkyl may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; wherein the R^{4j} substituent each may be optionally substituted with one or more substituents independently selected from the group consisting of oxo, cyano, halogen, alkyl, phenyl, alkoxy, haloalkyl, haloalkoxy, alkylamino, carboxy, alkoxycarbonyl, aminocarbonyl, trifluoromethyl and trifluoromethylmethyl; and R⁶ is selected from the group consisting of selected from the group consisting of halogen, cyano and alkyl. In still another embodiment, R^{4j} is phenylmethyl. In still another embodiment, R^{4j} is phenylphenylmethyl.

In another embodiment of the compounds of Formula (IV), R^{2l} is alkylaminoalkyl and R^{4j} is phenylphenyl; wherein the R^{2l} cycloalkyl may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; wherein the R^{4j} substituent each may be optionally substituted with one or more substituents independently selected from the group consisting of oxo, cyano, halogen, alkyl, phenyl, alkoxy, haloalkyl, haloalkoxy, alkylamino, carboxy, alkoxycarbonyl, aminocarbonyl, trifluoromethyl and trifluoromethylmethyl; and R⁶ is selected from the group consisting of selected from the group consisting of halogen, cyano and alkyl. In still another embodiment, R^{4j} is phenylmethyl. In still another embodiment, R^{4j} is phenylphenylmethyl.

In another embodiment of the compounds of Formula (IV), R^{2l} is selected from the group consisting of phenylpropyl, furanylmethyl, hydroxypropyl, cyclohexyl, hydroxyethyl, cyclopentyl, fluorophenylmethyl, ethoxypropyl, oxopropyl, carboxyethyl, hydroxyethylaminocarbonylethyl, phenylmethyl, hydroxypropyl, methylaminoethyl and hydroxyethylmethyl; wherein the R^{2l} substituents may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; R⁴ is —R^{4j}; wherein R^{4j} is selected from the group consisting of R^{4j} is selected from the group consisting of methyl, ethyl, propyl, butyl, cyclopropyl, cyclobutyl, phenyl, fluorenyl, phenylphenyl, phenylmethyl, phenylethyl, phenylphenylmethyl, diphenylethyl, phenyloxymethyl, phenyloxyethyl, phenyloxyphenyl, naphthyloxymethyl, phenylcyclopropyl, phenylcarbonylphenyl, phenylcarbonylaminoethyl, phenylcarbonylaminoethyl, thiophenylmethyl, phenyl-oxadiazolyl, oxadiazolylphenyl, thiazolylphenyl, phenylthiazolyl, phenylpyridinyl, phenylpyrimidinyl, pyridinylphenyl and pyrimidinylphenyl; wherein the R^{4j} substituents each may be optionally substituted with one or more substituents independently selected from the group consisting of oxo, cyano, halogen, alkyl, phenyl, alkoxy, haloalkyl, haloalkoxy, alkylamino, carboxy, alkoxycarbonyl, aminocarbonyl, trifluoromethyl and trifluoromethylmethyl; and R⁶ is selected from the group consisting of hydrogen, methyl, ethyl, propyl, butyl, fluoromethyl, difluoromethyl, trifluoromethyl, trifluoromethylmethyl, fluoroethyl, difluoroethyl and trifluoroethyl.

In another embodiment of the compounds of Formula (IV), R^{2l} is selected from the group consisting of phenylpropyl, furanylmethyl, hydroxypropyl, cyclohexyl, hydroxyethyl, cyclopentyl, fluorophenylmethyl, ethoxypropyl, oxopropyl, carboxyethyl, hydroxyethylaminocarbonylethyl, phenylmethyl, hydroxypropyl, methylaminoethyl and hydroxyethylmethyl; wherein the R^{2l} substituents may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; R⁴ is —R^{4j}; wherein R^{4j} is selected from the group consisting of R^{4j} is selected from the group consisting of methyl, ethyl propyl, butyl, cyclopropyl, cyclobutyl, phenyl, fluorenyl, phenylphenyl, phenyimethyl, phenylethyl, phenylphenylmethyl, diphenylethyl, phenyloxymethyl, phenyloxyethyl, phenyloxyphenyl, naphthyloxymethyl, phenylcyclopropyl, phenylcarbonylphenyl, phenylcarbonylaminoethyl, phenylcarbonylaminoethyl, thiophenylmethyl, phenyl-oxadiazolyl, oxadiazolylphenyl, thiazolylphenyl, phenylthiazolyl, phenylpyridinyl, phenylpyrimidinyl, pyridinylphenyl and pyrimidinylphenyl; wherein the R^{4j} substituents each may be optionally substituted with one or more substituents independently selected from the group consisting of oxo, cyano, chloro, bromo, fluoro, methyl, ethyl, propyl, butyl, phenyl, methoxy, trifluoromethyl, trifluoromethylmethyl, trifluoromethoxy, ethoxy, propoxy, butoxy, dimethylamino, carboxy, methoxycarbonyl and aminocarbonyl; and R⁶ is ethyl.

In another embodiment of the compounds of Formula (IV), R^{2l} is furanylmethyl; wherein the R^{2l} furanylmethyl may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; R⁴ is —R^{4j}; wherein R^{4j} is selected from the group consisting of R^{4j} is selected from the group consisting of methyl, ethyl propyl, butyl, cyclopropyl, cyclobutyl, phenyl, fluorenyl, phenylphenyl, phenylmethyl, phenylethyl, phenylphenylmethyl, diphenylethyl, phenyloxymethyl, phenyloxyethyl, phenyloxyphenyl, naphthyloxymethyl, phenylcyclopropyl, phenylcarbonylphenyl, phenylcarbonylaminoethyl, phenylcarbonylaminoethyl, thiophenylmethyl, phenyl-oxadiazolyl, oxadiazolylphenyl, thiazolylphenyl, phenylthiazolyl, phenylpyridinyl, phenylpyrimidinyl, pyridinylphenyl and pyrimidinylphenyl; wherein the R^{4j} substituents each may be optionally substituted with one or more substituents independently selected from the group consisting of oxo, cyano, chloro, bromo, fluoro, methyl, ethyl, propyl, butyl, phenyl, methoxy, trifluoromethyl, trifluoromethylmethyl, trifluoromethoxy, ethoxy, propoxy, butoxy, dimethylamino, carboxy, methoxycarbonyl and aminocarbonyl; and R⁶ is ethyl.

In another embodiment of the compounds of Formula (IV), R^{2l} is hydroxypropyl; wherein the R^{2l} hydroxypropyl may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; R⁴ is —R^{4j}; wherein R^{4j} is selected from the group consisting of R^{4j} is selected from the group consisting of methyl, ethyl propyl, butyl, cyclopropyl, cyclobutyl, phenyl, fluorenyl, phenylphenyl, phenylmethyl, phenylethyl, phenylphenylmethyl, diphenylethyl, phenyloxymethyl, phenyloxyethyl, phenyloxyphenyl, naphthyloxymethyl, phenylcyclopropyl, phenylcarbonylphenyl, phenylcarbonylaminoethyl, phenylcarbonylaminoethyl, thiophenylmethyl, phenyl-oxadiazolyl, oxadiazolylphenyl, thiazolylphenyl, phenylthiazolyl, phenylpyridinyl, phenylpyrimidinyl, pyridinylphenyl and pyrimidinylphenyl; wherein the R^{4j} substituents each may be optionally substituted with one or more substituents independently selected from the group consisting of oxo, cyano, chloro, bromo, fluoro, methyl, ethyl, propyl, butyl, phenyl, methoxy, trifluoromethyl, trifluoromethylmethyl, trifluoromethoxy, ethoxy, propoxy, butoxy, dimethylamino, carboxy, methoxycarbonyl and aminocarbonyl; and R⁶ is ethyl.

In another embodiment of the compounds of Formula (IV), R^{2l} is cyclohexyl; wherein the R^{2l} cyclohexyl may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; R⁴ is —R^{4j}; wherein R^{4j} is selected from the group consisting of R^{4j} is selected from the group consisting of methyl, ethyl propyl, butyl, cyclopropyl, cyclobutyl, phenyl, fluorenyl, phenylphenyl, phenylmethyl, phenylethyl, phenylphenylmethyl, diphenylethyl, phenyloxymethyl, phenyloxyethyl, phenyloxyphenyl, naphthyloxymethyl, phenylcyclopropyl, phenylcarbonylphenyl, phenylcarbonylaminoethyl, phenylcarbonylaminoethyl, thiophenylmethyl, phenyl-oxadiazolyl, oxadiazolylphenyl, thiazolylphenyl, phenylthiazolyl, phenylpyridinyl, phenylpyrimidinyl, pyridinylphenyl and pyrimidinylphenyl; wherein the R^{4j} substituents each may be optionally substituted with one or more substituents independently selected from the group consisting of oxo, cyano, chloro, bromo, fluoro, methyl, ethyl, propyl, butyl, phenyl, methoxy, trifluoromethyl, trifluoromethylmethyl, trifluoromethoxy, ethoxy, propoxy, butoxy, dimethylamino, carboxy, methoxycarbonyl and aminocarbonyl; and R⁶ is ethyl.

In another embodiment of the compounds of Formula (IV), R^{2l} is hydroxyethyl; wherein the R^{2l} hydroxyethyl may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; R⁴ is —R^{4j}; wherein R^{4j} is selected from the group consisting of R^{4j} is selected from the group consisting of methyl, ethyl propyl, butyl, cyclopropyl, cyclobutyl, phenyl, fluorenyl, phenylphenyl, phenylmethyl, phenylethyl, phenylphenylmethyl, diphenylethyl, phenyloxymethyl, phenyloxyethyl, phenyloxyphenyl, naphthyloxymethyl, phenylcyclopropyl, phenylcarbonylphenyl, phenylcarbonylaminoethyl, phenylcarbonylaminoethyl, thiophenylmethyl, phenyl-oxadiazolyl, oxadiazolylphenyl, thiazolylphenyl, phenylthiazolyl, phenylpyridinyl, phenylpyrimidinyl, pyridinylphenyl and pyrimidinylphenyl; wherein the R^{4j} substituents each may be optionally substituted with one or more substituents independently selected from the group consisting of oxo, cyano, chloro, bromo, fluoro, methyl, ethyl, propyl, butyl, phenyl, methoxy, trifluoromethyl, trifluoromethylmethyl, trifluoromethoxy, ethoxy, propoxy, butoxy, dimethylamino, carboxy, methoxycarbonyl and aminocarbonyl; and R⁶ is ethyl.

In another embodiment of the compounds of Formula (IV), R^{2l} cyclopentyl; wherein the R^{2l} cyclopentyl may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; R⁴ is —R^{4j}; wherein R^{4j} is selected from the group consisting of R^{4j} is selected from the group consisting of methyl, ethyl propyl, butyl, cyclopropyl, cyclobutyl, phenyl, fluorenyl, phenylphenyl, phenylmethyl, phenylethyl, phenylphenylmethyl, diphenylethyl, phenyloxymethyl, phenyloxyethyl, phenyloxyphenyl, naphthyloxymethyl, phenylcyclopropyl, phenylcarbonylphenyl, phenylcarbonylaminoethyl, phenylcarbonylaminoethyl, thiophenylmethyl, phenyl-oxadiazolyl, oxadiazolylphenyl, thiazolylphenyl, phenylthiazolyl, phenylpyridinyl, phenylpyrimidinyl, pyridinylphenyl and pyrimidinylphenyl; wherein the R^{4j} substituents each may be optionally substituted with one or more substituents independently selected from the group consisting of oxo, cyano, chloro, bromo, fluoro, methyl, ethyl, propyl, butyl, phenyl, methoxy, trifluoromethyl, trifluoromethylmethyl, trifluoromethoxy, ethoxy, propoxy, butoxy, dimethylamino, carboxy, methoxycarbonyl and aminocarbonyl; and R⁶ is ethyl.

In another embodiment of the compounds of Formula (IV), R^{2l} is fluorophenylmethyl; wherein the R^{2l} fluorophenylmethyl may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; R⁴ is —R^{4j}; wherein R^{4j} is selected from the group consisting of R^{4j} is selected from the group consisting of methyl, ethyl propyl, butyl, cyclopropyl, cyclobutyl, phenyl, fluorenyl, phenylphenyl, phenylmethyl, phenylethyl, phenylphenylmethyl, diphenylethyl, phenyloxymethyl, phenyloxyethyl, phenyloxyphenyl, naphthyloxymethyl, phenylcyclopropyl, phenylcarbonylphenyl, phenylcarbonylaminoethyl, phenylcarbonylaminoethyl, thiophenylmethyl, phenyl-oxadiazolyl, oxadiazolylphenyl, thiazolylphenyl, phenylthiazolyl, phenylpyridinyl, phenylpyrimidinyl, pyridinylphenyl and pyrimidinylphenyl; wherein the R^{4j} substituents each may be optionally substituted with one or more substituents independently selected from the group consisting of oxo, cyano, chloro, bromo, fluoro, methyl, ethyl, propyl, butyl, phenyl, methoxy, trifluoromethyl, trifluoromethylmethyl, trifluoromethoxy, ethoxy, propoxy, butoxy, dimethylamino, carboxy, methoxycarbonyl and aminocarbonyl; and R⁶ is ethyl.

In another embodiment of the compounds of Formula (IV), R^{2l} is ethoxypropyl; wherein the R^{2l} ethoxypropyl may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; R⁴ is —R^{4j}; wherein R^{4j} is selected from the group consisting of R^{4j} is selected from the group consisting of methyl, ethyl propyl, butyl, cyclopropyl, cyclobutyl, phenyl, fluorenyl, phenylphenyl, phenylmethyl, phenylethyl, phenylphenylmethyl, diphenylethyl, phenyloxymethyl, phenyloxyethyl, phenyloxyphenyl, naphthyloxymethyl, phenylcyclopropyl, phenylcarbonylphenyl, phenylcarbonylaminoethyl, phenylcarbonylaminoethyl, thiophenylmethyl, phenyl-oxadiazolyl, oxadiazolylphenyl, thiazolylphenyl, phenylthiazolyl, phenylpyridinyl, phenylpyrimidinyl, pyridinylphenyl and pyrimidinylphenyl; wherein the R^{4j} substituents each may be optionally substituted with one or more substituents independently selected from the group consisting of oxo, cyano, chloro, bromo, fluoro, methyl, ethyl, propyl, butyl, phenyl, methoxy, trifluoromethyl, trifluoromethylmethyl, trifluoromethoxy, ethoxy, propoxy, butoxy, dimethylamino, carboxy, methoxycarbonyl and aminocarbonyl; and R⁶ is ethyl.

In another embodiment of the compounds of Formula (IV), R^{2l} is carboxyethyl; wherein the R^{2l} carboxyethyl may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; R⁴ is —R^{4j}; wherein R^{4j} is selected from the group consisting of R^{4j} is selected from the group consisting of methyl, ethyl propyl, butyl, cyclopropyl, cyclobutyl, phenyl, fluorenyl, phenylphenyl, phenylmethyl, phenylethyl, phenylphenylmethyl, diphenylethyl, phenyloxymethyl, phenyloxyethyl, phenyloxyphenyl, naphthyloxymethyl, phenylcyclopropyl, phenylcarbonylphenyl, phenylcarbonylaminoethyl, phenylcarbonylaminoethyl, thiophenylmethyl, phenyl-oxadiazolyl, oxadiazolylphenyl, thiazolylphenyl, phenylthiazolyl, phenylpyridinyl, phenylpyrimidinyl, pyridinylphenyl and pyrimidinylphenyl; wherein the R^{4j} substituents each may be optionally substituted with one or more substituents independently selected from the group consisting of oxo, cyano, chloro, bromo, fluoro, methyl, ethyl, propyl, butyl, phenyl, methoxy, trifluoromethyl, trifluoromethylmethyl, trifluoromethoxy, ethoxy, propoxy, butoxy, dimethylamino, carboxy, methoxycarbonyl and aminocarbonyl; and R⁶ is ethyl.

In another embodiment of the compounds of Formula (IV), R^{2l} is hydroxyethylaminocarbonylethyl; wherein the R^{2l} hydroxyethylaminocarbonylethyl may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; R⁴ is —R^{4j}; wherein R^{4j} is selected from the group consisting of R^{4j} is selected from the group consisting of methyl, ethyl propyl, butyl, cyclopropyl, cyclobutyl, phenyl, fluorenyl, phenylphenyl, phenylmethyl, phenylethyl, phenylphenylmethyl, diphenylethyl, phenyloxymethyl, phenyloxyethyl, phenyloxyphenyl, naphthyloxymethyl, phenylcyclopropyl, phenylcarbonylphenyl, phenylcarbonylaminoethyl, phenylcarbonylaminoethyl, thiophenylmethyl, phenyl-oxadiazolyl, oxadiazolylphenyl, thiazolylphenyl, phenylthiazolyl, phenylpyridinyl, phenylpyrimidinyl, pyridinylphenyl and pyrimidinylphenyl; wherein the R^{4j} substituents each may be optionally substituted with one or more substituents independently selected from the group consisting of oxo, cyano, chloro, bromo, fluoro, methyl, ethyl, propyl, butyl, phenyl, methoxy, trifluoromethyl, trifluoromethylmethyl, trifluoromethoxy, ethoxy, propoxy, butoxy, dimethylamino, carboxy, methoxycarbonyl and aminocarbonyl; and R⁶ is ethyl.

In another embodiment of the compounds of Formula (IV), R^{2l} is phenylmethy); wherein the R^{2l} phenylmethyl may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; R⁴ is —R^{4j}; wherein R^{4j} is selected from the group consisting of R^{4j} is selected from the group consisting of methyl, ethyl propyl, butyl, cyclopropyl, cyclobutyl, phenyl, fluorenyl, phenylphenyl, phenylmethyl, phenylethyl, phenylphenylmethyl, diphenylethyl, phenyloxymethyl, phenyloxyethyl, phenyloxyphenyl, naphthyloxymethyl, phenylcyclopropyl, phenylcarbonylphenyl, phenylcarbonylaminoethyl, phenylcarbonylaminoethyl, thiophenylmethyl, phenyl-oxadiazolyl, oxadiazolylphenyl, thiazolylphenyl, phenylthiazolyl, phenylpyridinyl, phenylpyrimidinyl, pyridinylphenyl and pyrimidinylphenyl; wherein the R^{4j} substituents each may be optionally substituted with one or more substituents independently selected from the group consisting of oxo, cyano, chloro, bromo, fluoro, methyl, ethyl, propyl, butyl, phenyl, methoxy, trifluoromethyl, trifluoromethylmethyl, trifluoromethoxy, ethoxy, propoxy, butoxy, dimethylamino, carboxy, methoxycarbonyl and aminocarbonyl; and R⁶ is ethyl.

In another embodiment of the compounds of Formula (IV), R^{2l} is methylaminoethyl; wherein the R^{2l} methylaminoethyl may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; R⁴ is —R^{4j}; wherein R^{4j} is selected from the group consisting of R^{4j} is selected from the group consisting of methyl, ethyl propyl, butyl, cyclopropyl, cyclobutyl, phenyl, fluorenyl, phenylphenyl, phenylmethyl, phenylethyl, phenylphenylmethyl, diphenylethyl, phenyloxymethyl, phenyloxyethyl, phenyloxyphenyl, naphthyloxymethyl, phenylcyclopropyl, phenylcarbonylphenyl, phenylcarbonylaminoethyl, phenylcarbonylaminoethyl, thiophenylmethyl, phenyl-oxadiazolyl, oxadiazolylphenyl, thiazolylphenyl, phenylthiazolyl, phenylpyridinyl, phenylpyrimidinyl, pyridinylphenyl and pyrimidinyiphenyl; wherein the R^{4j} substituents each may be optionally substituted with one or more substituents independently selected from the group consisting of oxo, cyano, chloro, bromo, fluoro, methyl, ethyl, propyl, butyl, phenyl, methoxy, trifluoromethyl, trifluoromethylmethyl, trifluoromethoxy, ethoxy, propoxy, butoxy, dimethylamino, carboxy, methoxycarbonyl and aminocarbonyl; and R⁶ is ethyl.

In another embodiment of the compounds of Formula (IV), R^{2l} is hydroxyethylmethyl; wherein the R^{2l} hydroxyethylmethyl may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; R⁴ is —R^{4j}; wherein R^{4j} is selected from the group consisting of R^{4j} is selected from the group consisting of methyl, ethyl propyl, butyl, cyclopropyl, cyclobutyl, phenyl, fluorenyl, phenylphenyl, phenylmethyl, phenylethyl, phenylphenylmethyl, diphenylethyl, phenyloxymethyl, phenyloxyethyl, phenyloxyphenyl, naphthyloxymethyl, phenylcyclopropyl, phenylcarbonylphenyl, phenylcarbonylaminoethyl, phenylcarbonylaminoethyl, thiophenylmethyl, phenyl-oxadiazolyl, oxadiazolylphenyl, thiazolylphenyl, phenylthiazolyl, phenylpyridinyl, phenylpyrimidinyl, pyridinylphenyl and pyrimidinylphenyl; wherein the R^{4j} substituents each may be optionally substituted with one or more substituents independently selected from the group consisting of oxo, cyano, chloro, bromo, fluoro, methyl, ethyl, propyl, butyl, phenyl, methoxy, trifluoromethyl, trifluoromethylmethyl, trifluoromethoxy, ethoxy, propoxy, butoxy, dimethylamino, carboxy, methoxycarbonyl and aminocarbonyl; and R⁶ is ethyl.

In another embodiment of the compounds of Formula (IV), R^{2l} is phenylpropyl; wherein the R^{2l} phenylpropyl may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; R⁴ is —R^{4j}; wherein R^{4j} is selected from the group consisting of R^{4j} is selected from the group consisting of methyl, ethyl propyl, butyl, cyclopropyl, cyclobutyl, phenyl, fluorenyl, phenylphenyl, phenylmethyl, phenylethyl, phenylphenylmethyl, diphenylethyl, phenyloxymethyl, phenyloxyethyl, phenyloxyphenyl, naphthyloxymethyl, phenylcyclopropyl, phenylcarbonylphenyl, phenylcarbonylaminoethyl, phenylcarbonylaminoethyl, thiophenylmethyl, phenyl-oxadiazolyl, oxadiazolylphenyl, thiazolylphenyl, phenylthiazolyl, phenylpyridinyl, phenylpyrimidinyl, pyridinylphenyl and pyrimidinylphenyl; wherein the R^{4j} substituents each may be optionally substituted with one or more substituents independently selected from the group consisting of oxo, cyano, chloro, bromo, fluoro, methyl, ethyl, propyl, butyl, phenyl, methoxy, trifluoromethyl, trifluoromethylmethyl, trifluoromethoxy, ethoxy, propoxy, butoxy, dimethylamino, carboxy, methoxycarbonyl and aminocarbonyl; and R⁶ is ethyl.

In another embodiment of the compounds of Formula (IV), R^{2l} is selected from the group consisting of phenylpropyl, furanylmethyl, hydroxypropyl, cyclohexyl, hydroxyethyl, cyclopentyl, fluorophenylmethyl, ethoxypropyl, oxypropyl, carboxyethyl, hydroxyethylaminocarbonylethyl, phenylmethyl, hydroxypropyl, methylaminoethyl and hydroxyethylmethyl; wherein the R^{2l} substituents may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; R⁴ is —R^{4j}; wherein R^{4j} is phenylphenyl; and
R⁶ is ethyl.

In another embodiment of the compounds of Formula (IV), R^{2l} is selected from the group consisting of phenylpropyl, furanylmethyl, hydroxypropyl, cyclohexyl, hydroxyethyl, cyclopentyl, fluorophenylmethyl, ethoxypropyl, oxopropyl, carboxyethyl, hydroxyethylaminocarbonylethyl, phenylmethyl, hydroxypropyl, methylaminoethyl and hydroxyethylmethyl; wherein the R^{2l} substituents may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; R⁴ is —R^{4j}; wherein R^{4j} is phenylmethyl; and R⁶ is ethyl.

In another embodiment of the compounds of Formula (IV), R^{2l} is selected from the group consisting of phenylpropyl, furanylmethyl, hydroxypropyl, cyclohexyl, hydroxyethyl, cyclopentyl, fluorophenylmethyl, ethoxypropyl, oxopropyl, carboxyethyl, hydroxyethylaminocarbonylethyl, phenylmethyl, hydroxypropyl, methylaminoethyl and hydroxyethylmethyl; wherein the R^{2l} substituents may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl; R⁴ is —R^{4j}; wherein R^{4j} is phenylphenylmethyl; and R⁶ is ethyl.

Another embodiment of the compounds of Formula (I) is selected from the group consisting of those compounds listed in Table E, presented herein.

Another embodiment of the compounds of Formula (I) is selected from group consisting of: 6-Ethyl-4-[4-(phenylacetyl)piperazin-1-yl]-N-(3-phenylpropyl)thieno[2,3-d]pyrimidin-2-amine; 6-Ethyl-N-(2-furylmethyl)-4-[4-(phenylacetyl)piperazin-1-yl]thieno[2,3-d]pyrimidin-2-amine; 3-({6-Ethyl-4-[4-(phenylacetyl)piperazin-1-yl]thieno[2,3-d]pyrimidin-2-yl}amino)propan-1-ol; N-Cyclohexyl-6-ethyl-4-[4-(phenylacetyl)piperazin-1-yl]thieno[2,3-d]pyrimidin-2-amine; 2-({6-Ethyl-4-[4-(phenylacetyl)piperazin-1-yl]thieno[2,3-d]pyrimidin-2-yl}amino)ethanol; N-Cyclopentyl-6-ethyl-4-[4-(phenylacetyl)piperazin-1-yl]thieno[2,3-d]pyrimidin-2-amine; 6-Ethyl-N-(4-fluorobenzyl)-4-[4-(phenylacetyl)piperazin-1-yl]thieno[2,3-d]pyrimidin-2-amine; 6-Ethyl-N-(3-fluorobenzyl)-4-[4-(phenylacetyl)piperazin-1-yl]thieno[2,3-d]pyrimidin-2-amine; N-benzyl-6-ethyl-4-[4-(phenylacetyl)piperazin-1-yl]thieno[2,3-d]pyrimidin-2-amine; 4-[4-(1,1′-Biphenyl-4-ylcarbonyl)piperazin-1-yl]-N-(3,3-diethoxypropyl)-6-ethylthieno[2,3-d]pyrimidin-2-amine;
N-{4-[4-(1,1′-Biphenyl-4-ylcarbonyl)piperazin-1-yl]-6-ethylthieno[2,3-d]pyrimidin-2-yl}-beta-alanine;
N³-{4-[4-(1,1′-Biphenyl-4-ylcarbonyl)piperazin-1-yl]-6-ethylthieno[2,3-d]pyrimidin-2-yl}-N˜1˜-[2-hydroxy-1-(hydroxymethyl)ethyl]-beta-alaninamide;
(2S)-3-({4-[4-(1,1′-biphenyl-4-ylcarbonyl)piperazin-1-yl]-6-ethylthieno[2,3-d]pyrimidin-2-yl}amino)propane-1,2-diol;
(2R)-3-({4-[4-(1,1′-biphenyl-4-ylcarbonyl)piperazin-1-yl]-6-ethylthieno[2,3-d]pyrimidin-2-yl}amino)propane-1,2-diol;
N′-{4-[4-(1,1′-biphenyl-4-ylcarbonyl)piperazin-1-yl]-6-ethylthieno[2,3-d]pyrimidin-2-yl}-N,N-dimethylethane-1,2-diamine;
(2S)-3-({4-[4-(1,1′-biphenyl-3-ylcarbonyl)piperazin-1-yl]-6-ethylthieno[2,3-d]pyrimidin-2-yl}amino)propane-1,2-diol;
(2R)-3-({4-[4-(1,1′-biphenyl-3-ylcarbonyl)piperazin-1-yl]-6-ethylthieno[2,3-d]pyrimidin-2-yl}amino)propane-1,2-diol;
2-({4-[4-(1,1′-biphenyl-4-ylcarbonyl)piperazin-1-yl]-6-ethylthieno[2,3-d]pyrimidin-2-yl}amino)propane-1,3-diol;
2-({4-[4-(1,1′-biphenyl-3-ylcarbonyl)piperazin-1-yl]-6-ethylthieno[2,3-d]pyrimidin-2-yl}amino)propane-1,3-diol, and
(2R)-3-({6-Ethyl-4-[4-(3,3,3-trifluoropropanoyl)piperazin-1-yl]thieno[2,3-d]pyrimidin-2-yl}amino)propane-1,2-diol.

### C. Isomers

When an asymmetric center is present in a compound of Formulae (I) through (IV) the compound may exist in the form of optical isomers (enantiomers). In one embodiment, the present invention comprises enantiomers and mixtures, including racemic mixtures of the compounds of Formulae (I) through (IV). In another embodiment, for compounds of Formulae (I) through (IV) that contain more than one asymmetric center, the present invention comprises diastereomeric forms (individual diastereomers and mixtures thereof) of compounds. When a compound of Formulae (I) through (IV) contains an alkenyl group or moiety, geometric isomers may arise.

### D. Tautomeric Forms

The present invention comprises the tautomeric forms of compounds of Formulae (I) through (IV). Where structural isomers are interconvertible via a low energy barrier, tautomeric isomerism (‘tautomerism’) can occur. This can take the form of proton tautomerism in compounds of formula I containing, for example, an imino, keto, or oxime group, or so-called valence tautomerism in compounds which contain an aromatic moiety. It follows that a single compound may exhibit more than one type of isomerism. The various ratios of the tautomers in solid and liquid form is dependent on the various substituents on the molecule as well as the particular crystallization technique used to isolate a compound.

### E. E. Salts

The compounds of this invention may be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

Where a salt is intended to be administered to a patient (as opposed to, for example, being used in an in vitro context), the salt may comprise a pharmaceutically acceptable salt. The term “pharmaceutically acceptable salt” refers to a salt prepared by combining a compound of Formulae (I)-(IV) with an acid whose anion, or a base whose cation, is generally considered suitable for human consumption. Pharmaceutically acceptable salts are particularly useful as products of the methods of the present invention because of their greater aqueous solubility relative to the parent compound. For use in medicine, the salts of the compounds of this invention are non-toxic “pharmaceutically acceptable salts.” Salts encompassed within the term “pharmaceutically acceptable salts” refer to non-toxic salts of the compounds of this invention which are generally prepared by reacting the free base with a suitable organic or inorganic acid. Suitable pharmaceutically acceptable acid addition salts of the compounds of the present invention when possible include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. Suitable organic acids generally include, for example, aliphatic, cycloaliphatic, aromatic, araliphatic, heterocyclylic, carboxylic, and sulfonic classes of organic acids. Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, β-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, tosylate, and undecanoate. In another embodiment, examples of suitable addition salts formed include the acetate, aspartate, benzoate, besylate, bicarbonate/carbonate, bisulphate/sulphate, borate, camsyate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, nitrate, orotate, oxalate, palmitate, pamoate, phosphate/hydrogen phosphate/dihidrogen phosphate, saccharate, stearate, succinate, tartrate, tosylate and trifluoroacetate salts. In another embodiment, representative salts include benzenesulfonate, hydrobromide and hydrochloride.

Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. In another embodiment, base salts are formed from bases which form non-toxic salts, including aluminum, arginine, benzathine, choline, diethylamine, diolamine, glycine, lysine, meglumine, olamine, tromethamine and zinc salts.

Organic salts may be made from secondary, tertiary or quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups may be quaternized with agents such as lower alkyl (C₁-C₆) halides (e.g., methyl, ethyl, propyl, and butyl chlorides, bromides, and iodides), dialkyl sulfates (e.g., dimethyl, diethyl, dibuytl, and diamyl sulfates), long chain halides (e.g., decyl, lauryl, myristyl, and stearyl chlorides, bromides, and iodides), arylalkyl halides (e.g., benzyl and phenethyl bromides), and others.

In one embodiment, hemisalts of acids and bases may also be formed, for example, hemisulphate and hemicalcium salts.

### F. Prodrugs

Also within the scope of the present invention are so-called “prodrugs” of the compounds of Formulae (I) through (IV). Thus, certain derivatives of compounds of any of Formulae (I) through (IV) which may have little or no pharmacological activity themselves can, when administered into or onto the body, be converted into compounds of any of Formulae (I) through (IV) having the desired activity, for example, by hydrolytic cleavage. Such derivatives are referred to as “prodrugs.” Further information on the use of prodrugs may be found in “Pro-drugs as Novel Delivery Systems, Vol. 14, ACS Symposium Series (T Higuchi and W Stella) and “Bioreversible Carriers in Drug Design,” Pergamon Press, 1987 (ed. E B Roche, American Pharmaceutical Association). Prodrugs in accordance with the invention can, for example, be produced by replacing appropriate functionalities present in the compounds of any of Formulae (I) through (IV) with certain moieties known to those skilled in the art as “pro-moieties” as described, for example, in “Design of Prodrugs” by H Bundgaard (Elseview, 1985).

### G. Methods of Treatment

The present invention further comprises methods for treating a condition in a subject having or susceptible to having such a condition, by administering to the subject a therapeutically-effective amount of one or more compounds of Formulae (I) through (IV) as described above. In one embodiment, the treatment is preventative treatment. In another embodiment, the treatment is palliative treatment. In another embodiment, the treatment is restorative treatment.

1. Conditions

The conditions that can be treated in accordance with the present invention include platelet aggregation mediated conditions such as atherosclerotic cardiovascular conditions, cerebrovascular conditions and peripheral arterial conditions, particularly those related to thrombosis. In another embodiment, platelet aggregation mediation conditions may be treated. In still another embodiment, the compounds of the present invention can be used to treat platelet dependent thrombosis or a platelet dependent thrombosis-related condition.

In one embodiment, the compounds of the invention can be used to treat acute coronary syndrome. Acute coronary syndrome includes, but is not limited to, angina (such as unstable angina) and myocardial infarction (such as non-ST-segment elevation myocardial infarction, non-Q-wave myocardial infarction and Q-wave myocardial infarction).

In another embodiment, the compounds of the present invention can be used to treat stroke (such as thrombotic stroke, ischemic stroke, embolic stroke and transient ischemic attack). In another embodiment, the compounds of the present invention can be used to treat a subject who has suffered from at least one event selected from the group consisting of myocardial infarction and stroke. In another embodiment, the compounds of the present invention can be used to treat atherosclerotic events selected from the group consisting of myocardial infarction, transient ischemic attack, stroke, and vascular death.

In another embodiment, the compounds of the present invention can be used to treat thrombotic and restenotic complications or treat reocclusion following invasive procedures including, but not limited to, angioplasty, percutaneous coronary intervention, carotid endarterectomy, coronary arterial bypass graft (“CABG”) surgery, vascular graft surgery, stent placements, lower limb arterial graft, prosthetic heart valve placement, hemodialysis and insertion of endovascular devices and prostheses.

In another embodiment, the compounds of the present invention can be used to treat platelet dependent thrombosis or a platelet dependent thrombosis-related condition that is selected from the group consisting of acute coronary syndrome; unstable angina; non Q-wave myocardial infarction; non-ST segment elevation myocardial infarction; acute myocardial infarction; deep vein thrombosis; pulmonary embolism; ischemic necrosis of tissue; atrial fibrillation; thrombotic stroke; embolic stroke; recent myocardial infarction; peripheral arterial disease; peripheral vascular disease; refractory ischemia; preeclampsia, eclampsia; acute ischemic stroke; disseminated intravascular coagulation; and thrombotic cytopenic purpura.

In another embodiment, the compounds of the present invention can be used to treat thrombotic or restenotic complications or reocclusion. In still another embodiment the thrombotic or restenotic complications or reocclusion are selected from the group consisting of angioplasty, percutaneous coronary intervention, carotid endarterectomy, post-coronary arterial bypass graft surgery, vascular graft surgery, stent placements, lower limb arterial graft, atrial fibrillation, prosthetic heart valve placement, hemodialysis and insertion of endovascular devices and prostheses.

In another embodiment, the compounds of the present invention can be used to reduce the risk in a subject of experiencing vascular events. In still another embodiment, the vascular events are selected from the group consisting of myocardial infarction, stable angina, coronary artery disease, ischemic stroke, transient ischemic attack and peripheral arterial disease.

In another embodiment, the compounds of the present invention can be used to treat hypertension.

In another embodiment, the compounds of the present invention can be used to treat angiogenesis.

2. Administration and Dosing

Typically, a compound described in this specification is administered in an amount effective to inhibit ADP mediated platelet aggregation. The compounds of the present invention are administered by any suitable route in the form of a pharmaceutical composition adapted to such a route, and in a dose effective for the treatment intended. Therapeutically effective doses of the compounds required to prevent or arrest the progress of or to treat the medical condition are readily ascertained by one of ordinary skill in the art using preclinical and clinical approaches familiar to the medicinal arts.

The compounds of the invention may be administered orally. Oral administration may involve swallowing, so that the compound enters the gastrointestinal tract, or buccal or sublingual administration may be employed by which the compound enters the blood stream directly from the mouth.

In another embodiment, the compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

In another embodiment, the compounds of the invention may also be administered topically to the skin or mucosa, that is, dermally or transdermally. In another embodiment, the compounds of the invention can also be administered intranasally or by inhalation. In another embodiment, the compounds of the invention may be administered rectally or vaginally. In another embodiment, the compounds of the invention may also be administered directly to the eye or ear.

The dosage regimen for the compounds and/or compositions containing the compounds is based on a variety of factors, including the type, age, weight, sex and medical condition of the patient; the severity of the condition; the route of administration; and the activity of the particular compound employed. Thus the dosage regimen may vary widely. Dosage levels of the order from about 0.01 mg to about 100 mg, per kilogram of body weight per day are useful in the treatment of the above-indicated conditions. In one embodiment, the total daily dose of a compound of Formulae (I) through (IV) (administered in single or divided doses) is typically from about 0.01 to about 100 mg/kg. In another embodiment, total daily dose of the compound of Formulae (I) through (IV) is from about 0.1 to about 50 mg/kg, and in another embodiment, from about 0.5 to about 30 mg/kg (i.e., mg compound of Formulae (I) through (IV) per kg body weight). In one embodiment, dosing is from 0.01 to 10 mg/kg/day. In another embodiment, dosing is from 0.1 to 1.0 mg/kg/day. Dosage unit compositions may contain such amounts or submultiples thereof to make up the daily dose. In many instances, the administration of the compound will be repeated a plurality of times in a day (typically no greater than 4 times). Multiple doses per day typically may be used to increase the total daily dose, if desired.

For oral administration, the compositions may be provided in the form of tablets containing 0.01, 0.05, 0.1, 0.5, 1.0, 2.5, 5.0, 10.0, 15.0, 25.0, 50.0, 75.0, 100, 125, 150, 175, 200, 250 and 500 milligrams of the active ingredient for the symptomatic adjustment of the dosage to the patient to be treated. A medicament typically contains from about 0.01 mg to about 500 mg of the active ingredient, or in another embodiment, from about 1 mg to about 100 mg of active ingredient. Intravenously, doses may range from about 0.1 to about 10 mg/kg/minute during a constant rate infusion.

Suitable subjects to be treated according to the present invention include mammalian subjects. Mammals according to the present invention include, but are not limited to, canine, feline, bovine, caprine, equine, ovine, porcine, rodents, lagomorphs, primates, and the like, and encompass mammals in utero. In one embodiment, humans are suitable subjects. Human subjects may be of either gender and at any stage of development.

### H. Use in the Preparation of a Medicament

In one embodiment, the present invention comprises methods for the preparation of a pharmaceutical composition (or “medicament) comprising the compounds of Formulae (I) through (IV) in combination with one or more pharmaceutically-acceptable carriers and/or other active ingredients for use in treating a platelet aggregation mediated condition.

In another embodiment, the invention comprises the use of one or more compounds of Formulae (I) through (IV) in the preparation of a medicament for the treatment of acute coronary syndrome. In another embodiment, the invention comprises the use of one or more compounds of Formulae (I) through (IV) in the preparation of a medicament for the reduction of atherosclerotic events. In another embodiment, the invention comprises the use of one or more compounds of Formulae (I) through (IV) in the preparation of a medicament for the treatment of thrombosis. In another embodiment, the invention comprises the use of one or more compounds of Formulae (I) through (IV) in the preparation of a medicament to be co-administered before, during or after revascularization procedures, including, but not limited to, lower limb arterial graft, carotid endarterectomy, coronary artery bypass surgery, atrial fibrillation, prosthetic heart valve placement, hemodialysis and placement of mechanical devices.

### I. Pharmaceutical Compositions

For the treatment of the conditions referred to above, the compounds of Formulae (I) through (IV) can be administered as compound per se. Alternatively, pharmaceutically acceptable salts are suitable for medical applications because of their greater aqueous solubility relative to the parent compound.

In another embodiment, the present invention comprises pharmaceutical compositions. Such pharmaceutical compositions comprise compounds of Formulae (I) through (IV) presented with a pharmaceutically-acceptable carrier. The carrier can be a solid, a liquid, or both, and may be formulated with the compound as a unit-dose composition, for example, a tablet, which can contain from 0.05% to 95% by weight of the active compounds. Compounds of Formulae (I) through (IV) may be coupled with suitable polymers as targetable drug carriers. Other pharmacologically active substances can also be present.

The active compounds of the present invention may be administered by any suitable route, wherein the compound may comprise forms of a pharmaceutical composition adapted to such a route, and in a dose effective for the treatment intended. The active compounds and compositions, for example, may be administered orally, rectally, parenterally, or topically. Oral administration of a solid dose form may be, for example, presented in discrete units, such as hard or soft capsules, pills, cachets, lozenges, or tablets, each containing a predetermined amount of at least one compound of the present invention. In another embodiment, the oral administration may be in a powder or granule form. In another embodiment, the oral dose form is sub-lingual, such as, for example, a lozenge. In such solid dosage forms, the compounds of Formulae (I) through (IV) are ordinarily combined with one or more adjuvants. Such capsules or tablets may contain a controlled-release formulation. In the case of capsules, tablets, and pills, the dosage forms also may comprise buffering agents or may be prepared with enteric coatings. In another embodiment, oral administration may be in a liquid dose form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as wetting, emulsifying, suspending, flavoring (e.g., sweetening), and/or perfuming agents.

In another embodiment, the present invention comprises a parenteral dose form. “Parenteral administration” includes, for example, subcutaneous injections, intravenous injections, intraperitoneally, intramuscular injections, intrasternal injections, and infusion. Injectable preparations (e.g., sterile injectable aqueous or oleaginous suspensions) may be formulated according to the known art using suitable dispersing, wetting agents, and/or suspending agents. In another embodiment, the present invention comprises a topical dose form. “Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

In another embodiment, the compounds of the present invention may be administered in combination with treatment of restenosis resulting from angioplasty, including, without limitation, such therapies as inserting a stent at the site of angioplasty. The stent itself comprises the compound of the present invention and is used as a carrier to effect local delivery of the compound to the target vessel. The compound is coated on, adsorbed on, affixed to or present on the surface of the stent or is otherwise present in or on the matrix of the stent, either alone or in combination with other active drugs and pharmaceutically acceptable carriers, adjuvants, binding agents and the like.

One exemplary stent comprises a compound of the invention in the form of an extended release composition that provides for release of the compound over an extended period of time. Another exemplary stent comprises a hydrogel containing entrapped the compound, wherein the hydrogel is attached directly onto a stent or attached to a polymer coated stent. This hydrogel, containing entrapped the compound of this invention, can be used as a topcoat on a stent to provide a fast release, bolus-like localized administration of the entrapped compound. Under the hydrogel/therapeutic agent topcoating, other biodegradable polymer coatings (e.g., poly ester-amide with covalently conjugated or matrixed drugs) can be positioned to create a sustained release local drug/biologic delivery system. This hydrogel system is exemplified in U.S. Pat. No. 6,716,445 (granted Apr. 6, 2004).

Formulations suitable for topical administration to the eye include, for example, eye drops wherein the compound of this invention is dissolved or suspended in suitable carrier. A typical formulation suitable for ocular or aural administration may be in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (including, but not limited to, an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the present invention comprises a rectal dose form. Such rectal dose form may be in the form of, for example, a suppository. Cocoa butter is a traditional suppository base, but various alternatives may be used as appropriate.

Other carrier materials and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman, et al., Eds., Pharmaceutical Dosage Forms, Marcel Decker, New York, N.Y., 1980; and Kibbe, et al., Eds., Handbook of Pharmaceutical Excipients (3^{rd} Ed.), American Pharmaceutical Association, Washington, 1999.

### J. Co-Administration

The compounds of the present invention can be used, alone or in combination with other therapeutic agents, in the treatment of various conditions or disease states. The compound(s) of the present invention and other therapeutic agent(s) may be may be administered simultaneously (either in the same dosage form or in separate dosage forms) or sequentially.

The administration of two or more compounds “in combination” means that the two compounds are administered closely enough in time that the presence of one alters the biological effects of the other. The two or more compounds may be administered simultaneously, concurrently or sequentially. Additionally, simultaneous administration may be carried out by mixing the compounds prior to administration or by administering the compounds at the same point in time but at different anatomic sites or using different routes of administration.

The phrases “concurrent administration,” “co-administration,” “simultaneous administration,” and “administered simultaneously” mean that the compounds are administered in combination. In one embodiment, compounds of Formulae (I) through (IV) may be co-administered with an oral antiplatelet agent, including, but not limited to, aspirin, dipyridamole, cilostazol and anegrilide hydrochloride. In still another embodiment, compounds of Formulae (I) through (IV) may be co-administered with aspirin.

In another embodiment, compounds of Formulae (I) through (IV) may be co-administered with a glycoprotein IIIb/IIIa inhibitor, including, but not limited to, abciximab, eptifibatide and tirofiban. In still another embodiment, compounds of Formulae (I) through (IV) may be co-administered with eptifibatide.

In another embodiment, compounds of Formulae (I) through (IV) may be co-administered with a heparin or heparinoid, including, but not limited to, heparin sodium, enoxaparin sodium, dalteparin sodium, ardeparin sodium, nadroparin calcium, reviparin sodium, tinzaparin sodium and fondaparinux sodium.

In another embodiment, compounds of Formulae (I) through (IV) may be co-administered with a direct thrombin inhibitor, including, but not limited to, danaparoid, hirudin, bivalirudin and lepirudin.

In another embodiment, compounds of Formulae (I) through (IV) may be co-administered with an anti-coagulant including, but not limited to, warfarin, warfarin sodium, 4-hydroxycoumarin, dicoumarol, phenprocoumon, anisindione, acenocoumerol and phenindione. In still another embodiment, compounds of Formulae (I) through (IV) may be co-administered with warfarin sodium.

In another embodiment, compounds of Formulae (I) through (IV) may be co-administered with an oral factor Xa inhibitor including, but not limited to, ximelagatran, melagatran, dabigatran etexilate and argatroban. In still another embodiment, compounds of Formulae (I) through (IV) may be co-administered with ximelagatran.

In another embodiment, compounds of Formulae (I) through (IV) may be co-administered with a fibrinolytic including, but not limited to, streptokinase, urokinase, tissue plasminogen activator, tenecteplase, reteplase, alteplase and aminocaproic acid.

In another embodiment, compounds of Formulae (I) through (IV) may be co-administered with an investigational compound useful in treating platelet aggregation including, but not limited to, BAY 59-7939, YM-60828, M-55532, M-55190, JTV-803 and DX-9065a.

### K. Kits

The present invention further comprises kits that are suitable for use in performing the methods of treatment or prevention described above. In one embodiment, the kit contains a first dosage form comprising one or more of the compounds of the present invention and a container for the dosage, in quantities sufficient to carry out the methods of the present invention.

In another embodiment, the kit of the present invention comprises one or more compounds of Formulae (I) through (IV) and an oral antiplatelet agent, including, but not limited to, aspirin, dipyridamole, cilostazol and anegrilide hydrochloride. In still another embodiment, the kit of the present invention comprises one or more compounds of Formulae (I) through (IV) and aspirin. In another embodiment, the kit of the present invention comprises one or more compounds of Formulae (I) through (IV) and a glycoprotein IIIb/IIIa inhibitor, including, but not limited to, abciximab, eptifibatide and tirofiban. In still another embodiment, the kit of the present invention comprises one or more compounds of Formulae (I) through (IV) and eptifibatide.

In another embodiment, the kit of the present invention comprises one or more compounds of Formulae (I) through (IV) and a heparin or heparinoid, including, but not limited to, heparin sodium, enoxaparin sodium, dalteparin sodium, ardeparin sodium, nadroparin calcium, reviparin sodium, tinzaparin sodium and fondaparinux sodium.

In another embodiment, the kit of the present invention comprises one or more compounds of Formulae (I) through (IV) and a direct thrombin inhibitor, including, but not limited to, danaparoid, hirudin, bivalirudin and lepirudin.

In another embodiment, the kit of the present invention comprises one or more compounds of Formulae (I) through (IV) and an anti-coagulant including, but not limited to, warfarin, warfarin sodium, 4-hydroxycoumarin, dicoumarol, phenprocoumon, anisindione, acenocoumerol and phenindione. In still another embodiment, the kit of the present invention comprises one or more compounds of Formulae (I) through (IV) and warfarin sodium.

In another embodiment, the kit of the present invention comprises one or more compounds of Formulae (I) through (IV) and an oral factor Xa inhibitor including, but not limited to, ximelagatran, melagatran, dabigatran etexilate and argatroban. In still another embodiment, the kit of the present invention comprises one or more compounds of Formulae (I) through (IV) and ximelagatran.

In another embodiment, the kit of the present invention comprises one or more compounds of Formulae (I) through (IV) and a fibrinolytic including, but not limited to, streptokinase, urokinase, tissue plasminogen activator, tenecteplase, reteplase, alteplase and aminocaproic acid. In another embodiment, the kit of the present invention comprises one or more compounds of Formulae (I) through (IV) and an investigational compound useful in treating platelet aggregation including, but not limited to, BAY 59-7939, YM-60828, M-55532, M-55190, JTV-803 and DX-9065a.

### L. Intermediates

In another embodiment, the invention relates to the intermediates described in Working Examples 23, 34 and 35, which are useful for preparing the thieno[2,3-d]pyrimidine compounds of Formulae (I)-(IV).

### M. General Synthetic Schemes

The starting materials used herein are commercially available or may prepared by routine methods known in the art (such as those methods disclosed in standard reference books such as the COMPENDIUM OF ORGANIC SYNTHETIC METHODS, Vol. I-VI (published by Wiley-Interscience)). The compounds of the present invention may be prepared using the methods illustrated in the general synthetic schemes and experimental procedures detailed below. The general synthetic schemes are presented for purposes of illustration and are not intended to be limiting.

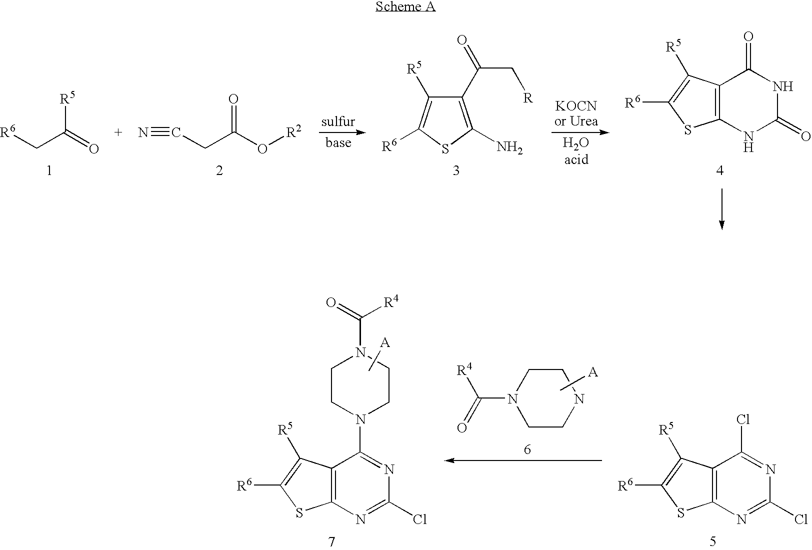

Scheme A. Thienopyrimidines may be prepared by various methods. One method for the preparation of thienopyrimidine 7 is depicted in Scheme A. Commercially available aldehyde/ketone 1 and esters 2 are combined in the presence of sulfur to give thiophene 3 using the general method of Tinney et al. (J. Med. Chem. (1981) 24, 878-882). Thiophene 3 is then treated with potassium cyanate or urea in the presence of water and an acid such as acetic,acid to give dione 4. Dione 4 is then treated with a chloride source such as phosphorous oxychloride, thionyl chloride, or phosphorous pentachloride with or without the presence of a tertiary amine or concentrated HCl and with or without added inert solvent such as dimethylformamide at temperatures ranging from 75° C. to 175° C., optionally with an excess of phosphorous oxychloride in a sealed vessel at 130-175° C., to give dichloropyrimidine 5. Dichloropyrimidine 5 is then treated with piperazine 6 (see Scheme B) in the presence of a base such as trialkylamine, pyridine, potassium carbonate, sodium carbonate, cesium carbonate, and other bases well known to those versed in the art and in the presence of a solvent such as THF, acetonitrile, dichloromethane, dialkyl ether, toluene, DMF, N-methyl pyrrolidinone and the like at temperatures ranging from room temperature to the reflux temperature of the solvent to give thienopyrimidine 7.

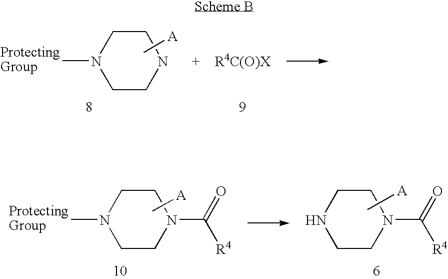

Scheme B. Scheme B depicts the preparation of intermediate 6. Protected piperazine 8 is commercially available or can be prepared by (1) attaching a suitable protecting group including, but not limited to, Boc, Cbz, Fmoc and benzyl, to one of the nitrogen ring atoms of the piperazine and (2) reacting with alkylOCOCl or (alkylOCO)₂O). Protected piperazine 8 is then acylated using acyl reagent 9, where acyl reagent 9 is used in its acid form (X═OH) in the presence of a coupling agent. Suitable coupling agents include, but are not limited to, DCC, EDC, DEPC, HATU, HBTU and CDI. In an alternative preparation of intermediate 6, acyl reagent 9 is used in the form of an acid halide (X═Cl, Br, F) or anhydride (X═O(COR₄))in the presence of a base, including, but not limited to, a trialkylamine, pyridine, or an alkaline earth metal carbonate and in the presence of inert solvents such as THF, dichloromethane, acetonitrile, toluene, dialkyl ether, DMF, N-methylpyrrolidinone, dimethylacetamide and the like at temperatures ranging between ice/water temperature to the reflux temperature of the solvent, to give bisamide 10. Bisamide 10 is converted to piperazine 6 using methods well know to those versed in the art, many of which are discussed by Greene and Wuts in Protective Groups in Organic Synthesis, Third Ed., Wiley-lnterscience, pp. 502-550. When the protecting group of bisamide 10 is a benzyl group, then removal of the benzyl group to give intermediate 6 is accomplished using standard methods known in the art (e.g., those discussed by Greene and Wuts in Protective Groups in Organic Synthesis, Third Ed., Wiley-lnterscience, pp. 502-550).

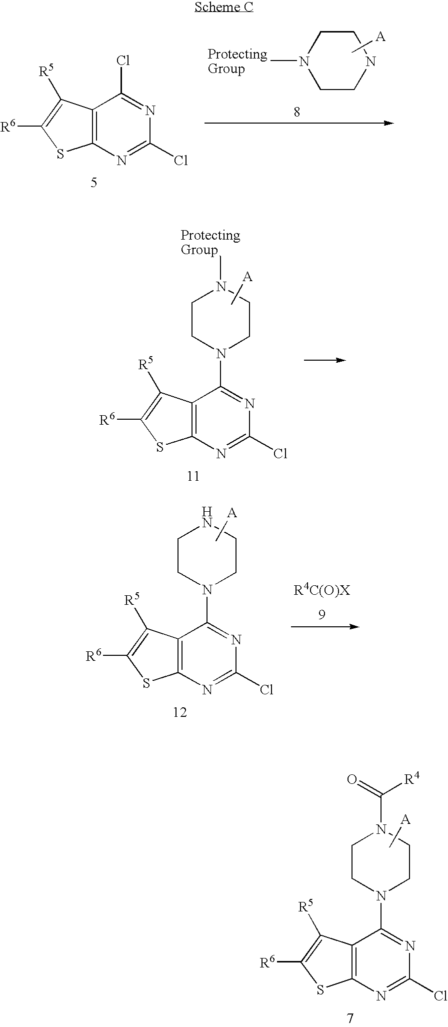

Scheme C. The order of addition of various functionalities to the thienopyrimidine can be changed to take advantage of commercially available materials or in order to avoid reactivities at other parts of the molecule. An alternative method for the preparation of thienopyrmidine 7 using an order of addition differing from that of Scheme A is shown in Scheme C. Dichloropyrimidine 5 (Scheme A) is aminated with 8 (Scheme B) in inert solvents at temperatures ranging from room temperature to the boiling point of the solvent to give pyrimidine 11. The amination may be done using excess 8 or in the presence of a base, including but not limited to, a trialkylamine, pyridine, or an alkaline earth metal carbonate. Removal of the protecting group to give pyrimidine-piperazine 12 is achieved using standard deprotection method, such as those discussed by Greene and Wuts in Protective Groups in Organic Synthesis, Third Ed., Wiley-lnterscience, pp. 502-550. Thienopyrimidine 7 is obtained upon combining acyl reagent 9 (X═OH) with pyrimidine-piperazine 11 using coupling reagents, many of which are well known to those versed in the art and include but are not limited to DCC, EDC, DEPC, HATU, HBTU and CDI. Alternatively, 9 is used in the form of,an acid halide X═Cl, Br, F) or anhydride (X═O(COR₄)) in the presence of a base, wherein an exemplary base is trialkylamine, pyridine, or an alkaline earth metal carbonate and in the presence of inert solvents including, but not limited to, THF, dichloromethane, acetonitrile, toluene, dialkyl ether, DMF, N-methylpyrrolidinone and the like at temperatures ranging between ice/water temperature to the reflux temperature of the solvent.

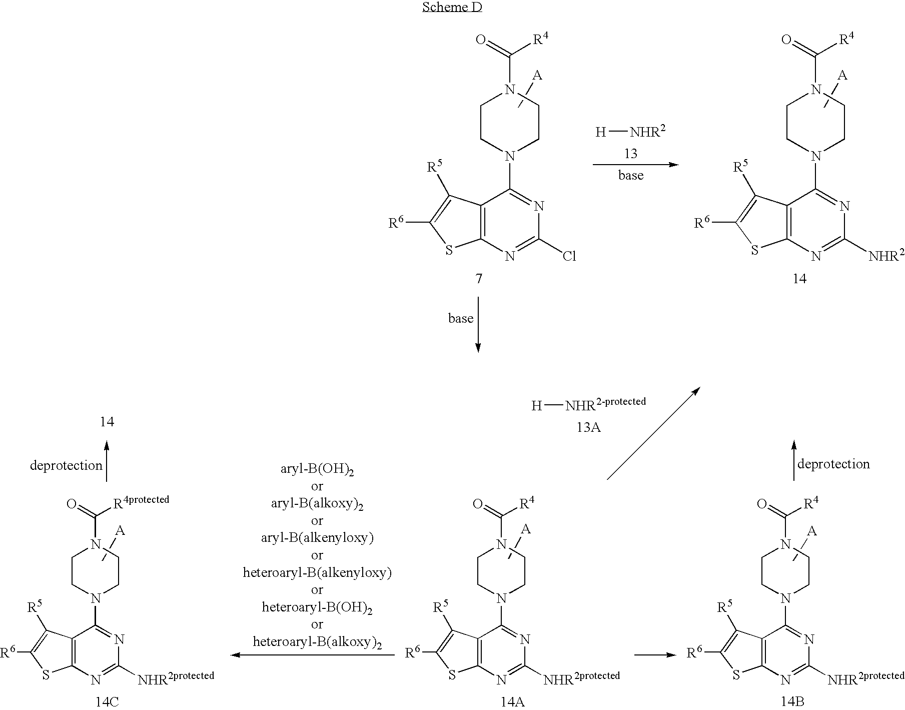

Scheme D. Elaboration of thienopyrimidine 7 to substituted thienopyrimidine 14 is accomplished by treating thienopyrimidine 7 with H-NHR² (13), and where H-NHR² is commercially available or may be prepared by methods well-known to those versed in the art.

Reagent 13 is combined with thienopyrimidine 7 in the presence of a base and an inert solvent. Reagent 13 may be used in a one- to ten-fold excess, wherein an exemplary base is a trialkylamine base, an exemplary solvent is N-methyl pyrrolidinone or butanol, and the temperature is between room temperature and 160° C. The chemist may choose to omit added base and instead use excess HNHR₇ as the baseTo reduce undesired reactions, reagent 13 can be protected first (i.e. R² is in a protected form) namely reagent 13A, to give substituted thienopyrimidine 14A, wherein the protecting group may be removed at a later stage to give substituted thienopyrimidine 14. Reagent 13A is commercially available or may be prepared by methods well known to those versed in the art. For example, when R₇ is desired to be an alkyl diol, the diol of H-NHR² may be protected using methods known in the art. Methods for the synthesis and removal diol protecting groups are discussed by Greene and Wuts in “Protective Groups in Organic Synthesis,” Third Ed., Wiley-lnterscience, pp. 201-245.

Alternatively, R²in 14A may be an alkyl aldehyde or alkyl ketone in its protected form. Many protected aldehydes and ketones 1 3A are commercially available. Conventional procedures for the synthesis and removal of aldehyde and ketone protecting groups are known in the art (e.g. the procedures discussed by Greene and Wuts in “Protective Groups in Organic Synthesis,” Third Ed., Wiley-Interscience, pp. 201-245.) After removal of the aldehyde or ketone protecting group to give substituted thienopyrimidine 14B, the aldehyde or ketone may be further manipulated. For example, treatment of an aldehyde with an oxidizing agent such as 3-chloroperoxbenzoic acid and the like gives substituted thienopyrimidine 14 where R² contains a carboxylic acid. Treatment of an aldehyde or ketone with an amine in the presence of a reducing agent such as sodium cyanoborohydride, sodium triacetoxyborohydride, tri(trifluoroacetoxy)borohydride, or hydrogen gas and a metal catalyst give substituted thienopyrimidine 14 where R² contains an amino group.

When R₄ is phenyl or heteroaryl substituted with Br, I, Cl, and O-triflate, then additional manipulations of R⁴ may be carried out using standard methods known in the art. For example, aryl- or heteroaryl-boronic acids or esters, many of which are commercially available, may be reacted, in the presence of a metal catalyst, with substituted thienopyrimidine 14A to give biaryl substituted thienopyrimidine 14C. Thus, treatment with an aryl or heteroaryl boronic acid or heteroaryl or aryl boronic acid ester such as [(aryl or heteroaryl)-B(OH)_{2]} or [(aryl or heteroaryl)-B(OR^{a})(OR^{b}) (where R^{a} and R^{b} are each C₁-C₆ alkyl, or when taken together, R^{a} and R^{b} are C₂-C₁₂ alkylene)] in the presence of a metal catalyst with or without a base in an inert solvent yields biaryl substituted thienopyrimidine 14C. Metal catalysts in these transformations include, but are not limited to, salts or phosphine complexes of Cu, Pd, or Ni (for example, Cu(OAc)₂, PdCl₂(PPh₃)₂, NiCl₂(PPh₃)₂). Bases may include, but are not limited to, alkaline earth metal carbonates, alkaline earth metal bicarbonates, alkaline earth metal hydroxides, alkali metal carbonates, alkali metal bicarbonates, alkali metal hydroxides, alkali metal hydrides, alkali metal alkoxides, alkaline earth metal hydrides, alkali metal dialkylamides, alkali metal bis(trialkylsilyl)amides, trialkyl amines or aromatic amines.

In one embodiment, the alkali metal hydride is sodium hydride. In another embodiment, the alkali metal alkoxide is sodium methoxide. In another embodiment, the alkali metal alkoxide is sodium ethoxide. In another embodiment, the alkali metal dialkylamide is lithium diisopropylamide. In another embodiment, the alkali metal bis(trialkylsilyl)amide is sodium bis(trimethylsilyl)amide. In another embodiment, the trialkyl amine is diisopropylethylamine. In another embodiment, the trialkylamine is triethylamine. In another embodiment, the aromatic amine is pyridine.

Inert solvents may include, but are not limited to, acetonitrile, dialkyl ethers, cyclic ethers, N,N-dialkylacetamides (dimethylacetamide), N,N-dialkylformamides, dialkylsulfoxides, aromatic hydrocarbons or haloalkanes.

In one embodiment, the dialkyl ether is diethyl ether. In another embodiment, the cyclic ether is tetrahydrofuran. In another embodiment, the cyclic ether is 1,4-dioxane. In another embodiment the N,N-dialkylacetamide is dimethylacetamide. In another embodiment, the N,N-dialkylformamide is dimethylformamide. In another embodiment, the dialkylsulfoxide is dimethylsulfoxide. In another embodiment, the aromatic hydrocarbon is benzene. In another embodiment, the aromatic hydrocarbon is toluene. In another embodiment, the haloalkane is methylene chloride.

Exemplary reaction temperatures range from room temperature up to the boiling point of the solvent employed. Non-commercially available boronic acids or boronic acid esters may be obtained from the corresponding optionally substituted aryl halide as described in Tetrahedron, 50, 979-988 (1994). Alternatively, as described in Tetrahedron, 50, 979-988 (1994), one may convert the R⁴ substitueni to tne corresponding boronic acid or boronic acid ester (OH)₂B— or (OR^{a})(OR^{b})B— and obtain the same products set forth above by treating with a suitable aryl or heteroaryl halide or.triflate. The protecting group on R′² of 14C is then removed using conditions discussed above to give 14.

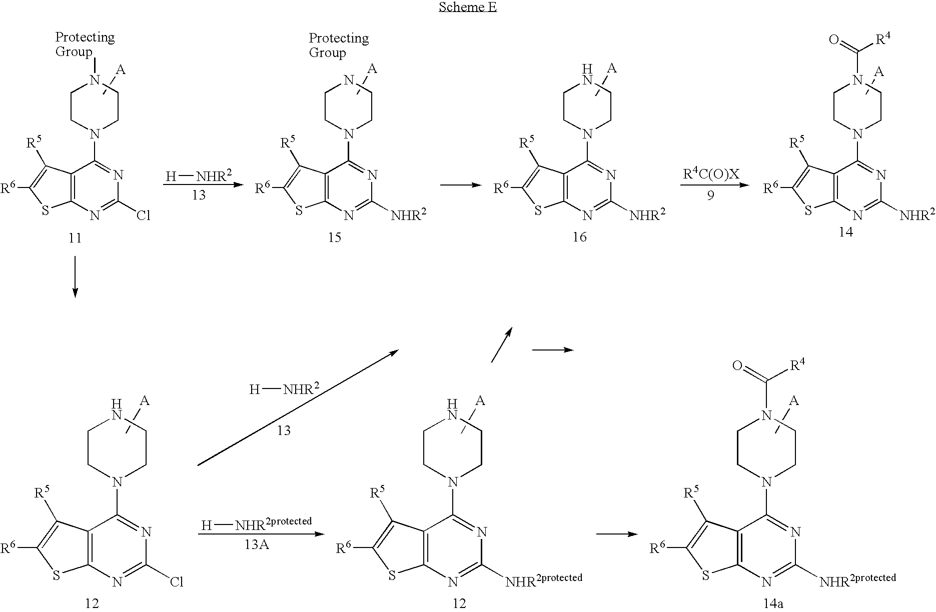

Scheme E. The order of addition of various functionalities of the thienopyrimidine can be changed in the preparation of substituted thienopyrimidine 14 in order to take advantage of commercially available materials or in order to avoid reactivities at other parts of the molecule. Another method for the preparation of substituted thienopyrimidine 14 is shown in Scheme E, where piperazinyl pyrimidine 11 is combined with reagent 13 where H-NHR₇ is commercially available or may be prepared by methods well-known to those versed in the art, to give di-substituted thienopyrimidine 15. Reagent 13 is combined with piperazinyl pyrimidine 11 in the presence of a base and an inert solvent to give di-substituted thienopyrimidine 15. Reagent 13 may be used in a one- to ten-fold excess, wherein an exemplary base is a trialkylamine base, an exemplary solvent is N-methylpyrrolidinone or butanol, and the temperature is between room temperature and 160° C. The chemist may choose to omit added base and instead use excess HYR₇ (13) as the base. Disubstituted thienopyrmidine 15 is then combined with a reagent suitable for the removal of the protecting group to give amine 16. Suitable means for removal of the the protecting group depends on the nature of the group. For example, to remove the protecting group, BOC, one may dissolve disubstituted thienopyrimidine in a trifluoroacetic acid/dichloromethane mixture. A second exemplary method is the addition of hydrogen chloride gas dissolved in an alcohol or ether such as methanol or dioxane. When complete, the solvents are removed under reduced pressure to give the corresponding amine as the corresponding salt, i.e. trifluoroacetic acid or hydrogen chloride salt. However, if desired, the amine can be purified further by means well known to those skilled in the art, such as for example, recrystallization. Further, if the non-salt form is desired that also can be obtained by means known to those skilled in the art, such as for example, preparing the free base amine via treatment of the salt with mild basic conditions.

Additional deprotection conditions and deprotection conditions for other protecting groups can be found in T. W. Green and P. G. M. Wuts in “Protective Groups in Organic Chemistry,” John Wiley and Sons, 1999, pp. 502-550. Thienopyrimidine 14 is obtained upon combining acyl reagent 9 (X═OH) with amine 16 using coupling reagents, which include but are not limited to DCC, EDC, DEPC, HATU, HBTU, CDI, or 9 is used in the form of an acid halide (X═Cl, Br, F) or anhydride (X═O(COR₄)) in the presence of a base, wherein an exemplary base is a trialkylamine, pyridine, or an alkaline earth metal carbonate and in the presence of inert solvents such as THF, dichloromethane, acetonitrile, toluene, dialkyl ether, DMF, N-methylpyrrolidinone and the like at temperatures ranging between ice/water temperature to the reflux temperature of the solvent. Depending upon the nature of the various substituents, it may be desirable to change the order of addition of the substituents. For example, the protecting group of 11 may be removed to give 12 as described in Scheme C. Pyrimidine piperazine 12 may then be reacted with 13 in the same manner as described for the conversion of 7 to 14 in Scheme D to give 16. Alternatively, pyrimidine piperazine 12 may be reacted with a protected form of 13, namely 13A, to give 17. Addition of R⁴COX (9) to 17 gives 14A, which then may be further manipulated as described for Scheme D. Alternatively, amine 17 may be converted to 16 by methods described for the conversion of 14A to 14 in Scheme D.

### N. Working Examples

The following illustrate the synthesis of various compounds of the present invention. Additional compounds within the scope of this invention may be prepared using the methods illustrated in these Examples, either alone or in combination with techniques generally known in the art.

### EXAMPLE 1

### Methyl 2-amino-5-ethylthiophene-3-carboxylate

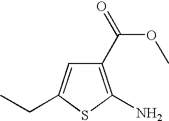

To a mixture of sulfur (6.4 g) in DMF (25 mL) was added methyl cyanoacetate (19.8 g) and triethylamine (15 mL,) under nitrogen. The mixture was stirred for 10 minutes at which time butyraldehyde (18 mL) was added drop-wise at a sufficient rate to maintain a temperature of 50° C. The mixture was then stirred at room temperature for 20 hours. The mixture was partitioned between brine and ethyl acetate. The layers were separated and the organic layer washed three times with brine, dried over anhydrous magnesium sulfate and concentrated. The residue was chromatographed on silica gel using ethyl acetate-hexanes (10/90) to give a solid. The solid was slurried in hexanes, collected, and dried to give 25.74 g of the desired product as an off-white solid: MS (ESI+) for C8 H11 N1 O2 S₁ m/z 186.0598 (M+H)⁺. ¹H NMR (300 MHz, CDCl₃) δ 1.22 (t, 3 H), 2.6 (q, 2 H), 3.79 (s, 3 H), 5.79 (s, 2 H), 6.62 (s, 1 H).

### EXAMPLE 2

### 6-Ethyl-4a,7a-dihydrothieno[2,3-d]pyrimidine-2,4-diol

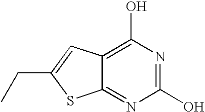

To a mixture of methyl 2-amino-5-ethylthiophene-3-carboxylate (EXA 1; 25.2 g) in glacial acetic acid (450 mL) and water (45 mL) was added drop-wise a solution of potassium cyanate (30.9 g) in water (150 mL). The mixture exothermed to 33° C. and some gas was evolved. A white precipitate formed during addition. The mixture was stirred at room temperature for 20 hours. Ice water (300 mL) was added to the mixture and the solids were collected by filtration and washed with water (200 mL). The solids were transferred to a round-bottomed flask to which was added 6% aq. sodium hydroxide (500 mL). The mixture was refluxed for 2 h, then cooled to room temperature. The temperature was further lowered to 5° C. in an ice-water bath. The pH was adjusted to ˜6 with concentrated hydrochloric acid. The resulting solids were collected, washed with water, and dried under reduced pressure to give 16.39 g of the title compound as an off-white solid. The material was subsequently azeotroped using THF/toluene to remove any residual water: MS (ESI+) for C8 H8 N2 O2 S₁ m/z 197.0 (M+H)⁺; ¹H NMR 400 MHz, DMSO-d₆) δ 1.24 (t, 3 H), 2.74 (q, 2 H), 6.85 (s, 1 H), 11.1 (s, 1 H), 11.8 (s, 1 H).

### EXAMPLE 3

### 2,4-Dichloro-6-ethylthieno[2,3-d]pyrimidine

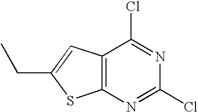

6-Ethyl-4a,7a-dihydrothieno[2,3-d]pyrimidine-2,4-diol (EXAM 2; 4.0 g,) was placed into a glass pressure vessel with phosphorus oxychloride (35 mL). The mixture was heated to 150° C. for 1.5 hours. The mixture was cooled to room temperature and concentrated under reduced pressure. Residual phosphorus oxychloride was azeotroped twice with toluene (50 mL) under reduced pressure. The residue was partitioned between saturated sodium bicarbonate and dichloromethane. The resulting layers were separated and decolorizing carbon (1 g) was added to the organic layer. The organic layer was filtered through anhydrous magnesium sulfate and the filtrate was concentrated to dryness under reduced pressure to give 3.96 g of the title compound: MS (ESI+) for C8 H6 Cl2 N2 S₁ m/z 233.0 (M+H)⁺; ¹H NMR (300 MHz, CDCl₃) δ 1.4 (t, 3 H), 3.0 (q, 2 H), 7.1 (s, 1 H).

### EXAMPLE 4

### Tert-butyl 4-(phenylacetyl)piperazine-1-carboxylate

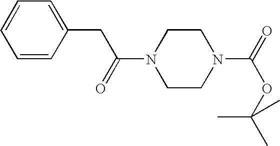

To a mixture of Boc-piperazine (4.2 g) in dry THF (30 mL) in a round bottomed flask in an ice-water bath was added triethylamine (3.14 mL). Phenyl acetyl chloride (2.9 mL) was added drop-wise such that the temperature remained below 15° C. Once addition was complete, the mixture was removed from the ice bath and allowed to stir at room temperature for 2 hours. The solvents were removed under reduced pressure and the residue partitioned between brine and ethyl acetate. The layers were separated and the organic layer washed with brine. The organic layer was then dried over anhydrous magnesium sulfate and concentrated. Hexanes were added and the resulting solids were collected via filtration to give 6.24 g of the title compound: MS (ESI+) for C17 H24 N2 O3 m/z 327.0 (M+H+Na)⁺; ¹H NMR (300 MHz, CDCl₃) δ 1.44 (s, 9 H), 3.2 (m, 2 H), 3.4 (m, 4 H), 3.6 (m, 2 H), 7.25 (m, 3 H), 7.33 (m, 2 H).

### EXAMPLE 5

### 1-(Phenylacetyl)piperazine

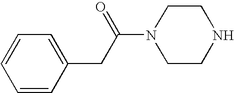

To a mixture of tert-butyl 4-(phenylacetyl)piperazine-1-carboxylate (EXAM 4; 6.0 g) in dichloromethane (5 mL) was added trifluoroacetic acid (5.0 mL). The mixture was stirred at room temperature for 8 hours. The solvents were removed under reduced pressure and the residue partitioned between saturated sodium bicarbonate and dichloromethane. The layers were separated and the aqueous layer extracted with dichloromethane. The combined dichloromethane extracts were dried using anhydrous magnesium sulfate and concentrated. The residue was chromatographed on silica gel using methanol-dichloromethane (8/92) with 0.1% ammonium hydroxide to give 2.01 g of the title compound: ¹H NMR (300 MHz, CDCl₃) δ 1.75 (s, 1 H), 2.66 (t, 2 H), 2.8 (t, 2 H), 3.4 (t, 2 H), 3.6 (t, 2 H), 3.7 (s, 2 H), 7.2 (m, 3 H), 7.3 (m, 2 H).

### EXAMPLE 6

### 2-Chloro-6-ethyl-4-[4-(phenylacetyl)piperazin-1-yl]thieno[2,3-d]pyrimidine

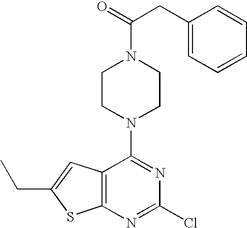

To a mixture of 2,4-dichloro-6-ethylthieno[2,3-d]pyrimidine (EXAM 3; 1.53 g) in dry THF (60 mL) was added diisopropylethylamine (4.6 mL) and 1-(phenylacetyl)piperazine (EXAM 5; 1.35 g). The mixture was stirred at room temperature for 2.5 h, at which time the mixture was partitioned between brine and ethyl acetate. The layers were separated and the organic layer washed with brine, dried over anhydrous magnesium sulfate and concentrated. The residue was chromatographed on silica gel using methanol-dichloromethane (2/98) to give 2.28 g of the title compound: MS (ESI+) for C20 H21 Cl1 N4 O1 S₁ m/z 401.0 (M+H)⁺; ¹H NMR (300 MHz, CDCl₃) δ 1.35 (t, 3 H), 2.85 (q, 2 H), 3.63 (m, 2 H), 3.74 (m, 2 H), 3.80 (s, 2 H), 3.85 (m, 2 H), 3.89 (m, 2 H), 6.9 (s, 1 H), 7.27 (m, 3 H), 7.34 (m, 2 H).

### EXAMPLE 7

### 6-Ethyl-4-[4-(phenylacetyl)piperazin-1-yl]-N-(3-phenylpropyl)thieno[2,3-d]pyrimidin-2-amine

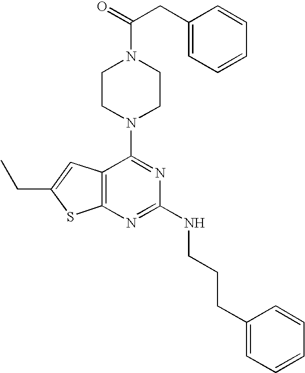

To 2-chloro-6-ethyl-4-[4-(phenylacetyl)piperazin-1-yl]thieno[2,3-d]pyrimidine (EXA 6; 0.15 g) was added 1-phenyl-3-propylamine (0.107 g). The mixture was heated at 125° C. for 18 hours. An additional 0.05 g of 1-phenyl-3-propylamine was added and the mixture was heated an additional 6 hours. The mixture was then cooled to room temperature. The residue was chromatographed on silica gel using methanol-chloroform (2/98) as eluent to give 0.061 g of the title compound: MS (ESI+) for C29 H33 N5 O1 S₁ m/z 500.2 (M+H)⁺; ¹H NMR (300 MHz, CDCl₃) δ 1.3 (t, 3 H), 1.9 (m, 2 H), 2.73 (t, 2 H), 2.8 (q, 2 H), 3.44 (m, 2 H), 3.57 (s, 4 H), 3.73-3.79 (m, 7 H), 4.82 (m, 1 H), 6.7 (s, 1 H), 7.25 (m, 3 H), 7.30 (m, 5 H), 7.37 (m, 2 H).

### EXAMPLE 8

### 6-Ethyl-N-(2-furylmethyl)-4-[4-(phenylacetyl)piperazin-1-yl]thieno[2,3-d]pyrimidin-2-amine

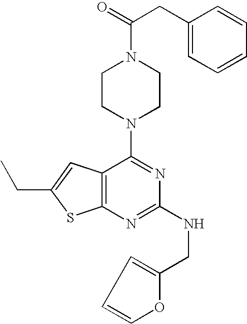

2-Chloro-6-ethyl-4-[4-(phenylacetyl)piperazin-1-yl]thieno[2,3-d]pyrimidine (EXA 6; 0.15 g) and furfurylamine (0.109 g) were placed into a 2-dram screw-capped vial and heated at 125° C. overnight. The mixture was then cooled to room temperature and the residue was chromatographed on silica gel using MeOH-dichloromethane (2/98) as eluent to give 0.0165 g of the title compound: MS (ESI+) for C25 H27 N5 O2 S₁ m/z 462.2 (M+H)⁺; ¹H NMR (300 MHz, CDCl₃) δ 1.26 (t, 3 H), 2.8 (q, 2 H), 3.5 (s, 1 H), 3.6 (m, 4 H), 3.79 (m, 5 H), 4.6 (d, 2 H), 5.05 (m, 1 H), 6.2 (m, 1 H), 6.3 (m, 1 H), 6.7 (s,1 H), 7.32 (m, 3 H), 7.37 (m, 3 H).

### EXAMPLE 9

### 3-({6-Ethyl-4-[4-(phenylacetyl)piperazin-1-yl]thieno[2,3-d]pyrimidin-2-yl}amino)propan-1-ol

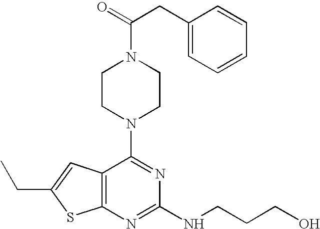

2-Chloro-6-ethyl-4-[4-(phenylacetyl)piperazin-1-yl]thieno[2,3-d]pyrimidine (EXA 6; 0.15 g) and 3-amino-1-propanol (0.084 g) were placed into a 2-dram screw-capped vial and heated at 125° C. overnight. The mixture was then cooled to room temperature add the residue was chromatographed on silica gel using ethyl acetate-hexanes (50/50) as eluent to give 0.164 g of the title compound: MS (ESI+) for C23 H29 N5 O2 S₁ m/z 440.2 (M+H)⁺; ¹H NMR (300 MHz, CDCl₃) δ 1.3 (t, 3 H), 1.74 (m, 2 H), 2.8 (q, 2 H), 3.6 (m, 8 H), 3.8 (m, 7 H), 4.92 (m, 1 H), 6.7 (s, 1 H), 7.26 (m, 3 H), 7.34 (, 2 H);

### EXAMPLE 10

### N-Cyclohexyl-6-ethyl-4-[4-(phenylacetyl)piperazin-1-yl]thieno[2,3-d]pyrimidin-2-amine

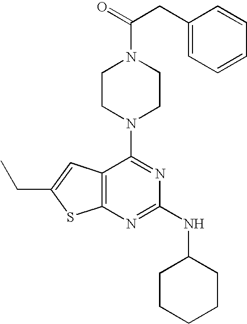

2-Chloro-6-ethyl-4-[4-(phenylacetyl)piperazin-1-yl]thieno[2,3-d]pyrimidine (EXA 6; 0.15 g) and cyclohexylamine (0.112 g) were placed into a 2-dram screw-capped vial and heated at 125° C. overnight. The mixture was then cooled to room temperature and the residue was chromatographed on silica gel using ethyl acetate-hexanes (50/50) as eluent to give 0.0996 g of the title compound: MS (ESI+) for C26 H33 N5 O1 S₁ m/z 464.3 (M+H)⁺; ¹H NMR (300 MHz, CDCl₃) δ 1.2 (m, 4 H), 2.3 (t, 3 H), 1.4 (m, 2 H), 1.75 (m, 2 H), 2.02 (m, 2 H), 2.8 (q, 2 H), 3.58 (s, 4 H), 3.74 (m, 2 H), 3.79 (m, 5 H), 4.68 (d, 1 H), 6.7 (s, 1 H), 7.28 (m, 3 H), 7.36 (m, 2 H).

### EXAMPLE 11

### 2-({6-Ethyl-4-[4-(phenylacetyl)piperazin-1-yl]thieno[2,3-d]pyrimidin-2-yl}amino)ethanol

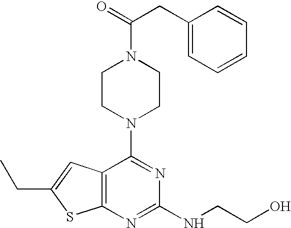

2-Chloro-6-ethyl-4-[4-(phenylacetyl)piperazin-1-yl]thieno[2,3-d]pyrimidine (EXA 6; 0.15 g) and ethanolamine (0.068 g) were placed into a 2-dram screw-capped vial and heated at 125° C. overnight. The mixture was then cooled to room temperature and the residue was chromatographed on silica gel using ethyl acetate-hexanes (I50/50) as eluent to give 0.0916 g of the title compound: MS (ESI+) for C22 H27 N5 O2 S₁ m/z 426.2 (M+H)⁺; ¹H NMR (300 MHz, CDCl₃) δ 1.3 (t, 3 H), 2.8 (q, 2 H), 3.6 (m, 6 H), 3.8 (m, 8 H), 5.15 (m, 1 H), 6.7 (s, 1 H), 7.28 (m, 3 H), 7.37 (m, 2 H).

### EXAMPLE 12

### N-Cyclopentyl-6-ethyl-4-[4-(phenylacetyl)piperazin-1-yl]thieno[2,3-d]pyrimidin-2-amine

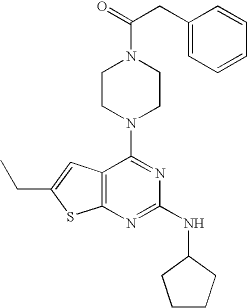

2-Chloro-6-ethyl-4-[4-(phenylacetyl)piperazin-1-yl]thieno[2,3-d]pyrimidine (EXA 6; 0.15 g) and cyclopentylamine (0.1 g) were placed into a 2-dram screw-capped vial and heated at 125° C. overnight. The mixture was then cooled to room temperature and the residue was chromatographed on silica gel using ethyl acetate-hexanes (50/50) as eluent to give 0.108 g of the title compound: MS (ESI+) for C25 H31 N5 O1 S₁ m/z 450.3 (M+H)⁺; ¹H NMR (300 MHz, CDCl₃) δ 1.28 (t, 3 H), 1.45 (m, 2 H), 1.62 (m, 4 H), 2.05 (m, 2 H), 2.8 (q, 2 H), 3.59 (s, 4 H), 3.8 (m, 5 H), 4.24 (m, 1 H), 4.75 (d, 1H), 6.69 (s,1 H), 7.28 (m, 3 H), 7.37 (m, 2 H).

### EXAMPLE 13

### 6-Ethyl-N-(4-fluorobenzyl)-4-[4-(phenylacetyl)piperazin-1-yl]thieno[2,3-d]pyrimidin-2-amine

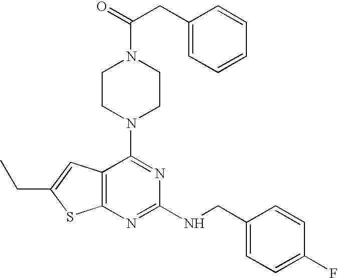

2-Chloro-6-ethyl-4-[4-(phenylacetyl)piperazin-1-yl]thieno[2,3-d]pyrimidine (EXA 6; 0.15 g) and 4-fluorobenzylamine (0.139 g) were placed into a 2-dram screw-capped vial and heated at 110° C. for 8 hours. The mixture was then cooled to room temperature and the residue was chromatographed on silica gel using ethyl acetate-hexanes (50/50) as eluent to give 0.1005 g of the title compound: MS (ESI+) for C27 H28 F1 N5 O1 S₁ m/z 490.2 (M+H)⁺; ¹H NMR (300 MHz, CDCl₃) δ 1.29 (t, 3 H), 2.78 (q, 2 H), 3.53 (m, 4 H), 3.76 (m, 6 H), 4.47 (d, 2 H), 5.07 (m, 1 H), 6.7 (s, 1 H), 6.97 (m, 2 H), 7.32 (m, 7 H).

### EXAMPLE 14

### 6-Ethyl-N-(3-fluorobenzyl)-4-[4-(phenylacetyl)piperazin-1-yl]thieno[2,3-d]pyrimidin-2-amine

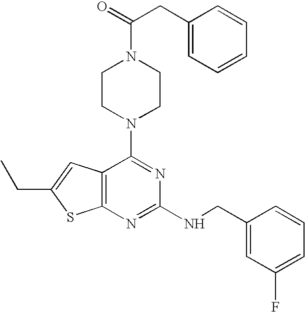

2-Chloro-6-ethyl-4-[4-(phenylacetyl)piperazin-1-yl]thieno[2,3-d]pyrimidine (EXA 6; 0.15 g) and 3-fluorobenzylamine (0.139 g) were placed into a 2-dram screw-capped vial and heated at 110° C. for 8 hours. The mixture was then cooled to room temperature and the residue was chromatographed on silica gel using ethyl acetate-hexanes (50/50) as eluent to give 0.0776 g of the title compound: MS (ESI+) for C27 H28 F1 N5 O1 S₁ m/z 490.2 (M+H)⁺; ¹H NMR (300 MHz, CDCl₃) δ 1.26 (t, 3 H), 2.79 (q, 2 H), 3.53 (m, 4 H), 3.73 (s, 4 H), 3.77 (s, 2 H), 4.6 (d, 2 H), 5.17 (m, 1 H), 6.71 (s, 1 H), 6.92 (t, 1 H), 7.06 (m, 2 H), 7.26 (m, 4 H), 7.34 (m, 2 H).

### EXAMPLE 15

### N-benzyl-6-ethyl-4-[4-(phenylacetyl)piperazin-1-yl]thieno[2,3-d]pyrimidin-2-amine

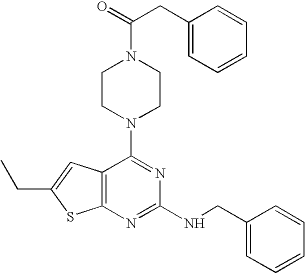

2-Chloro-6-ethyl-4-[4-(phenylacetyl)piperazin-1-yl]thieno[2,3-d]pyrimidine (EXA 6; 0.2 g), 1-butanol (2.0 mL), and benzylamine (0.087 g) were placed into a 2-dram screw-capped vial and placed in a Lab-Line MAX Q2000 orbital shaker at 110° C. for 24 hours. The vial was removed from the orbital shaker and the contents allowed to cool. The solvents were then allowed to evaporate in a vacuum oven warmed at 40° C. To the residue was added THF (10 mL), isocyanate resin (1.1 g; Aldrich, loading 1.84 mmol NCO/g), and tetraalkylammonium carbonate resin (0.7 g; Aldrich, loading 2.86 mmol/g). The vial was then shaken on a shaker block at room temperature for 2 hours. The mixture was then filtered through diatomaceous earth and decolorizing carbon. The solvent was removed under reduced pressure and the residue was chromatographed on silica gel using ethyl acetate-hexane (50/50) to give 0.101 g of the title compound. MS [m+H] 472.2; ¹H NMR (400 MHz, CDCl₃) δ 1.30 (t, 3H), 2.79 (q, 2H), 3.51 (m, 2H), 3.55 (m, 2H) 3.72 (m, 4H), 3.76 (s, 2H), 4.60 (d, 2H), 5.10 (m, 1H), 6.70 (s, 1H), 7.25 (m, 4H), 7.31 (m, 6H).

### EXAMPLE 16

### tert-Butyl 4-(2-chloro-6-ethylthieno[2,3-d]pyrimidin-4-yl)piperazine-1-carboxylate

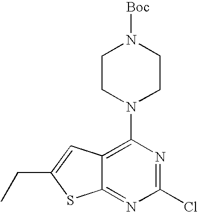

To a mixture of 2,4-dichloro-6-ethylthieno[2,3-d]pyrimidine (EXAM 3; 10.38 g) in dry THF (60 mL) was added diisopropylethylamine (19.4 mL) and Boc-piperazine (9.9 g). The mixture was stirred at room temperature for 6 h, at which time the solvents were removed under reduced pressure and the residue was partitioned between brine and dichloromethane. The layers were separated and the organic layer washed with brine, dried over anhydrous magnesium sulfate and concentrated to dryness to give 15.35 g of the title compound: ¹H NMR (300 MHz, CDCl₃) δ 1.36 (t, 3 H), 1.49 (s, 9 H), 2.89 (q, 2 H), 3.62 (m, 4 H), 3.91 (m, 4 H), 6.95 (s, 1 H).

### EXAMPLE 17

### 2-Chloro-6-ethyl-4-piperazin-1-ylthieno[2,3-d]pyrimidine dihydrochloride

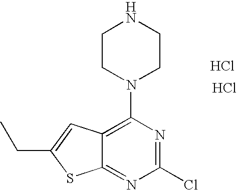

HCl gas was bubbled through dry 1,4-dioxane (400 mL) for 15 minutes. The mixture was cooled to room temperature and tert-butyl 4-(2-chloro-6-ethylthieno[2,3-d]pyrimidin-4-yl)piperazine-1-carboxylate (EXAM 16; 22.1 g) in dry 1,4-dioxane was added. The mixture was stirred at room temperature overnight. The solvent was removed under reduced pressure, dichloromethane was added, and the resulting solids were collected via filtration using to give 19.22 g of the title compound: ¹H NMR 400 MHz, DMSO-d₆) δ 1.28 (t, 3 H), 2.90 (q, 2 H), 3.23 (m, 4 H), 4.05 (m, 4 H), 7.39 (s, 1 H), 9.47 (s, 2 H).

### EXAMPLE 18

### 4-[4-(1,1′-Biphenyl-4-ylcarbonyl)piperazin-1-yl]-2-chloro-6-ethylthieno[2,3-d]pyrimidine

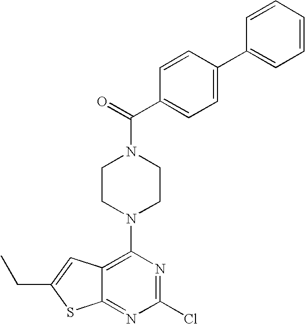

To a mixture of 2-chloro-6-ethyl-4-piperazin-1-ylthieno[2,3-d]pyrimidine dihydrochloride (EXAM 17; 1.02 g) in DMF (5.0 mL) was added diisopropylethylamine (2.0 mL) and 4-biphenyl carbonyl chloride (0.63 g). The mixture was stirred at room temperature for 2 h and then partitioned between ethyl acetate and water. The layers were separated and the organic layer washed four times with brine, dried over anhydrous magnesium sulfate and concentrated. The residue was dissolved in ethyl acetate, adsorbed to silica gel, and placed on top of a ½ inch silica gel plug in a 60 mL sintered glass funnel. The silica was washed with dichloromethane to remove impurities and then eluted with ethyl acetate to give, after concentration, 0.966 g of the title compound: MS (ESI+) for C25 H23 Cl1 N4 O1 S₁ m/z 465.14 (M+H)⁺; ¹H NMR (300 MHz, CDCl₃) δ 1.36 (t, 3 H), 2.9 (q, 2 H), 3.98 (m, 8 H), 6.95 (s, 1 H), 7.37-7.68 (m, 9 H).

### EXAMPLE 19

### tert-Butyl 4-(1,1′-biphenyl-4-ylcarbonyl)piperazine-1-carboxylate

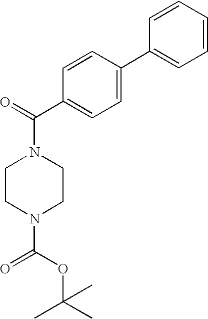

To a mixture of Boc-piperazine (5.0 g) in THF (100 mL) was added 4-biphenyl carbonyl chloride (3.9 g) and diisopropylethylamine (6.0 g). The mixture was stirred at room temperature overnight. The mixture was then partitioned between brine and ethyl acetate. The layers were separated and the organic layer washed with brine, dried over anhydrous magnesium sulfate and concentrated to give 6.0 g of the title compound: ¹H NMR (300 MHz, CDCl₃) δ 1.47 (s, 9 H), 3.4-3.8 (m, 8H), 3.73 (m, 1 H), 7.43-7.48 (m, 4H), 7.57-7.64 (m, 4H).

### EXAMPLE 20

### 1-(1,1′-Biphenyl-4-ylcarbonyl)piperazine hydrochloride

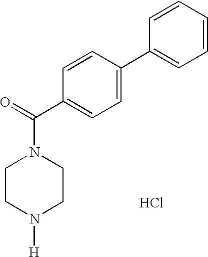

HCl gas was bubbled through methanol (100 mL) for 20 minutes and the solution was cooled to room temperature. tert-Butyl 4-(1,1′-biphenyl-4-ylcarbonyl)piperazine-1-carboxylate (EXAM 18; 6.0 g) was added. The mixture was stirred at room temperature for 20 hours. The solvents were removed under reduced pressure and hexanes were added to the residue. The resulting solids were collected via filtration to give 4.8 g of the title compound: ¹H NMR 400 MHz, DMSO-d₆) δ 3.14 (m, 4 H), 4.14 (m, 4 H), 7.36 (m, 1 H), 7.45 (m, 2 H), 7.54 (m, 2 H), 7.68 (d, 2 H), 7.73 (d, 2 H), 9.64 (s, 1 H).

### EXAMPLE 21

### 2-Chloro-6-ethyl-4-piperazin-1-ylthieno[2,3-d]pyrimidine

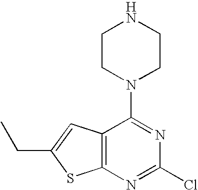

HCl gas was bubbled through a solution of tert-butyl 4-(2-chloro-6-ethylthieno[2,3-d]pyrimidin-4-yl)piperazine-1-carboxylate (EXAM 16; 6.36 g) dissolved in methanol (100 mL) for 1 minute. The mixture was stirred at room temperature for 1 hour. The mixture was then concentrated under reduced pressure. The residue was partitioned between saturated sodium bicarbonate and ethyl acetate. The layers were separated and the organic layer washed with brine, dried over anhydrous magnesium sulfate and concentrated to dryness to give 3.65 g of the title compound: ¹H NMR (400 MHz, CDCl₃) δ 1.34 (t, 3 H), 2.87 (q, 2 H), 3.05 (m, 4 H), 3.96 (m, 4 H), 6.93 (s, 1 H).

### EXAMPLE 22

### 4-[4-(1,1′-Biphenyl-4-ylcarbonyl)piperazin-1-yl]-N-(3,3-diethoxypropyl)-6-ethylthieno[2,3-d]pyrimidin-2-amine

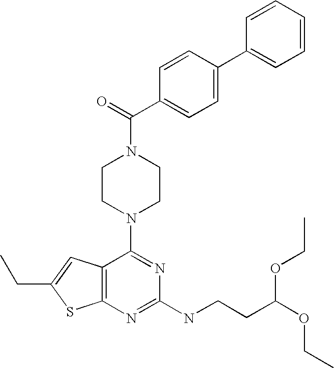

To a mixture of 4-[4-(1,1′-biphenyl-4-ylcarbonyl)piperazin-1-yl]-2-chloro-6-ethylthieno[2,3-d]pyrimidine (EXAM 18; 1.02 g) in 1-butanol (5.0 mL) was added 3-aminopropionaldehyde diethyl acetal (1.07 g). The mixture was placed into a 2-dram screw-cappedped vial. The vial was placed in a 105° C. Lab-Line MAX Q2000 orbital shaker for 18 h, after whichs the contents were cooled and concentrated. The residue was chromatographed on silica gel using methanol-ethyl acetate (5/95) as eluent and then rechromatographed on silica gel using ethyl acetate-hexanes (60/40) to give 0.806 g of the title compound: MS (ESI+) for C32 H39 N5 O3 S₁ m/z574.48 (M+H)⁺. ¹H NMR (400 MHz, CDCl₃) δ 1.20 (t, 6 H), 1.30 (t, 3 H), 1.92 (m, 2 H), 2.79 (q, 2 H), 3.5 (m, 4 H), 3.6-4.0 (m, 10 H), 4.62 (m, 1 H), 5.13 (m, 1 H), 6.73 (s, 1 H), 7.4 (m, 1 H), 7.46 (m, 2 H), 7.51 (m, 2 H), 7.61 (m, 2 H), 7.66 (m, 2 H).

### EXAMPLE 23

### 3-({4-[4-(1,1′-Biphenyl-4-ylcarbonyl)piperazin-1-yl]-6-ethylthieno[2,3-d]pyrimidin-2-yl}amino)propanal

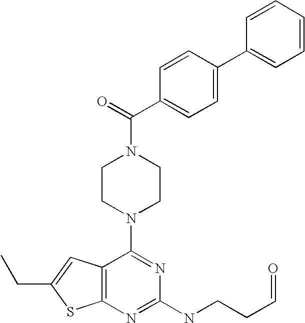

To a mixture of 4-[4-(1,1′-biphenyl-4-ylcarbonyl)piperazin-1-yl]-N-(3,3-diethoxypropyl)-6-ethylthieno[2,3-d]pyrimidin-2-amine (EXAM 22, 0.806 g) in methanol (3 mL) was added water (2 mL) and 4N HCl in dioxane (1 mL). The mixture was heated at 60° C. for 15 minutes at which time the mixture was cooled to room temperature. The mixture was partitioned between saturated sodium bicarbonate and ethyl acetate. The layers were separated and the aqueous layer extracted with ethyl acetate. The ethyl acetate extracts were combined, dried over anhydrous magnesium sulfate, and concentrated to dryness to give 0.544 g of the title compound: ¹H NMR (400 MHz, CDCl₃) δ 1.29 (t, 3 H), 2.79 (q, 2 H), 3.6-4.0 (m, 12 H), 5.06 (m, 1 H), 6.75 (s, 1 H), 7.4 (m, 1 H), 7.46 (m, 2 H), 7.53 (m, 2 H), 7.61 (m, 2 H), 7.66 (m, 2 H), 9.84 (s, 1 H).

### EXAMPLE 24

### N-{4-[4-(1,1′-Biphenyl-4-ylcarbonyl)piperazin-1-yl]-6-ethylthieno[2,3-d]pyrimidin-2-yl}-beta-alanine

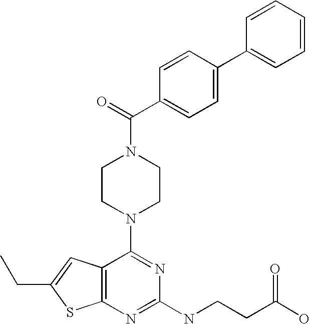

To a mixture of 3-({4-[4-(1,1′-biphenyl-4-ylcarbonyl)piperazin-1-yl]-6-ethylthieno[2,3-d]pyrimidin-2-yl}amino)propanal (EXAM 23; 0.544 g) in dichloromethane (25 mL) was added m-chloroperoxybenzoic acid (0.244 g). The mixture was stirred at room temperature for 4 hours. The mixture was then concentrated and the residue chromatographed on silica gel using MeOH-dichloromethane (5/95) with 0.1% acetic acid as eluent to give 0.323 g of the title compound: MS (ESI+) for C28 H29 N5 O3 S₁ m/z 516.30 (M+H)⁺. ¹H NMR (400 MHz, CDCl₃) δ 1.27 (t, 3 H), 2.61 (m, 2 H), 2.76 (q, 2H), 3.6-4.0 (m, 10 H), 6.73 (s, 1 H), 7.19 (s, 1 H), 7.38 (m, 1 H), 7.46 (m, 2 H), 7.52 (m, 2 H), 7.60 (m, 2 H), 7.65 (m, 2 H).

### EXAMPLE 25

### N³-{4-[4-(1,1′-Biphenyl-4-ylcarbonyl)piperazin-1-yl]-6-ethylthieno[2,3-d]pyrimidin-2-yl}-N˜1˜-[2-hydroxy-1-(hydroxymethyl)ethyl]-beta-alaninamide

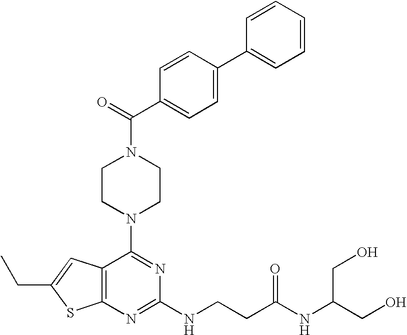

To a mixture of N-{4-[4-(1,1′-biphenyl-4-ylcarbonyl)piperazin-1-yl]-6-ethylthieno[2,3-d]pyrimidin-2-yl}-beta-alanine (EXAM 24; 0.1 g) in NMP (2 mL) was added diisopropylethylamine (0.028 g), serinol (0.019 g), and HATU (0.08 g). The mixture was stirred at room temperature for 4.5 h, at which time the mixture was partitioned between brine and ethyl acetate. The layers were separated and the organic layer washed three times with brine, dried over anhydrous magnesium sulfate and concentrated. The residue was chromatographed on silica gel using methanol-dichloromethane (8/92) to give 0.0842 g of the title compound: MS (ESI+) for C31 H36 N6 O4 S₁ m/z 589.43 (M+H)⁺. ¹H NMR (400 MHz, CDCl₃) δ 1.28 (t, 3 H), 2.52 (m, 2 H), 2.78 (q, 2 H), 3.6-4.0 (m, 15 H), 5.29 (m, 1 H), 6.64 (d, 1 H), 6.74 (s, 1 H), 7.38 (m, 1 H), 7.46 (m, 2 H), 7.51 (m, 2 H), 7.6 (m, 2 H), 7.64 (m, 2 H).

### EXAMPLE 26

### (2S)-3-({4-[4-(1,1′-biphenyl-4-ylcarbonyl)piperazin-1-yl]-6-ethylthieno[2,3-]pyrimidin-2-yl}amino)propane-1,2-diol

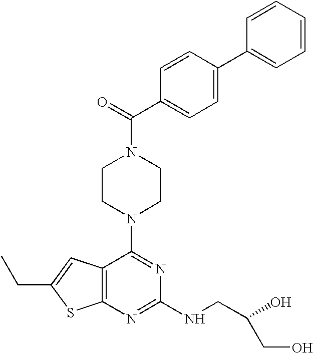

A mixture of 4-[4-(1,1′-biphenyl-4-ylcarbonyl)piperazin-1-yl]-2-chloro-6-ethylthieno[2,3-d]pyrimidine (EXAM 18, 0.150 g), S-(−)-3-amino-1,2-propanediol (0.0885 g), N,N-diisopropylethylamine (0.0461 g), and N-methylpyrrolidinone (1 mL) was stirred at 100° C. for 29 hours. After cooling and standing at room temperature overnight, the mixture was partitioned between ethyl acetate, aq. sodium bicarbonate, and brine. The organic layer was dried over magnesium sulfate, concentrated, and the residue chromatographed on silica gel using MeOH-dichloromethane (6/94) to give 0.100 g of the title compound.

MS [m+H] 518.2; ¹H NMR (400 MHz, CDCl₃) δ 1.30 (t, 3H), 2.79 (q, 2H), 3.5-3.9 (m, 15H), 5.1 (m, 1 H), 6.76 (s, 1 H), 7.35-7.65 (m, 9H).

### EXAMPLE 27

### (2R)-3-({4-[4-(1,1′-biphenyl-4-ylcarbonyl)piperazin-1-yl]-6-ethylthieno[2,3-d]pyrimidin-2-yl}amino)propane-1,2-diol

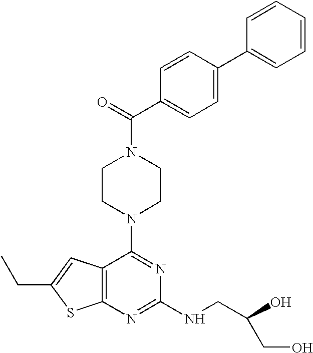

A mixture of 4-[4-(1,1′-biphenyl-4-ylcarbonyl)piperazin-1-yl]-2-chloro-6-ethylthieno[2,3-d]pyrimidine (EXAM 18, 0.150 g), R-(+)-3-amino-1,2-propanediol (0.0885 g), N,N-diisopropylethylamine (0.0461 g), and N-methylpyrrolidinone (1 mL) was stirred at 100° C. for 29 hours. After cooling and standing at room temperature overnight, the mixture was partitioned between ethyl acetate, aq. sodium bicarbonate, and brine. The organic layer was dried over magnesium sulfate, concentrated, and the residue chromatographed on silica gel using MeOH-dichloromethane (6/94) to give 0.0832 g of the title compound. MS [m+H] 518.2; ¹H NMR (400 MHz, CDCl₃) δ 1.30 (t, 3H), 2.79 (q, 2H), 3.5-3.9 (m, 15H), 5.1 (m, 1H), 6.76 (s, 1H), 7.35-7.65 (m, 9H).

### EXAMPLE 28

### N′-{4-[4-(1,1′-biphenyl-4-ylcarbonyl)piperazin-1-yl]-6-ethylthieno[2,3-d]pyrimidin-2-yl}-N,N-dimethylethane-1,2-diamine

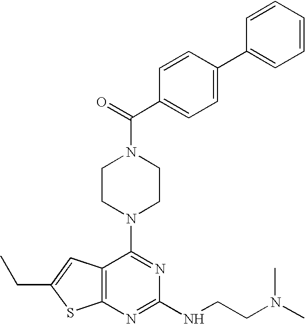

A mixture of 4-[4-(1,1′-biphenyl-4-ylcarbonyl)piperazin-1-yl]-2-chloro-6-ethylthieno[2,3-d]pyrimidine (EXAM 18, 0.150 g), N,N-dimethylethylene diamine (0.73 g), and N-methylpyrrolidinone (0.5 mL) was stirred at 80° C. for 22.5 h, after which an additional 0.07 g of N,N-dimethylethylene diamine was added. After stirring for an additional 32 h, N,N-dimethylethylene diamine (0.07 g) was again added and the mixture was stirred an additional 16 h, after which it was cooled and partitioned between ethyl acetate, aq. sodium bicarbonate, and brine. The organic layer was dried over magnesium sulfate, concentrated, and the residue chromatographed on silica gel using MeOH-dichloromethane (3/97 to 6/94) containing ammonium hydroxide (0.1% by volume) to give 0.042 g of the title compound. MS [m+H] 515.4; ¹H NMR (400 MHz, CDCl₃) δ 1.30 (t, 3H), 2.25 (s, 6H), 2.50 (m, 2H), 2.79 (q, 2H), 3.47 (m, 2H), 3.6-4.0 (m, 8H), 5.24 (m, 1 H), 6.73 (s,1 H), 7.36-7.66 (m, 9H).

### EXAMPLE 29

### (2S)-3-({4-[4-(1,1′-biphenyl-3-ylcarbonyl)piperazin-1-yl]-6-ethylthieno[2,3-d]pyrimidin-2-yl}amino)propane-1,2-diol

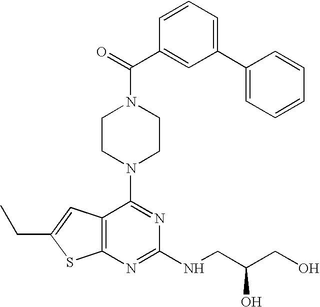

A mixture of 4-[4-(1,1′-biphenyl-3-ylcarbonyl)piperazin-1-yl]-2-chloro-6-ethylthieno[2,3-d]pyrimidine (EXAM 33, 0.101 g), S-(−)-3-amino-1,2-propanediol (0.0595 g), N,N-diisopropylethylamine (0.0338 g), and N-methylpyrrolidinone (1 mL) was stirred at 105° C. for 24 hours. After cooling, the mixture was partitioned between ethyl acetate, aq. sodium bicarbonate, and brine. The organic layer was dried over magnesium sulfate, concentrated, and the residue chromatographed on silica gel using MeOH-ethyl acetate (2/99) to give 0.058 g of the title compound. MS [m+H] 518.3; ¹H NMR (400 MHz, CDCl₃) δ 1.29 (t, 3H), 2.79 (q, 2H), 3.5-4.0 (m, 15H), 5.09 (m, 1H), 6.74 (s, 1H), 7.36-7.7.68 (m, 9H).

### EXAMPLE 30

### (2R)-3-({4-[4-(1,1′-biphenyl-3-ylcarbonyl)piperazin-1-yl]-6-ethylthieno[2,3-d]pyrimidin-2-yl}amino)propane-1,2-diol

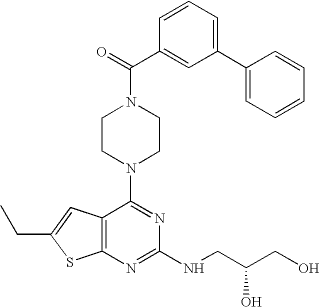

A mixture of 4-[4-(1,1′-biphenyl-3-ylcarbonyl)piperazin-1-yl]-2-chloro-6-ethylthieno[2,3-d]pyrimidine (EXAM 33, 0.101 g), R-(+)-3-amino-1,2-propanediol (0.0595 g), N,N-diisopropylethylamine (0.0338 g), and N-methylpyrrolidinone (1 mL) was stirred at 105° C. for 24 hours. After cooling, the mixture was partitioned between ethyl acetate, aq. sodium bicarbonate, and brine. The organic layer was dried over magnesium sulfate, concentrated, and the residue chromatographed on silica gel using MeOH-ethyl acetate (2/99) to give 0.036 g of the title compound. MS [m+H] 518.3; ¹H NMR (400 MHz, CDCl₃) δ 1.29 (t, 3H), 2.79 (q, 2H), 3.5-4.0 (m, 15H), 5.10 (m, 1H), 6.74 (s, 1H), 7.36-7.68 (m, 9H).

### EXAMPLE 31

### 2-({4-[4-(1,1′-biphenyl-4-ylcarbonyl)piperazin-1-yl]-6-ethylthieno[2,3-d]pyrimidin-2-yl}amino)propane-1,3-diol

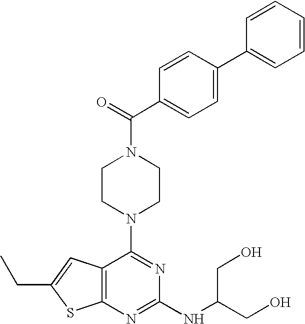

Prepared in the same general manner as for (2S)-3-({4-[4-(1,1′-biphenyl-3-ylcarbonyl)piperazin-1-yl]-6-ethylthieno[2,3-d]pyrimidin-2-yl}amino)propane-1,2-diol [EXA 29]; 4-[4-(1,1′-biphenyl-4-ylcarbonyl)piperazin-1-yl]-2-chloro-6-ethylthieno[2,3-d]pyrimidine (0.125 g) and 2-amino-1,3-propanediol (0.060 g) gave 0.021 g of the title compound. MS [m+H] 518.3; ¹H NMR (400 MHz, CDCl₃) δ 1.30 (t, 3H), 2.80 (q, 2H), 3.6-4.0 (m, 14H), 4.07 (m, 1 H), 5.41 (d, 1H), 6.75 (s, 1H), 7.36-7.66 (9H).

### EXAMPLE 32

### 2-({4-[4-(1,1′-biphenyl-3-ylcarbonyl)piperazin-1-yl]-6-ethylthieno[2,3-d]pyrimidin-2-yl}amino)propane-1,3-diol

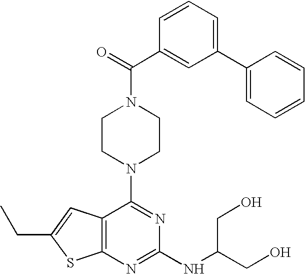

Prepared in the same manner as for (2S)-3-({4-[4-(1,1′-biphenyl-3-ylcarbonyl)piperazin-1-yl]-6-ethylthieno[2,3-d]pyrimidin-2-yl}amino)propane-1,2-diol [EXA 29]; 4-[4-(1,1′-biphenyl-3-ylcarbonyl)piperazin-1-yl]-2-chloro-6-ethylthieno[2,3-d]pyrimidine (0.125 g) and 2-amino-1,3-propanediol (0.060 g) gave 0.032 g of the title compound. MS [m+H] 518.4; ¹H NMR (400 MHz, CDCl₃) δ 1.29 (t, 3H), 2.79 (q, 2H), 3.6-4.0 (m, 14H), 4.06 (m, 1H), 5.39 (d, 1H), 6.74 (s, 1H), 7.38-7.68 (m, 9H).

### EXAMPLE 33

### 4-[4-(1,1′-biphenyl-3-ylcarbonyl)piperazin-1-yl]-2-chloro-6-ethylthieno[2,3-d]pyrimidine

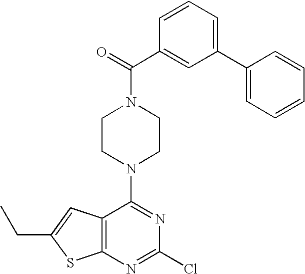

To biphenyl-3-carboxylic acid (0.489 g) in dichloromethane (10 mL) was added 1,1′-carbonyldiimidazole. The mixture was stirred for 45 min, at which time a slurry of 2-chloro-6-ethyl-4-piperazin-1-ylthieno[2,3-d]pyrimidine dihydrochloride (EXAM 17; 0.750 g) and triethylamine (0.33 mL) in dichloromethane (15 mL) and DMF (1 mL) was added. The mixture was stirred overnight and then partitioned between dichloromethane, aqueous sodium bicarbonate, and brine. The organic layer was dried over sodium sulfate and concentrated under reduced pressure. Crystallization from dichloromethane and hexane gave 0.914 g of the title compound. MS [m+H] 463.27; ¹H NMR (400 MHz, CDCl₃) δ 1.34 (3H), 2.88 (2H), 3.6-4.1 (8 H), 6.93 (1H), 7.36-7.53 (5H), 7.58 (2H), 7.67 (2H).

### EXAMPLE 34

### Tert-butyl 4-(2-{[(2R)-2,3-dihydroxypropyl]amino}-6-ethylthieno[2,3-d]pyrimidin-4-yl)piperazine-1-carboxylate

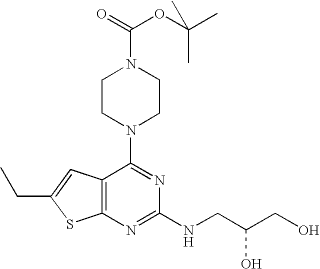

To a mixture of tert-butyl 4-(2-chloro-6-ethylthieno[2,3-d]pyrimidin-4-yl)piperazine-1-carboxylate (EXA 16; 2.0 g) in NMP (5 mL) was added DIEA (1.42 g) and (R)-3-amino-1,2-propanediol (1 g). The mixture was heated at 110° C. for 6 hours, then cooled and stirred at room temperature overnight. The mixture was again heated to 110° C. for 4 hours and cooled to room temperature. The mixture was then partitioned between brine and ethyl acetate. The layers were separated and the aqueous layer extracted with ethyl acetate. The ethyl acetate extracts were combined and washed three times with brine, dried over anhydrous magnesium sulfate and concentrated. The residue was chromatographed on silica gel using ethyl acetate as eluent to give 1.5 g of the title compound: ¹H NMR (400 MHz, CDCl₃) δ 1.3 (t, 3 H), 1.48 (s, 9 H), 2.8 (q, 2 H), 3.6 (m, 8 H), 3.77 (m, 6 H), 5.2 (m, 1 H), 6.8 (s, 1 H).

### EXAMPLE 35

### (2R)-3-[(6-Ethyl-4-piperazin-1-ylthieno[2,3-d]pyrimidin-2-yl)amino]propane-1,2-diol

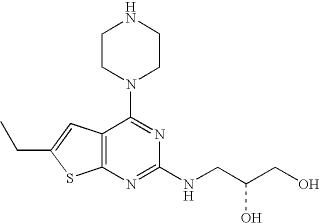

To a mixture of tert-butyl 4-(2-{[(2R)-2,3-dihydroxypropyl]amino}-6-ethylthieno[2,3-d]pyrimidin-4-yl)piperazine-1-carboxylate (EXAM 34; 1.5 g) in methanol (50 mL) was added 4N HCl in dioxane (5 mL). The mixture was stirred at room temperature for 6 h, then placed in a 4° C. refrigerator overnight. The mixture was then partitioned between saturated sodium bicarbonate and ethyl acetate. Solids formed which were not soluble in either layer, thus both layers were concentrated to dryness under reduced pressure. The resulting solids were slurried in ethyl acetate for 10 min and then removed by filtration. The ethyl acetate filtrates were concentrated to dryness to give 0.775 g of the title compound: ¹H NMR (400 MHz, CDCl₃) δ 1.29 (t, 3H), 2.79 (q, 2 H), 2.97 (m, 4 H), 3.61 (m, 4 H), 3.74 (M, 4 H), 3.81 (M, 1 H), 5.14 (M, 1 H), 6.76 (S, 1 H).

### EXAMPLE 36

### (2R)-3-({6-Ethyl-4-[4-(3,3,3-trifluoropropanoyl)piperazin-1-yl]thieno[2,3-d]pyrimidin-2-yl}amino)propane-1,2-diol

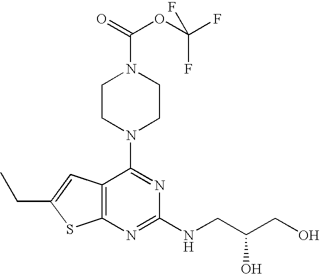

To a mixture of (2R)-3-[(6-ethyl-4-piperazin-1-ylthieno[2,3-d]pyrimidin-2-yl)amino]propane-1,2-diol (EXAM 35, 0.11 g) in NMP (2 mL) was added diisopropylethylamine (0.092 g), 3,3,3-trifluoropropionic acid (0.03 g), and HATU (0.123 g). The mixture was placed in a 2-dram screw-capped vial and placed in a Lab-Line MAX Q2000 orbital shaker at room temperature for 18 hours. The mixture was then partitioned between brine and ethyl acetate. The layers were separated and the organic layer washed three times with brine, dried over anhydrous magnesium sulfate and concentrated. The residue was chromatographed on silica gel using methanol-ethyl acetate (5/95) as eluent to give a solid, which was slurried in ether-hexanes (10/90) and collected to give 0.0442 g of the title compound: MS (ESI+) for C18 H24 F3 N5 O3 S₁ m/z 448.34 (M+H)⁺. ¹H NMR (400 MHz, CDCl₃) δ 1.31 (t, 3 H), 2.82 (m, 3 H), 3.27 (m, 2 H), 3.6 (m, 6 H), 3.8 (m, 6 H), 5.27 (s, 1 H), 6.75 (s, 1 H).

Additional compounds of Formula I that can be prepared in accordance with the synthetic methods of the present invention include those compounds described in Table E.

TABLE E
Formulae

| Example | | |
|---|---|---|
| Number | Structure | Compound Name |
| C-1 | 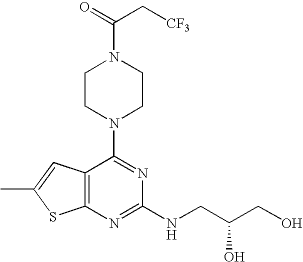 | (2R)-3-({6-methyl-4-[4-(3,3,3-trifluoropropanoyl)piperazin-1-yl]thieno[2,3-d]pyrimidin-2-yl}amino)propane-1,2-diol |
| C-2 | 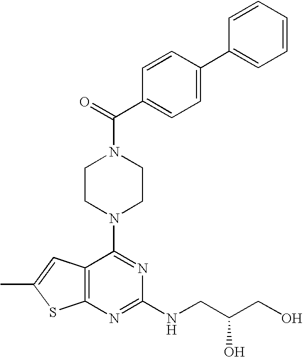 | (2R)-3-({4-[4-(1,1′-biphenyl-4-ylcarbonyl)piperazin-1-yl]-6-methylthieno[2,3-d]pyrimidin-2-yl}amino)propan-1,2-diol |
| C-3 | 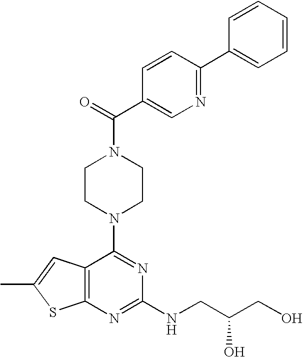 | (2R)-3-[(6-methyl-4-{4-[(6-phenylpyridin-3-yl)carbonyl]piperazin-1-yl}thieno[2,3-d]pyrimidin-2-yl)amino]propan-1,2-diol |
| C-4 | 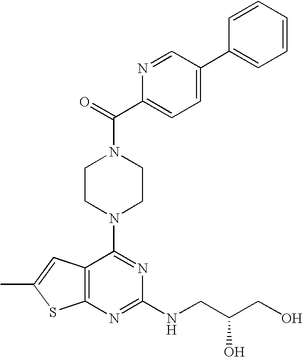 | (2R)-3-[(6-methyl-4-{4-[(5-phenylpyridin-2-yl)carbonyl]piperazin-1-yl}thieno[2,3-d]pyrimidin-2-yl)amino]propane-1,2-diol |
| C-5 | 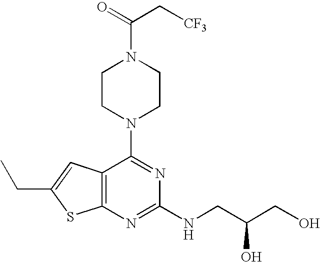 | (2S)-3-({6-ethyl-4-[4-(3,3,3-trifluoropropanoyl)piperazin-1-yl]thieno[2,3-d]pyrimidin-2-yl}amino)propane-1,2-diol |
| C-6 | 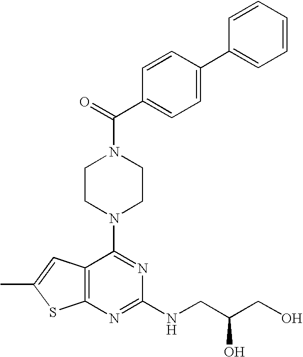 | (2S)-3-({4-[4-(1,1′-biphenyl-4-ylcarbonyl)piperazin-1-yl]-6-methylthieno[2,3-d]pyrimidin-2-yl}amino)propan-1,2-diol |
| C-7 | 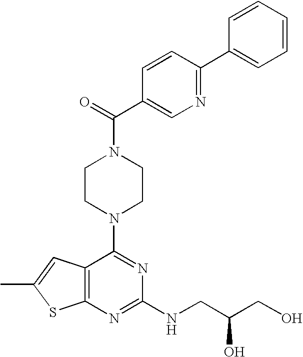 | (2S)-3-[(6-methyl-4-{4-[(6-phenylpyridin-3-yl)carbonyl]piperazin-1-yl}thieno[2,3-d]pyrimidin-2-yl)amino]propan-1,2-diol |
| C-8 | 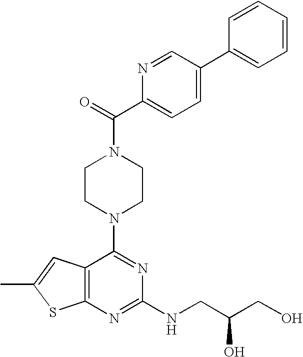 | (2S)-3-[(6-methyl-4-{4-[(5-phenylpyridin-2-yl)carbonyl]piperazin-1-yl}thieno[2,3-d]pyrimidin-2-yl)amino]propane-1,2-diol |
| C-9 | 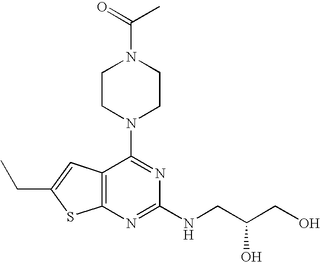 | (2R)-3-{[4-(4-acetylpiperazin-1-yl)-6-ethylthieno[2,3-d]pyrimidin-2-yl]amino}propane-1,2-diol |
| C-10 | 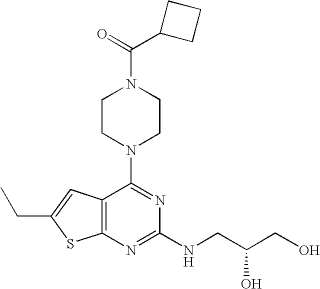 | (2R)-3-({4-[4-(cyclobutylcarbonyl)piperazin-1-yl]-6-ethylthieno[2,3-d]pyrimidin-2-yl}amino)propane-1,2-diol |
| C-11 | 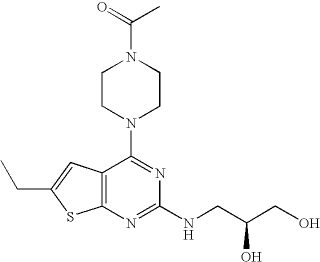 | (2S)-3-{[4-(4-acetylpiperazin-1-yl)-6-ethylthieno[2,3-d]pyrimidin-2-yl]amino}propane-1,2-diol |
| C-12 | 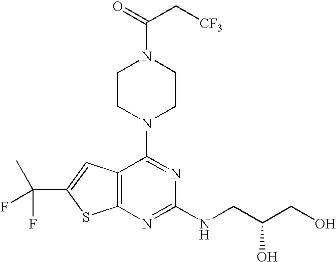 | (2R)-3-({6-(1,1-difluoroethyl)-4-[4-(3,3,3-trifluoropropanoyl)piperazin-1-yl]thieno[2,3-d]pyrimidin-2-yl}amino)propane-1,2-diol |
| C-13 | 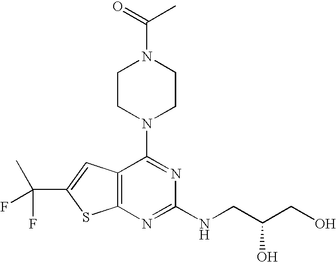 | (2R)-3-{[4-(4-acetylpiperazin-1-yl)-6-(1,1-difluoroethyl)thieno[2,3-d]pyrimidin-2-yl]amino}propane-1,2-diol |
| C-14 | 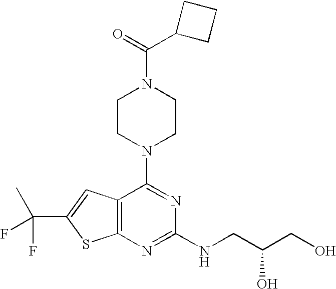 | (2R)-3-{[4-(4-(cylcobutylcarbonyl)piperazin-1-yl]-6-(1,1-difluoroethyl)thieno[2,3-d]pyrimidin-2-yl]amino}propane-1,2-diol |
| C-15 | 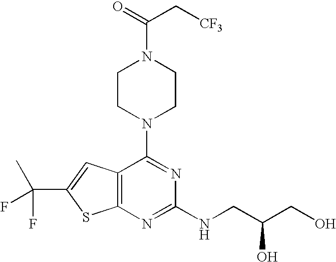 | (2S)-3-({6-(1,1-difluoroethyl)-4-[4-(3,3,3-trifluoropropanoyl)piperazin-1-yl]thieno[2,3-d]pyrimidin-2-yl}amino)propan-1,2-diol |
| C-16 | 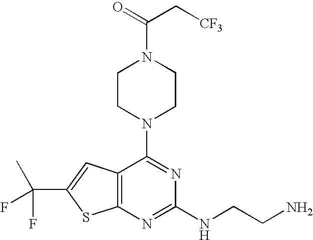 | N-{6-(1,1-difluoroethyl)-4-[4-(3,3,3-trifluoropropanoyl)piperazin-1-yl]thieno[2,3-d]pyrimidin-2-yl}ethane-1,2-diamine |
| C-17 | 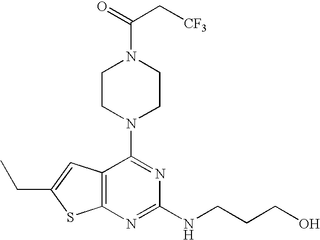 | 3-({6-ethyl-4-[4-(3,3,3-trifluoropropanoyl)piperazin-1-yl]thieno[2,3-d]pyrimidin-2-yl}amino)propan-1-ol |
| C-18 | 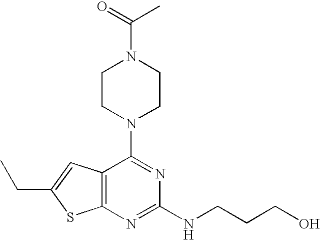 | 3-{[4-(4-acetylpiperazin-1-yl)-6-ethylthieno[2,3-d]pyrimidin-2-yl]amino}propan-1-ol |
| C-19 | 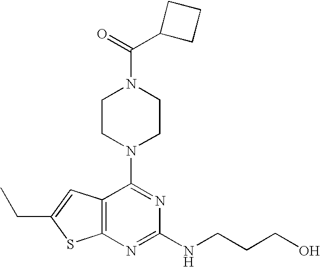 | 3-({4-[4-(cyclobutylcarbonyl)piperazin-1-yl]-6-ethylthieno[2,3-d]pyrimidin-2-yl}amino)propan-1-ol |
| C-20 | 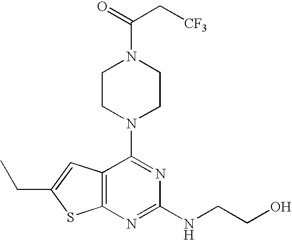 | 2-({6-ethyl-4-[4-(3,3,3-trifluoropropanoyl)piperazin-1-yl]thieno[2,3-d]pyrimidin-2-yl}amino)ethanol |
| C-21 | 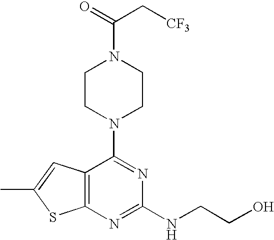 | 2-({6-methyl-4-[4-(3,3,3-trifluoropropanoyl)piperazin-1-yl]thieno[2,3-d]pyrimidin-2-yl}amino)ethanol |
| C-22 | 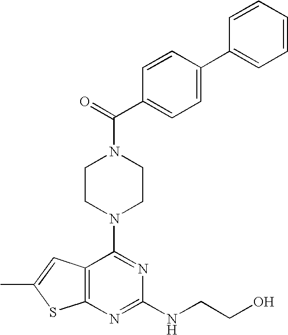 | 2-({4-[4-(1,1′-biphenyl-4-ylcarbonyl)piperazin-1-yl]-6-methylthieno[2,3-d]pyrimidin-2-yl}amino)ethanol |
| C-23 | 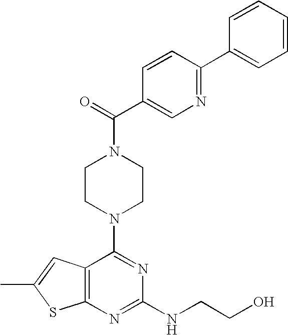 | 2-[(6-methyl-4-{4-[(6-phenylpyridin-3-yl)carbonyl]piperazin-1-yl}thieno[2,3-d]pyrimidin-2-yl)amino]ethanol |
| C-24 | 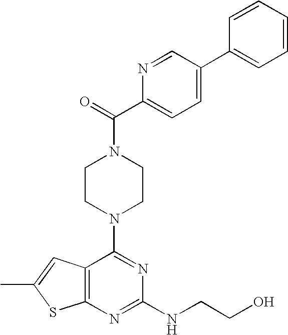 | 2-[(6-methyl-4-{4-[(5-phenylpyridin-2-yl)carbonyl]piperazin-1-yl}thieno[2,3-d]pyrimidin-2-yl)amino]ethanol |
| C-25 | 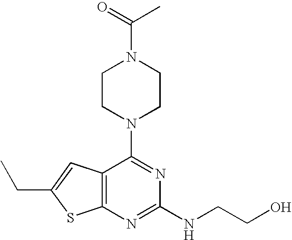 | 2-{[4-(4-acetylpiperazin-1-yl)-6-ethylthieno[2,3-d]pyrimidin-2-yl]amino}ethanol |
| C-26 | 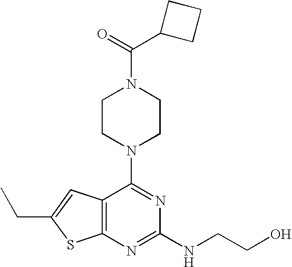 | 2-({4-[4-(cyclobutylcarbonyl)piperazin-1-yl]-6-ethylthieno[2,3-d]pyrimidin-2-yl}amino}ethanol |
| C-27 | 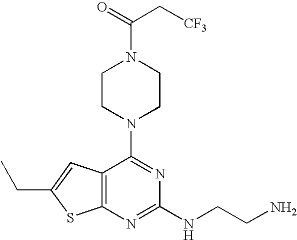 | N-{6-ethyl-4-[4-(3,3,3-trifluoropropanoyl)piperazin-1-yl]thieno[2,3-d]pyrimidin-2-yl}ethane-1,2-diamine |
| C-28 | 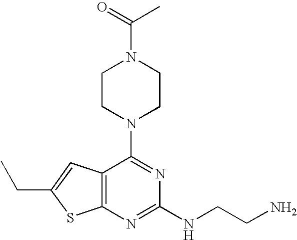 | N-[4-(4-acetylpiperazin-1-yl)-6-ethylthieno[2,3-d]pyrimidin-2-yl]ethane-1,2-diamine |
| C-29 | 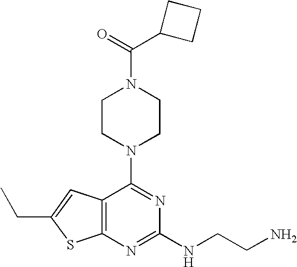 | N-{4-[4-(cyclobutylcarbonyl)piperazin-1-yl]-6-ethylthieno[2,3-d]pyrimidin-2-yl}ethane-1,2-diamine |
| C-30 | 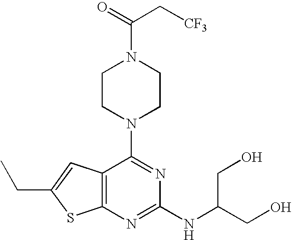 | 2-({6-ethyl-4-[4-(3,3,3-trifluoropropanoyl)piperazin-1-yl]thieno[2,3-d]pyrimidin-2-yl}amino)propane-1,3-diol |
| C-31 | 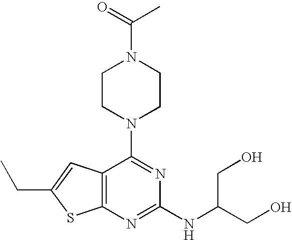 | 2-{[4-(4-acetylpiperazin-1-yl)-6-ethylthieno[2,3-d]pyrimidin-2-yl]amino}propane-1,3-diol |
| C-32 | 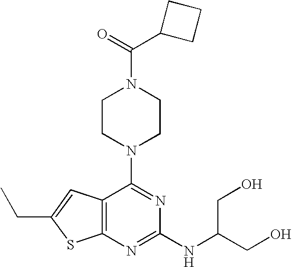 | 2-({4-[4-(cyclobutylcarbonyl)piperazin-1-yl]-6-ethylthieno[2,3-d]pyrimidin-2-yl}amino)propane-1,3-diol |
| C-33 | 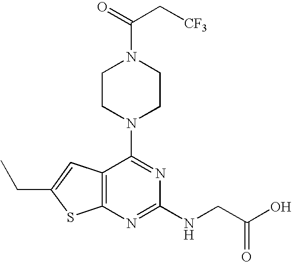 | N-{6-ethyl-4-[4-(3,3,3-trifluoropropanoyl)piperazin-1-yl]thieno[2,3-d]pyrimidin-2-yl}glycine |
| C-34 | 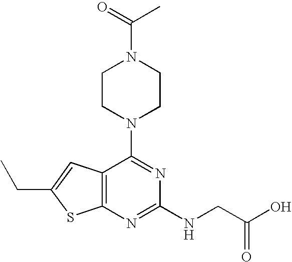 | N-[4-(4-acetylpiperazin-1-yl)-6-ethylthieno[2,3-d]pyrimidin-2-yl]glycine |
| C-35 | 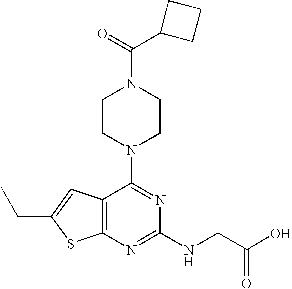 | N-{4-[4-(cyclobutylcarbonyl)piperazin-1-yl]-6-ethylthieno[2,3-d]pyrimidin-2-yl}glycine |
| C-36 | 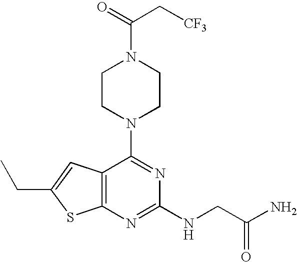 | N²-{6-ethyl-4-[4-(3,3,3-trifluoropropanoyl)piperazin-1-yl]thieno[2,3-d]pyrimidin-2-yl}glycinamide |
| C-37 | 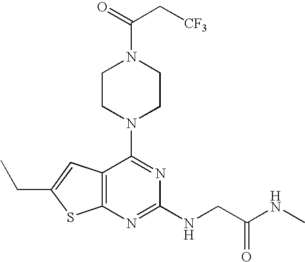 | N²-{6-ethyl-4-[4-(3,3,3-trifluoropropanoyl)piperazin-1-yl]thieno[2,3-d]pyrimidin-2-yl}-N¹-methylglycinamide |
| C-38 | 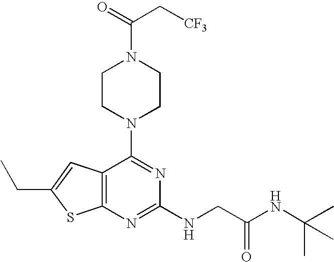 | N¹-(tert-butyl)-N²-{6-ethyl-4-[4-(3,3,3-trifluoropropanoyl)piperazin-1-yl]thieno[2,3-d]pyrimidin-2-yl}glycinamide |
| C-39 | 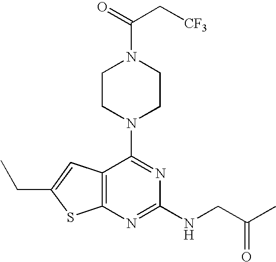 | 1-({6-ethyl-4-[4-(3,3,3-trifluoropropanoyl)piperazin-1-yl]thieno[2,3-d]pyrimidin-2-yl}amino)acetone |
| C-40 | 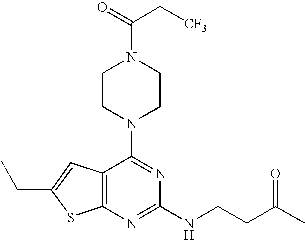 | 4-({6-ethyl-4-[4-(3,3,3-trifluoropropanoyl)piperazin-1-yl]thieno[2,3-d]pyrimidin-2-yl}amino)butan-2-one |
| C-41 | 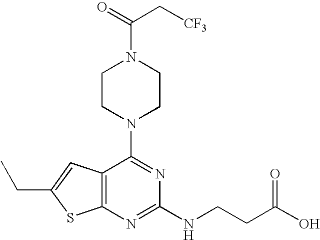 | N-{6-ethyl-4-[4-(3,3,3-trifluoropropanoyl)piperazin-1-yl]thieno[2,3-d]pyrimidin-2-yl}-beta-alanine |
| C-42 | 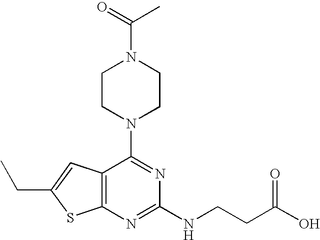 | N-[4-(4-acetylpiperazin-1-yl)-6-ethylthieno[2,3-d]pyrimidin-2-yl]-beta-alanine |
| C-43 | 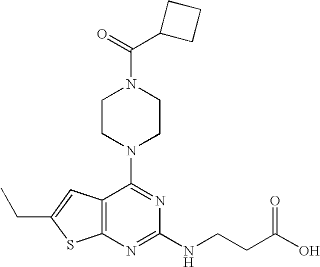 | N-{4-[4-(cyclobutylcarbonyl)piperazin-1-yl]-6-ethylthieno[2,3-d]pyrimidin-2-yl}-beta-alanine |
| C-44 | 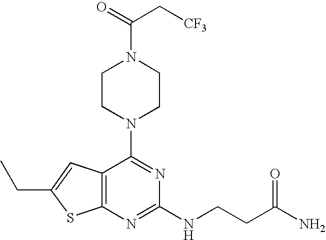 | N³-{6-ethyl-4-[4-(3,3,3-trifluoropropanoyl)piperazin-1-yl]thieno[2,3-d]pyrimidin-2-yl}-beta-alaninamide |
| C-45 | 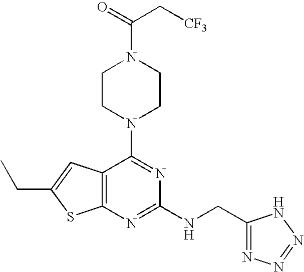 | 6-ethyl-N-(1H-tetrazol-5-ylmethyl)-4-[4-(3,3,3-trifluoropropanoyl)piperazin-1-yl]thieno[2,3-d]pyrimidin-2-amine |
| C-46 | 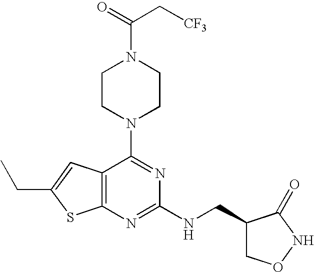 | (4R)-4-[({6-ethyl-4-[4-(3,3,3-trifluoropropanoyl)piperazin-1-yl]thieno[2,3-d]pyrimidin-2-yl}amino)methyl]isoxazolidin-3-one |
| C-47 | 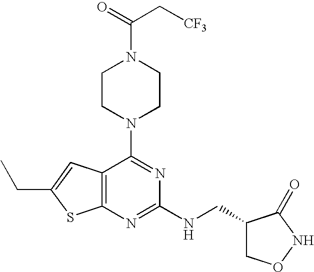 | (4S)-4-[({6-ethyl-4-[4-(3,3,3-trifluoropropanoyl)piperazin-1-yl]thieno[2,3-d]pyrimidin-2-yl}amino)methyl]isoxazolidin-3-one |
| C-48 | 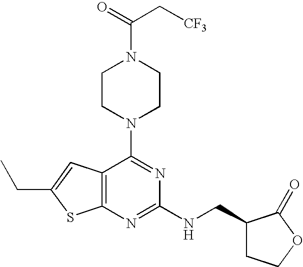 | (3S)-4-[({6-ethyl-4-[4-(3,3,3-trifluoropropanoyl)piperazin-1-yl]thieno[2,3-d]pyrimidin-2-yl}amino)methyl]dihydrofuran-2(3H)-one |
| C-49 | 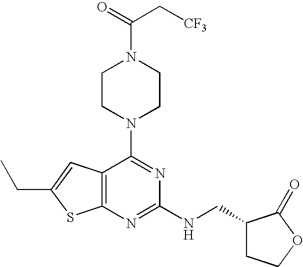 | (3R)-3-[({6-ethyl-4-[4-(3,3,3-trifluoropropanoyl)piperazin-1-yl]thieno[2,3-d]pyrimidin-2-yl}amino)methyl]dihydrofuran-2(3H)-one |
| C-50 | 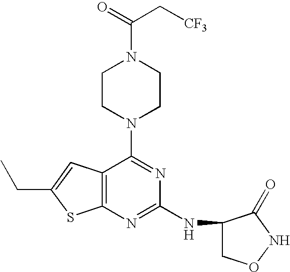 | (4R)-4-({6-ethyl-4-[4-(3,3,3-trifluoropropanoyl)piperazin-1-yl]thieno[2,3-d]pyrimidin-2-yl}amino)isoxazolidin-3-one |
| C-51 | 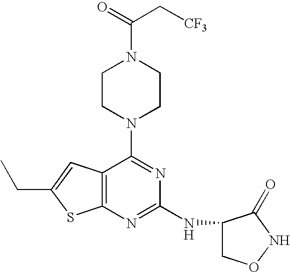 | (4S)-4-({6-ethyl-4-[4-(3,3,3-trifluoropropanoyl)piperazin-1-yl]thieno[2,3-d]pyrimidin-2-yl}amino)isoxazolidin-3-one |
| C-52 | 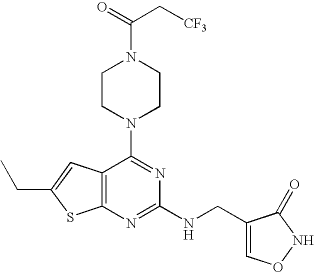 | 4-[({6-ethyl-4-[4-(3,3,3-trifluoropropanoyl)piperazin-1-yl]thieno[2,3-d]pyrimidin-2-yl}amino)methyl]isoxazol-3(2H)-one |
| C-53 | 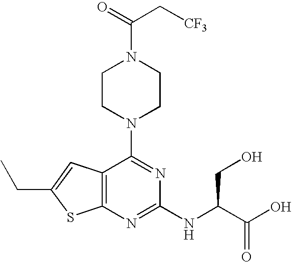 | N-{6-ethyl-4-[4-(3,3,3-trifluoropropanoyl)piperazin-1-yl]thieno[2,3-d]pyrimidin-2-yl}-L-serine |
| C-54 | 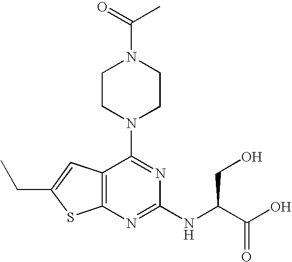 | N-[4-(4-acetylpiperazin-1-yl)-6-ethylthieno[2,3-d]pyrimidin-2-yl]-L-serine |
| C-55 | 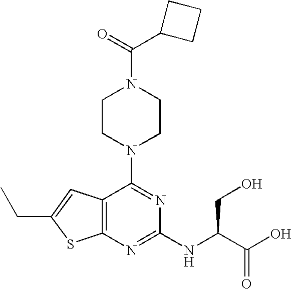 | N-{4-[4-(cyclobutylcarbonyl)piperazin-1-yl]-6-ethylthieno[2,3-d]pyrimidin-2-yl}-L-serine |
| C-56 | 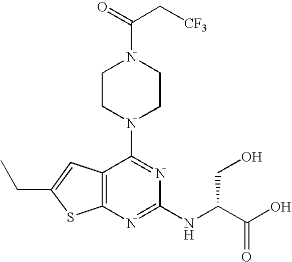 | N-{6-ethyl-4-[4-(3,3,3-trifluoropropanoyl)piperazin-1-yl]thieno[2,3-d]pyrimidin-2-yl}-D-serine |
| C-57 | 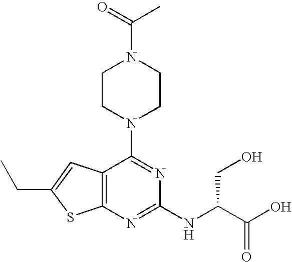 | N-[4-(4-acetylpiperazin-1-yl)-6-ethylthieno[2,3-d]pyrimidin-2-yl]-D-serine |
| C-58 | 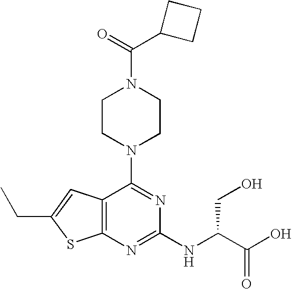 | N-{4-[4-(cyclobutylcarbonyl)piperazin-1-yl]-6-ethylthieno[2,3-d]pyrimidin-2-yl}-D-serine |
| C-59 | 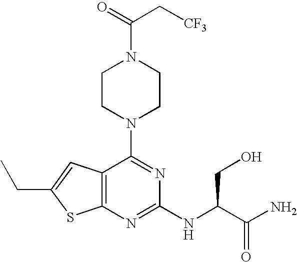 | N²-{6-ethyl-4-[4-(3,3,3-trifluoropropanoyl)piperazin-1-yl]thieno[2,3-d]pyrimidin-2-yl}-L-serinamide |
| C-60 | 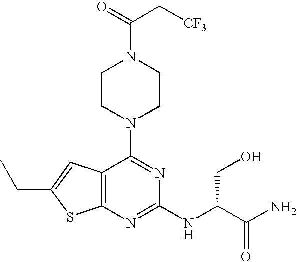 | N²-{6-ethyl-4-[4-(3,3,3-trifluoropropanoyl)piperazin-1-yl]thieno[2,3-d]pyrimidin-2-yl}-D-serinamide |
| C-61 | 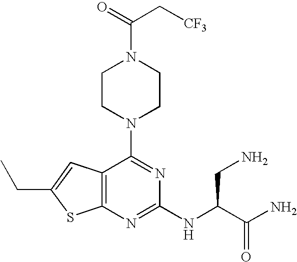 | 3-amino-N²-{6-ethyl-4-[4-(3,3,3-trifluoropropanoyl)piperazin-1-yl]thieno[2,3-d]pyrimidin-2-yl}-L-alaninamide |
| C-62 | 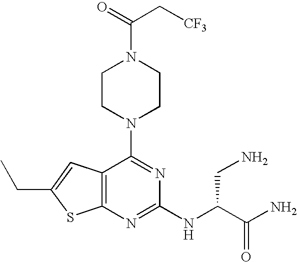 | 3-amino-N²-{6-ethyl-4-[4-(3,3,3-trifluoropropanoyl)piperazin-1-yl]thieno[2,3-d]pyrimidin-2-yl}-D-alaninamide |
| C-63 | 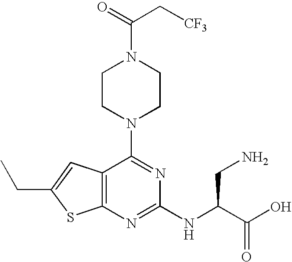 | 3-amino-N-{6-ethyl-4-[4-(3,3,3-trifluoropropanoyl)piperazin-1-yl]thieno[2,3-d]pyrimidin-2-yl}-L-alanine |
| C-64 | 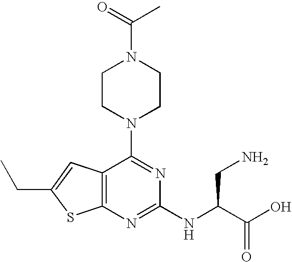 | N-[4-(4-acetylpiperazin-1-yl)-6-ethylthieno[2,3-d]pyrimidin-2-yl]-3-amino-L-alanine |
| C-65 | 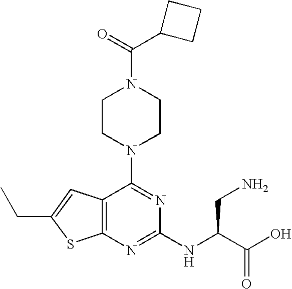 | 3-amino-N-{4-[4-(cyclobutylcarbonyl)piperazin-1-yl]-6-ethylthieno[2,3-d]pyrimidin-2-yl}-L-alanine |
| C-66 | 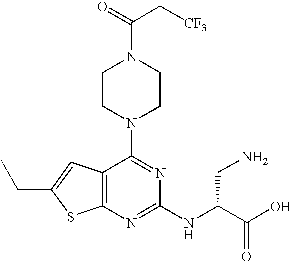 | 3-amino-N-{6-4-[4-(3,3,3-trifluoropropanoyl)piperazin-1-yl]thieno[2,3-d]pyrimidin-2-yl}-D-alanine |
| C-67 | 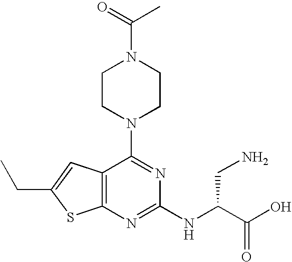 | N-[4-(4-acetylpiperazin-1-yl)-6-ethylthieno[2,3-d]pyrimidin-2-yl]-3-amino-D-alanine |
| C-68 | 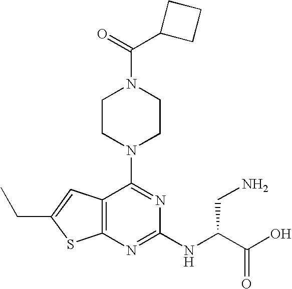 | 3-amino-N-{4-[4-(cyclobutylcarbonyl)piperazin-1-yl]-6-ethylthieno[2,3-d]pyrimidin-2-yl}-D-alanine |

### O. Biological Protocols

### In Vitro Assays

1. Inhibition of [³³P]2MeS-ADP Binding to Washed Human Platelet Membranes. The ability of a test compound to bind to the P2Y12 receptor was evaluated in a platelet membrane binding assay. In this competitive binding assay, the test compound competed against a radiolabelled agonist for binding to the P2Y12 receptor, which is found on the surface of platelets. Inhibition of binding of the labeled material was measured and correlated to the amount and potency of the test compound. Data from this assay are presented in Table F. Platelet rich plasma (“PRP”) was obtained from the Interstate Blood bank, Memphis, Tenn. Platelet rich plasma was prepared from blood units collected in ACD ((prepared by (1) combining: 2.5 g sodium citrate (Sigma S-4641); 1.5 g citric acid (Sigma C-0706); and, 2.0 g dextrose (Sigma G-7528); (2) bringing pH to 4.5; and (3) adding water to bring volume to 100 mL) and using the light spin protocol; this protocol involves centrifugation at room temperature for approximately 20 minutes at speeds up to 160×g. Platelet rich plasma is supplied in units of approximately 200 ml. Each unit is distributed into four 50 mL polypropylene conical tubes for centrifugation, Blood from each donor is maintained separately.

The 50 mL tubes were centrifuged for 15 minutes at 1100 rpm in Sorvall RT6000D (with H1000B rotor). Internal centrifuge temperature was maintained at approximately room temperature (22-24° C.). This spin pelleted cellular components remaining from the PRP preparation. The supernatant was decanted into fresh 50 mL tubes. To avoid carry over of cellular components following the room temperature centrifugation, approximately 5 mL of PRP was left in the tube and discarded. The tubes were capped and inverted 2-3 times and allowed to stand at room temperature for at least 15 minutes following inversion.

Optionally, a Coulter Counter may be used to count platelets from the resting samples during the resting phase. Normal human platelet counts are expected to range from 200,000 to 400,000 per μL of PRP supernatant.

The 50 mL tubes containing PRP supernatant were centrifuged for 15 minutes at 2300-2400 rpm to loosely pellet the platelets. The supernatant from this spin was decanted immediately into a clean cell culture bottle (Corning bottle) and saved in case further centrifugation was needed. The pellet of each tube was resuspended in 2-4 mL of Wash buffer (pH 6.5) (1 L prepared new daily—134 mM NaCl (Sigma S-5150); 3 mM KCl (Sigma P-9333); 1 mM CaCl₂ (JT Baker 1311-01); 2 mM MgCl₂ (Sigma M-2670); 5 mM glucose (EM 4074-2); 0.3 mM NaH₂PO₄ (Sigma S-9638)/12 mM NaHCO₃ (J T Baker 3506-01); 5 mM HEPES pH 7.4 (Gibco 12379-012); 0.35% BSA (Sigma A-7906); 330 mg Heparin (bovine lung, Sigma H-4898); and 30 mL of ACD) by repeated gentle aspiration using disposable polypropylene sample pipettes.

Wash buffer (pH 6.5) was added to each tube to bring the volume to approximately 40 mL. Each tube was incubated for at least 15 minutes at 37° C.

The tubes were then centrifuged again for 15 minutes at 2300-2400 rpm to loosely pellet the platelets. The supernatant was decanted and discarded. The pellet was resuspended in 2-4 mL of Assay buffer (pH 7.4) (1 L volume—134 mM NaCl; 3 mM KCl; 1 mM CaCl₂; 2 mM MgCl₂; 5 mM glucose; 0.3 mM NaH₂PO₄/12 mM NaHCO₃; 5 mM HEPES pH 7.4; and 0.35% BSA) by repeated aspiration using disposable polypropylene sample pipettes. Tubes were combined and gently swirled to mix only when all pellets were successfully resuspended; pellets that did not resuspend or contained aggregates were not combined.

The pooled platelet preparation was counted using a Coulter Counter. The final concentration of platelets was brought to 1×10⁶ per μL using Assay buffer pH 7.4. The platelets were rested for a minimum of 45 minutes at 37° C. before use in the assay.

In one embodiment, the compounds were tested in 96-well microtiter filterplates (Millipore Multiscreen-FB opaque plates, #MAFBNOB50). These plates were used in the assay and pre-wet with 50 μL of Assay buffer pH 7.4 then filtered through completely with a Millipore plate vacuum. Next, 50 μL of platelet suspension was placed into 96-well filterplates. 5 μL of 2MeS-ADP (100 μM working stock concentration to give final concentration 5 μM in well) and 20 μL Assay buffer were added to background control wells. 25 μl Assay buffer were added to set of wells for total binding.

25 μL of 4× concentrated compound were added in duplicate to the 96-well filterplates. Next, 25 μL [³³P]2MeS-ADP (Perkin Elmer NEN custom synthesis, specific activity ˜2100 Ci/mmol) was added to all wells. (1.6 nM working stock concentration to give 0.4 nM final concentration in well). The mixture was incubated for 60 minutes at room temperature and agitated with gentle shaking. The reaction was stopped by washing the 96-well filterplate three times with 100 μl/well of Cold Wash buffer (1 L volume—134 mM NaCl; 10 mM Hepes pH 7.4, stored at 4° C.) on a plate vacuum. The plate was disassembled and allowed to air dry overnight with the filter side up. The filter plates were snapped into adapter plates and 0.1 mL of Microscint 20 Scintillation Fluid (Perkin Elmer # 6013621) was added to each well. The top of the filterplate was sealed with plastic plate covers. The sealed filterplate was agitated for 30 minutes at room temperature. A Packard TopCount Microplate Scintillation Counter was used to measure counts. The binding of compound is expressed as a % binding decrease of the ADP samples after correcting for changes in unaggregated control samples.

### 2. Inhibition of Human Platelet Aggregation

The ability of a test compound to bind to the P2Y12 receptor was evaluated in a platelet aggregation assay. In this functional assay, the test compound competed against an agonist for binding to the P2Y12 receptor, which is found on the surface of platelets. Inhibition of platelet aggregation was measured using standard techniques. Data from this assay are presented in Table F.

As an alternative to the binding assay which measures a candidate compound's ability to bind to the P2Y12 receptor, an assay may be used that measures the effect of the candidate compound on cellular function. The candidate compound competes with ADP, a known agonist, for binding at P2Y12. ADP is sufficient to induce platelet aggregation; the presence of an effective candidate compound inhibits aggregation. The inhibition of platelet aggregation is measured using standard techniques.

Whole blood was collected by Pfizer medical personnel from volunteers (100 mL per volunteer) in 20 mL syringes containing 2 mL of buffered Citrate. In one embodiment, buffered Citrate is 0.105 M Citrate: 0.0840 M Na₃-citrate and 0.0210 M citric acid. In another embodiment, buffered Citrate is 0.109 M Citrate: 0.0945 M Na₃-citrate and 0.0175 M citric acid. The contents of the syringes were expelled into two 50 mL polypropylene conical tubes. Blood was combined only when collected from a single donor. The 50 mL tubes were centrifuged for 15 minutes at 1100 rpm in Sorvall RT6000D (with H1000B rotor). The internal centrifuges temperature was maintained between 22-24° C. and was operating without using the centrifuge brake. This spin pelleted cellular components remaining from the PRP preparation. The PRP layer was collected from each tube and set aside. The supernatant was decanted into fresh 50 mL tubes. To avoid carry over of cellular components following the room temperature centrifugation, approximately 5 mL of PRP was left in the tube and discarded.

The 50 mL tubes were placed back into the centrifuge and spun for 15 minutes at 2800-3000 rpm (with the brake on). This pelleted out most particulate blood constituents remaining, leaving a layer of Platelet Poor Plasma (“PPP”). The PPP was collected and the platelet concentration determined using a Coulter Counter. The PRP layer, previously set aside, was diluted with PPP to a final concentration of approximately 330,000 platelets/μl with the PPP. The final preparation was split into multiple 50 mL conical tubes, each filled with only 25-30 mL of diluted PRP prep. In one embodiment, the tube was filled with 5% CO₂/95% O₂ gas, to maintain the pH of the prep. Each tube was tightly capped and stored at room temperature.

The human platelet aggregation assay is performed is performed in 96-well plate using microtiter plate reader (SpectraMax Plus 384 with SoftMax Pro software from Molecular Devices). The instrument settings include: Absorbance at 626 nm and run time at 15 minutes with readings in 1-minute intervals and 45 seconds shaking between readings.

The reaction is incubated at 37° C. First 18 μl of test compound at 10× final concentration in 5% DMSO is mixed with 144 μl fresh PRP for 30 seconds and incubated at 37° C. for 5 minutes. Following that incubation period, 18 μl of 200 μM ADP is added to the reaction mix. This addition of ADP is sufficient to induce aggregation in the absence of an inhibitor. Results of the assay are expressed as % inhibition, and are calculated using absorbance values at 15 minutes.

3. Human P2Y12 Recombinant Cell Membrane Binding Assay with ³³P 2MeS-ADP.

The ability of a test compound to bind to the P2Y12 receptor was evaluated in a recombinant cell membrane binding assay. In this competitive binding assay, the test compound competed against a radiolabelled agonist for binding to the P2Y12 receptor, expressed on the cell membrane. Inhibition of binding of the labeled material was measured and correlated to the amount and potency of the test compound. Data from this assay are presented in Table F. This binding assay is a modification of the procedure in Takasaki, J. et. al, Mol. Pharmacol., 2001, Vol. 60, pg. 432.

HEK cells were transfected with the pDONR201P2Y12 vector and cultured in MEM with GlutaMAX I, Earle's salts, 25 mM HEPES (Gibco # 42360-032) containing 10% dialyzed FBS (Gibco # 26400-044), 100 μM nonessential amino acids (Gibco # 11140-050), 1 mM sodium pyruvate (Gibco # 11360-070), 0.05% geneticin (Gibco #10131-027), 3 μg/mL blasticidin (Fluka brand from Sigma # 15205), and 0.5 μg/mL puromycin (Sigma # P-8833).

Confluent cells were washed once with cold DPBS (Gibco # 14190-136). Fresh DPBS was added and the cells were scraped and centrifuged at 500×g for 5 minutes at 4° C. The cell pellets were resuspended in TEE buffer (25 mM Tris, 5 mM EDTA, 5 mM EGTA) containing 1 protease inhibitor cocktail tablet (Roche # 1 873 580) per 50 mL (called TEE+Complete) and can be flash frozen at this point.

In one embodiment, frozen cell pellets were used to prepare the membranes. In that embodiment, the frozen cell pellets were thawed on ice. In another embodiment, cell pellets may be used without flash freezing before moving on to the next step.

Cell pellets were resuspended in TEE buffer+Complete and homogenized in a glass dounce for 12 strokes. The cell suspension was centrifuged at 500×g for 5 minutes at 4° C. The supernatant was saved and centrifuged at 20,000×g for 20 minutes at 4° C. This supernatant was discarded and the cell pellet resuspended in TEE buffer+Complete and homogenized in a glass dounce for 12 strokes. This suspension was centrifuged at 20,000×g for 20 minutes at 4° C. and the supernatant discarded. The pellet was resuspended in assay buffer (50 mM Tris, 100 mM NaCl, 1 mM EDTA) containing one protease inhibitor cocktail tablet per 50 mL, and can be flash frozen as 1 mL aliquots at this point.

Dry compounds were diluted as 10 mM DMSO stocks and tested in a seven-point, three-fold dilution series run in triplicate beginning at 10 μM, final concentration. A 1 mM DMSO intermediate stock was made in a dilution plate and from this the seven dilutions were made to 5× the final concentration in assay buffer containing 0.02% BSA.

To a polypropylene assay plate (Costar # 3365) the following were added: a) 30 μL of assay buffer containing one protease inhibitor cocktail tablet per 50 mL; b) 30 μL of 1 nM ³³P 2MeS-ADP made in assay buffer containing 0.02% BSA and 12.5 mg/mL ascorbic acid; 30 μL of cold 1.5 μM 2MeS-ADP for the positive control wells, or assay buffer containing 0.02% BSA and 12.5 mg/mL ascorbic acid for the negative control wells, or 5× drug dilution; and 60 μL of 1 ug/well membranes.

The plates were incubated at room temperature for 1 hour. The reaction was stopped using a cell harvester to transfer the reaction mixture onto GF/B UniFilter plates (Perkin Elmer # 6005177), and washed three times with wash buffer (50 mM Tris), filtering between each wash. The filter plates were dried for approximately 20 minutes in an oven at 50° C. Back seals were adhered to the filter plates and 25 uL of Microscint 20 scintillation fluid (Perkin Elmer # 6013621) were added. The filter plates were sealed, shaken for 30 minutes, and counted on a Top Count. Data were analyzed using a four-parameter curve fit with a fixed minimum and maximum experimentally defined as the average positive and negative controls on each plate, and with a hill slope equal to one.

4. Human P2Y12 Recombinant Cell Membrane Binding Assay with Human Serum Albumin, Alpha-1 Acid Glycoprotein and ³³P 2MeS-ADP

The ability of a test compound to bind to the P2Y12 receptor was evaluated in a recombinant cell membrane binding assay. In this competitive binding assay, the test compound competed against a radiolabelled agonist for binding to the P2Y12 receptor, expressed on the cell membrane. To simulate in vivo conditions, human protein is added to the assay mixture. Inhibition of binding of the labeled material was measured and correlated to the amount and potency of the test compound. Data from this assay are presented in Table F.

HEK cells were transfected with the pDONR²⁰¹P2Y12 vector and cultured in MEM with GlutaMAX I, Earle's salts, 25 mM HEPES (Gibco # 42360-032) containing containing 10% dialyzed FBS (Gibco # 26400-044), 100 μM nonessential amino acids (Gibco # 11140-050), 1 mM sodium pyruvate (Gibco # 11360-070), 0.05% geneticin (Gibco #10131-027), 3 μg/mL blasticidin (Fluka brand from Sigma # 15205), and 0.5 μg/mL puromycin (Sigma # P-8833). Confluent cells were washed once with cold DPBS (Gibco # 14190-136). Fresh DPBS was added and the cells were scraped and centrifuged at 500×g for 5 minutes at 4° C. The cell pellets were resuspended in TEE buffer (25 mM Tris, 5 mM EDTA, 5 mM EGTA) containing 1 protease inhibitor cocktail tablet (Roche # 1 873 580) per 50 mL (called TEE+Complete) and can be flash frozen at this point.

In one embodiment, frozen cell pellets were used to prepare the membranes. In that embodiment, the frozen cell pellets were thawed on ice. In another embodiment, cell pellets may be used without flash freezing before moving on to the next step.

Cell pellets were resuspended in TEE buffer+Complete and homogenized in a glass dounce for 12 strokes. The cell suspension was centrifuged at 500×g for 5 minutes at 4° C. The supernatant was saved and centrifuged at 20,000×g for 20 minutes at 4° C. This supernatant was discarded and the cell pellet resuspended in TEE buffer+Complete and homogenized in a glass dounce for 12 strokes. This suspension was centrifuged at 20,000×g for 20 minutes at 4° C. and the supernatant discarded. The pellet was resuspended in assay buffer (50 mM Tris, 100 mM NaCl, 1 mM EDTA) containing one protease inhibitor cocktail tablet per 50 mL, and can be flash frozen as 1 mL aliquots at this point.

Dry compounds were diluted as 10 mM DMSO stocks and tested in a seven-point, three-fold dilution series run in triplicate beginning at 10 μM, final concentration. A 1 mM DMSO intermediate stock was made in a dilution plate and from this the seven dilutions were made to 5× the final concentration in assay buffer containing 0.02% BSA.

To a polypropylene assay plate (Costar # 3365) the following were added: a) 30 μL of assay buffer containing one protease inhibitor cocktail tablet per 50 mL; b) 30 μL of 1 nM ³³P 2MeS-ADP made in assay buffer containing 0.02% BSA and 12.5 mg/mL ascorbic acid; c) 30 μL of cold 1.5 μM 2MeS-ADP for the positive control wells, or assay buffer containing 0.02% BSA and 12.5 mg/mL ascorbic acid for the negative control wells, or 5× drug dilution; and d) 60 μL of 1 ug/well membranes containing 0.875% human serum albumin (Sigma # A-3782) and 0.0375% alpha-1 acid glycoprotein (Sigma # G-9885).

The plates were incubated at room temperature for 1 hour. The reaction was stopped using a cell harvester to transfer the reaction mixture onto GF/B UniFilter plates (Perkin Elmer # 6005177), and washed three times with wash buffer (50 mM Tris), filtering between each wash. The filter plates were dried for approximately 20 minutes in an oven at 50° C. Back seals were adhered to the filter plates and 25 uL of Microscint 20 scintillation fluid (Perkin Elmer # 6013621) were added. The filter plates were sealed, shaken for 30 minutes, and counted on a Top Count Scintillation Counter.

Data are analyzed using a four-parameter curve fit with a fixed minimum and maximum, experimentally defined as the average positive and negative controls on each plate and with a Hill slope equal to one.

The table below presents the IC₅₀, Kᵢ, and percent inhibition values for compounds tested in either washed human platelets membrane binding assay (assay #1 above) or recombinant cell membrane binding assay (Assay #3, above). Example number refers to the compound prepared as described in the example noted in the section Working Examples, above. The highest concentration of candidate compound tested is listed for each experimental run presented. Multiple data sets indicate multiple experimental runs completed for a given compound.

TABLE F
Data

| | [³³P]2MeS-ADP Binding | | | | [³³P]-2MeS-ADP Binding | | | |
|---|---|---|---|---|---|---|---|---|
| | to Washed Human | | | | to Recombinant Human | | | |
| | Platelet Membranes (Assay 1) | | | | P2Y12 Membranes (Assay 3) | | | |
| Ex. | IC₅₀ | Ki | | [Highest] | IC₅₀ | | | [Highest] |
| # | (μM) | (μM) | % Inhibition | (μM) | (μM) | Ki (μM) | % Inhibition | (μM) |
| 7 | >10 | — | 37.4 | 10 | — | — | — | — |
| 8 | 9.85 | 9.32 | 42.2 | 10 | — | — | — | — |
| 9 | 3.4 | 3.21 | 75 | 10 | — | — | — | — |
| 10 | >10 | — | 37.3 | 10 | — | — | — | — |
| 11 | — | — | — | — | 1.13 | 0.65 | 76.67 | 10 |
| | | | | | 1.19 | 0.687 | 51 | 1 |
| | | | | | 0.905 | 0.905 | 82.93 | 10 |
| 12 | >10 | — | 43.6 | 10 | — | — | — | — |
| 13 | >10 | — | 45.6 | 10 | — | — | — | — |
| 14 | >10 | — | 39.9 | 10 | — | — | — | — |
| 15 | >10 | — | 44 | 10 | — | — | — | — |
| 22 | — | — | — | — | 0.138 | 0.081 | 86 | 10 |
| 24 | — | — | — | — | 0.0132 | 0.008 | 85 | 10 |
| 25 | — | — | — | — | 0.03 | 0.017 | 94 | 10 |
| 26 | — | — | — | — | 0.02 | 0.01 | 98.63 | 10 |
| | | | | | 0.061 | 0.035 | 94.85 | 10 |
| | | | | | 0.047 | 0.027 | 90 | 1 |
| 27 | — | — | — | — | 0.01 | 0.01 | 100.05 | 10 |
| | | | | | 0.048 | 0.028 | 93.86 | 10 |
| | | | | | 0.043 | 0.025 | 92 | 1 |
| 28 | — | — | — | — | 0.044 | 0.027 | 92.3 | 10 |
| | | | | | 0.047 | 0.029 | 98.44 | 10 |
| 29 | — | — | — | — | 0.068 | 0.036 | 97.33 | 10 |
| | | | | | 0.05 | 0.03 | 93.19 | 10 |
| | | | | | 0.132 | 0.076 | 89 | 1 |
| 30 | — | — | — | — | 0.063 | 0.033 | 97.67 | 10 |
| | | | | | 0.08 | 0.05 | 96.44 | 10 |
| | | | | | 0.105 | 0.061 | 88 | 1 |
| 31 | — | — | — | — | 0.021 | 0.012 | 93.05 | 1 |
| 32 | — | — | — | — | 0.096 | 0.051 | 96.45 | 10 |
| 36 | — | — | — | — | 1.05 | 0.557 | 75.7 | 10 |
| | | | | | 1.94 | 1.01 | 70.7 | 10 |

All mentioned documents are incorporated by reference as if here written. When introducing elements of the present invention or the exemplary embodiment(s) thereof, the articles “a,” “an,” “the” and “said” are intended to mean that there are one or more of the elements. The terms “comprising,” “including” and “having” are intended to be inclusive and mean that there may be additional elements other than the listed elements. Although this invention has been described with respect to specific embodiments, the details of these embodiments are not to be construed as limitations.

What is claimed is:

1. A compound, or a pharmaceutically acceptable salt of the compound, wherein the compound has the structure of Formula I: 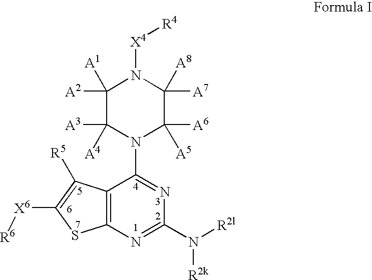
wherein:
A¹, A², A³, A⁴, A⁵, A⁶, A⁷ and A⁸ are independently selected from the group consisting of hydrogen, alkyl, and haloalkyl;
R^{2k} is selected from the group consisting of hydrogen, alkyl, alkenyl, alkynyl, cycloalkyl, aryl and heterocyclyl;
R^{2l} is selected from the group consisting of alkyl, alkenyl, alkynyl, cycloalkyl, aryl and heterocyclyl; wherein:
(a) the R^{2k} and R^{2l} C₇-C₂₀ alkyl, alkenyl, alkynyl, cycloalkyl, aryl and heterocyclyl substituents may be optionally substituted with one or more substituents independently selected from the group consisting of halogen and —R^{2m};
(b) when the R^{2k} substituent is hydrogen and the R^{2l} substituent is C₁-C₆ alkyl, the R^{2l} C₁-C₆ alkyl substituent is substituted with one or more one substituents independently selected from the group consisting of chloro, bromo, iodo and —R^{2m};
(c) when the R^{2k} substituent and R^{2l} substituent are each C₁-C₆ alkyl, the R^{2k} C₁-C₆ alkyl and is substituted with one or more one substituents independently selected from the group consisting of chloro, bromo, iodo and —R^{2m} and the R^{2l} C₁-C₆alkyl may be optionally substituted with one or more substituents independently selected from the group consisting of halogen and —R^{2m};
(d) when the R^{2k} substituent is other than hydrogen and C₁-C₆ alkyl and the R^{2l} substituent is C₁-C₆ alkyl, the R^{2l} C₁-C₆ alkyl substituent may be optionally substituted with one or more substituents independently selected from the group consisting of halogen and —R^{2m};
R^{2m} is selected from the group consisting of oxo, cyano, nitro, amino, alkyl, alkenyl, alkynyl, cycloalkyl, aryl, heterocyclyl, —C(O)R²ⁿ, —C(S)R²ⁿ, —C(O)OR²ⁿ, —C(S)OR²ⁿ, —C(O)SR²ⁿ, —C(O)NR²ⁿR^{2o},
—C(S)NR²ⁿR^{2o}, —OR²ⁿ, —OC(O)R²ⁿ, —OC(S)R²ⁿ, —NR²ⁿR^{2o}, —NR²ⁿC(O)R^{2o}, —NR²ⁿC(S)R^{2o}, —NR²ⁿC(O)OR^{2o},
—NR²ⁿC(S)OR^{2o}, —NR²ⁿS(O)_{R}^{2o}, —NR²ⁿC(O)NR^{2o}R^{2p}, —S(O)_{q}R²ⁿ, —S(O)₂NR²ⁿR^{2o} and —SC(O)R²ⁿ;
q is 0, 1 or 2;
R²ⁿ, R^{2o} and R^{2p} are independently selected from the group consisting of hydrogen, alkyl, alkenyl, alkynyl, cycloalkyl, aryl and heterocyclyl;
wherein the R^{2m}, R²ⁿ, R^{2o} and R^{2p} alkyl, alkenyl, alkynyl, cycloalkyl, aryl, heterocyclyl substituents may be optionally substituted with one or more substituents independently selected from the group consisting of halogen, cyano, nitro, oxo, ═S, —R^{2q}, C(O)R^{2q}, —C(S)R^{2q}, —C(O)OR^{2q}, —C(S)OR^{2q}, —C(O)SR^{2q}, —C(O)NR^{2q}R^{2r}, —C(S)NR^{2q}R^{2r}, —OR^{2q}, —OC(O)R^{2r}, —OC(S)R^{2q}, —NR^{2q}R^{2r}, —NR^{2q}C(O)R^{2r}, —NR^{2q}C(S)R^{2r},
—NR^{2q}C(O)OR^{2r}, —NR^{2q}C(S)OR^{2r}, —NR^{2q}S(O)₂R^{2r}, —NR^{2q}C(O)NR^{2r}R^{2s}, —S(O)ᵣR^{2q}, —S(O)₂NR^{2q}R^{2r} and
—SC(O)R^{2q};
r is 0, 1 or 2;
R^{2q}, R^{2r} and R^{2s} are independently selected from the group consisting of hydrogen, alkyl, alkenyl, alkynyl, cycloalkyl, aryl and heterocyclyl;
wherein the R^{2q}, R^{2r} and R^{2s} alkyl, alkenyl, alkynyl, cycloalkyl, aryl and heterocyclyl substituents may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl;
X⁴ is selected from the group consisting of —C(O)—, —C(S)—, —S(O)— and —S(O)₂—;
R⁴ is selected from the group consisting of —R^{4j}, —OR^{4j}, and —NR^{4j}R^{4k};
wherein R^{4l} and R^{4k} are independently selected from the group consisting of hydrogen, alkyl, alkenyl, alkynyl, cycloalkyl, aryl, heterocyclyl, cycloalkylalkyl, arylalkyl, heterocyclylalkyl, arylcycloalkyl, heterocyclylcycloalkyl, cycloalkylaryl, cycloalkylheterocyclyl, arylaryl, heterocyclylheterocyclyl, arylheterocyclyl, heterocyclylaryl, cycloalkoxyalkyl, heterocyclyloxyalkyl, aryloxyaryl, heterocyclyloxyheterocyclyl, aryloxyheterocyclyl, heterocyclyloxyaryl, arylcarbonylaryl, heterocyclylcarbonylheterocyclyl, aryloxyalkyl, arylcarbonylheterocyclyl, heterocyclylcarbonylaryl, arylcarbonylaminoalkyl, heterocyclylcarbonylaminoalkyl, arylcarbonylaminoalkyl, and heterocyclylcarbonylaminoalkyl;
wherein the R^{4j} and R^{4k} substituents may be optionally substituted with one or more substituents independently selected from the group consisting of halogen, haloalkyl, hydroxyalkyl, oxo, ═S, nitro, cyano, —R^{4l}, —OR^{4l}, —C(O)R^{4l}, —C(O)OR^{4l}, —C(O)NR^{4l}R^{4m}, —OC(O)R^{4l}, —ONR^{4l}R^{4m}, —NR^{4l}R^{4m},
—NR^{4l}C(O)R^{4m}, —NR^{4l}S(O)₂R^{4m}, —S(O)_{b}R^{4l}, —SC(O)R^{4l} and —SC(O)NR^{4l}R^{4m};
b is 0, 1 or 2;
R^{4l} and R^{4m} are independently selected from the group consisting of hydrogen, alkyl, haloalkyl, alkenyl, cycloalkyl, aryl and heterocyclyl;
wherein the R^{4l} and R^{4m} alkyl, haloalkyl, alkenyl, cycloalkyl, aryl and heterocyclyl substituents may be optionally substituted with one or more substituents independently selected from the group consisting of halogen, hydroxy, cyano, oxo, ═S, nitro, —SH, amino, alkyl, haloalkyl, hydroxyalkyl, carboxy, alkoxy, alkoxycarbonyl and alkylamino;
R⁵ is selected from the group consisting of hydrogen, halogen, alkyl, haloalkyl, alkoxy and haloalkoxy;
X⁶ represents a bond or is —C(O)—; wherein:
(a) when X⁶ is —C(O)—, R⁶ is selected from the group consisting of —R^{6a} and —OR^{6a};
(b) when X⁶ represents a bond, R⁶ is selected from the group consisting of halogen, cyano, —R^{6a}
and —OR^{6a};
R^{6a} is selected from the group consisting of hydrogen, alkyl, cycloalkyl and aryl; and
wherein the R^{6a} alkyl, cycloalkyl and aryl substituent may be optionally substituted with one or more substituents independently selected from the group consisting of halogen, hydroxy, oxo, ═S, cyano, alkyl, haloalkyl, hydroxyalkyl, cycloalkyl, carboxy,aryl and heterocyclyl.

2. A compound, or a pharmaceutically acceptable salt of the compound, wherein the compound has the structure of Formula II: 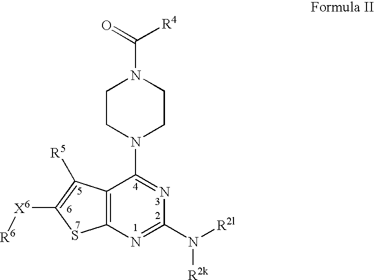
wherein:
R^{2k} is selected from the group consisting of hydrogen, alkyl, alkenyl, alkynyl, cycloalkyl, aryl and heterocyclyl;
R^{2l} is selected from the group consisting of alkyl, alkenyl, alkynyl, cycloalkyl, aryl and heterocyclyl; wherein:
(a) the R^{2k} and R^{2l} alkenyl, alkynyl, cycloalkyl, aryl and heterocyclyl substituents may be optionally substituted with one or more substituents independently selected from the group consisting of halogen and —R^{2m};
(b) when the R^{2k} substituent is hydrogen and the R^{2l} substituent is C₁-C₆ alkyl, the R^{2l} C₁-C₆ alkyl substituent is substituted with one or more one substituents independently selected from the group consisting of chloro, bromo, iodo and —R^{2m};
(c) when the R^{2k} substituent and R^{2l} substituent are each C₁-C₆ alkyl, the R^{2k} C₁-C₆ alkyl and is substituted with one or more one substituents independently selected from the group consisting of chloro, bromo, iodo and —R^{2m} and the R^{2l} C₁-C₆ alkyl may be optionally substituted with one or more substituents independently selected from the group consisting of halogen and —R^{2m};
(d) when the R^{2k} substituent is other than hydrogen and C₁-C₆ alkyl and the R^{2l} substituent is C₁-C₆ alkyl, the R^{2l} C₁-C₆ alkyl substituent may be optionally substituted with one or more substituents independently selected from the group consisting of halogen and —R^{2m};
R^{2m} is selected from the group consisting of cyano, nitro, amino, alkyl, alkenyl, alkynyl, cycloalkyl, aryl, heterocyclyl, —C(O)R²ⁿ, —C(S)R²ⁿ, —C(O)OR²ⁿ, —C(S)OR²ⁿ, —C(O)SR²ⁿ, —C(O)NR²ⁿR^{2o}, —C(S)NR²ⁿR^{2o},
—OR²ⁿ, —OC(O)R²ⁿ, —OC(S)R²ⁿ, —NR²ⁿR^{2o}, —NR²ⁿC(O)R^{2o}, —NR²ⁿC(S)R^{2o}, —NR²ⁿC(O)OR^{2o}, —NR²ⁿC(S)OR^{2o}, —NR²ⁿS(O)₂R^{2o}, —NR²ⁿC(O)NR^{2o}R^{2p}, —S(O)_{q}R²ⁿ, —S(O)₂NR²ⁿR^{2o} and —SC(O)R²ⁿ;
q is 0, 1 or 2;
R²ⁿ, R^{2o} and R^{2p} are independently selected from the group consisting of hydrogen, alkyl, alkenyl, alkynyl, cycloalkyl, aryl and heterocyclyl;
wherein the R^{2m}, R²ⁿ, R^{2o} and R^{2p} alkyl, alkenyl, alkynyl, cycloalkyl, aryl, heterocyclyl substituents may be optionally substituted with one or more substituents independently selected from the group consisting of halogen, cyano, nitro, oxo, =S, —R^{2q}, —C(O)R^{2q}, —C(S)R^{2q}, —C(O)OR^{2q}, —C(S)OR^{2q}, —C(O)SR^{2q}, —C(O)NR^{2q}R^{2r}, —C(S)NR^{2q}R^{2r}, —OR^{2q}, —OC(O)R^{2r}, —OC(S)R^{2q}, —NR^{2q}R^{2r}, —NR^{2q}C(O)^{2r}, —NR^{2q}C(S)R^{2r},
—NR^{2q}C(O)OR^{2r}, —NR^{2q}(S)OR^{2r}, —NR^{2q}S(O)₂R^{2r}, —NR^{2q}C(O)NR^{2r}R^{2s}, —S(O)ᵣR^{2q}, —S(O)₂NR^{2q}R^{2r} and
—SC(O)R^{2q};
r is 0, 1 or 2;
R^{2q}, R^{2r} and R^{2s} are independently selected from the group consisting of hydrogen, alkyl, alkenyl, alkynyl, cycloalkyl, aryl and heterocyclyl;
wherein the R^{2q}, R^{2r} and R^{2s} alkyl, alkenyl, alkynyl, cycloalkyl, aryl and heterocyclyl substituents may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl;
R⁴ is selected from the group consisting of —R^{4j}, —OR^{4j}, and —NR^{4j}R^{4k};
wherein R^{4j} and R^{4k} are independently selected from the group consisting of hydrogen, alkyl, alkenyl, alkynyl, cycloalkyl, aryl, heterocyclyl, cycloalkylalkyl, arylalkyl, heterocyclylalkyl, arylcycloalkyl, heterocyclylcycloalkyl, cycloalkylaryl, cycloalkylheterocyclyl, arylaryl, heterocyclylheterocyclyl, arylheterocyclyl, heterocyclylaryl, cycloalkoxyalkyl, heterocyclyloxyalkyl, aryloxyaryl, heterocyclyloxyheterocyclyl, aryloxyheterocyclyl, heterocyclyloxyaryl, arylcarbonylaryl, heterocyclylcarbonylheterocyclyl, aryloxyalkyl, arylcarbonylheterocyclyl, heterocyclylcarbonylaryl, arylcarbonylaminoalkyl, heterocyclylcarbonylaminoalkyl, arylcarbonylaminoalkyl, and heterocyclylcarbonylaminoalkyl;
wherein the R^{4j} and R^{4k} substituents may be optionally substituted with one or more substituents independently selected from the group consisting of halogen, haloalkyl, hydroxyalkyl, oxo, ═S, nitro, cyano, —R^{4l}, —OR^{4l}, —C(O)R^{4l}, —C(O)OR^{4l}, —C(O)NR^{4l}R^{4m}, —OC(O)R^{4l}, —ONR^{4l}R^{4m}, —NR^{4l}R^{4m},
—NR^{4l}C(O)R^{4m}, —NR^{4l}S(O)₂R^{4m}, —S(O)bR^{4l}, —SC(O)R^{4l} and —SC(O)NR^{4l}R^{4m};
b is 0, 1 or 2;
R^{4l} and R^{4m} are independently selected from the group consisting of hydrogen, alkyl, haloalkyl, alkenyl, cycloalkyl, aryl and heterocyclyl;
R⁵ is selected from the group consisting of hydrogen, halogen, alkyl, haloalkyl, alkoxy and haloalkoxy;
X⁶ represents a bond or is —C(O)—; wherein:
(a) when X⁶ is —C(O)—, R⁶ is selected from the group consisting of —R^{6a} and —OR^{6a};
(b) when X⁶ represents a bond, R⁶ is selected from the group consisting of halogen, cyano, —R^{6a}
and —OR^{6a};
R^{6a} is selected from the group consisting of hydrogen, alkyl, cycloalkyl and aryl; and
wherein the R^{6a} alkyl, cycloalkyl and aryl substituent may be optionally substituted with one or more substituents independently selected from the group consisting of halogen, oxo, ═S, cyano, alkyl, haloalkyl, hydroxyalkyl, cycloalkyl, carboxy, aryl and heterocyclyl.

3. A compound, or a pharmaceutically acceptable salt of the compound, wherein the compound has the structure of Formula III: 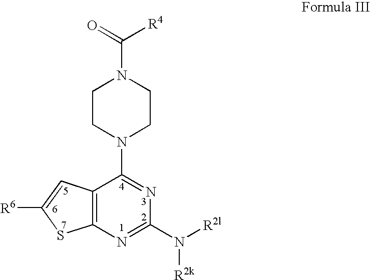
wherein:
R^{2k} is selected from the group consisting of hydrogen, alkyl, alkenyl, alkynyl, cycloalkyl, aryl and heterocyclyl;
R^{2l} is selected from the group consisting of alkyl, alkenyl, alkynyl, cycloalkyl, aryl and heterocyclyl; wherein:
(a) the R^{2k} and R^{2l} alkenyl, alkynyl, cycloalkyl, aryl and heterocyclyl substituents may be optionally substituted with one or more substituents independently selected from the group consisting of halogen and —R^{2m};
(b) when the R^{2k} substituent is hydrogen and the R^{2l} substituent is C₁-C₆ alkyl, the R^{2l} C₁-C₆ alkyl substituent is substituted with one or more one substituents independently selected from the group consisting of chloro, bromo, iodo and —R^{2m};
(c) when the R^{2k} substituent and R^{2l} substituent are each C₁-C₆ alkyl, the R^{2k} C₁-C₆ alkyl and is substituted with one or more one substituents independently selected from the group consisting of chloro, bromo, iodo and —R^{2m} and the R^{2l} C₁-C₆ alkyl may be optionally substituted with one or more substituents independently selected from the group consisting of halogen and —R^{2m};
(d) when the R^{2k} substituent is other than hydrogen and C₁-C₆ alkyl and the R^{2l} substituent is C₁-C₆ alkyl, the R^{2l} C₁-C₆ alkyl substituent may be optionally substituted with one or more substituents independently selected from the group consisting of halogen and —R^{2m};
R^{2m} is selected from the group consisting of cyano, nitro, amino, alkyl, alkenyl, alkynyl, cycloalkyl, aryl, heterocyclyl, —C(O)R²ⁿ, —C(S)R²ⁿ, —C(O)OR²ⁿ, —C(S)OR²ⁿ, —C(O)SR²ⁿ, —C(O)NR²ⁿR^{2o}, —C(S)NR²ⁿR^{2o},
—OR²ⁿ, —OC(O)R²ⁿ, —OC(S)R²ⁿ, —NR²ⁿR^{2o}, —NR²ⁿC(O)R^{2o}, —NR²ⁿC(S)R^{2o}, —NR²ⁿC(O)OR^{2o}, —NR²ⁿC()OR^{2o}, —NR²ⁿS(O)₂R^{2o}, —NR²ⁿC(O)NR^{2o}R^{2p}, —S(O)_{q}R²ⁿ, —S(O)₂NR²ⁿR^{2o} and —SC(O)R²ⁿ;
q is 0, 1 or 2;
R²ⁿ, R^{2o} and R^{2p} are independently selected from the group consisting of hydrogen, alkyl, alkenyl, alkynyl, cycloalkyl, aryl and heterocyclyl;
wherein the R^{2m}, R²ⁿ, R^{2o} and R^{2p} alkyl, alkenyl, alkynyl, cycloalkyl, aryl, heterocyclyl substituents may be optionally substituted with one or more substituents independently selected from the group consisting of halogen, cyano, nitro, oxo, ═S, —R^{2q}, —C(O)R^{2q}, —C(S)R^{2q}, —C(O)OR^{2q}, —C(S)OR^{2q}, —C(O)SR^{2q}, —C(O)NR^{2q}R^{2r}, —C(S)NR^{2q}R^{2r}, —OR^{2q}, —OC(O)R^{2r}, —OC(S)R^{2q}, —NR^{2q}R^{2r}, —NR^{2q}C(O)R^{2r}, —NR^{2q}C()R^{2r},
—NR^{2q}C(O)OR^{2r}, —NR^{2q}C(S)OR^{2r}, —NR^{2q}S(O)₂R^{2r}, —NR^{2q}C(O)NR^{2r}R^{2s}, —S(O)ᵣR^{2q}, —S(O)₂NR^{2q}R^{2r} and
—SC(O)R^{2q};
r is 0, 1 or 2;
R^{2q}, R^{2r} and R^{2s} are independently selected from the group consisting of hydrogen, alkyl, alkenyl, alkynyl, cycloalkyl, aryl and heterocyclyl;
wherein the R^{2q}, R^{2r} and R^{2s} alkyl, alkenyl, alkynyl, cycloalkyl, aryl and heterocyclyl substituents may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl;
R⁴ is selected from the group consisting of —R^{4j}, —OR^{4j}, and —NR^{4j}R^{4k};
wherein R^{4j} and R^{4k} are independently selected from the group consisting of hydrogen, alkyl, alkenyl, alkynyl, cycloalkyl, aryl, heterocyclyl, cycloalkylalkyl, arylalkyl, heterocyclylalkyl, arylcycloalkyl, heterocyclylcycloalkyl, cycloalkylaryl, cycloalkylheterocyclyl, arylaryl, heterocyclylheterocyclyl, arylheterocyclyl, heterocyclylaryl, cycloalkoxyalkyl, heterocyclyloxyalkyl, aryloxyaryl, heterocyclyloxyheterocyclyl, aryloxyheterocyclyl, heterocyclyloxyaryl, arylcarbonylaryl, heterocyclylcarbonylheterocyclyl, aryloxyalkyl, arylcarbonylheterocyclyl, heterocyclylcarbonylaryl, arylcarbonylaminoalkyl, heterocyclylcarbonylaminoalkyl, arylcarbonylaminoalkyl, and heterocyclylcarbonylaminoalkyl;
wherein the R^{4j} and R^{4k} substituents may be optionally substituted with one or more substituents independently selected from the group consisting of halogen, haloalkyl, hydroxyalkyl, oxo, ═S, nitro, cyano, —R^{4l}, —OR^{4l}, —C(O)R^{4l}, —C(O)OR^{4l}, C(O)NR^{4l}R^{4m}, —OC(O)R^{4l}, —ONR^{4l}R^{4m}, —NR^{4l}R^{4m},
—NR^{4l}C(O)R^{4m}, —NR^{4l}S(O)₂R^{4m}, —S(O)_{b}R^{4l}, —SC(O)R^{4l} and —SC(O)NR^{4l}R^{4m};
b is 0, 1 or 2;
R^{4l} and R^{4m} are independently selected from the group consisting of hydrogen, alkyl, haloalkyl, alkenyl, cycloalkyl, aryl and heterocyclyl;
R⁶ is selected from the group consisting of halogen, cyano, —R^{6a} and —OR^{6a};
R^{6a} is selected from the group consisting of hydrogen, alkyl, cycloalkyl and aryl; and
wherein the R^{6a} alkyl, cycloalkyl and aryl substituent may be optionally substituted with one or more substituents independently selected from the group consisting of halogen, oxo, ═S, cyano, alkyl, haloalkyl, hydroxyalkyl, cycloalkyl, carboxy,aryl and heterocyclyl.

4. The compound of claim 3, wherein R⁶ is alkyl, wherein the R⁶ alkyl substituent may be optionally substituted as provided in claim 1.

5. The compound of claim 3, wherein R⁴ is —NR^{4j}R^{4k}; wherein
R^{4j} and R^{4k} are independently selected from the group consisting of hydrogen, alkyl, cycloalkyl and aryl, wherein the R^{4j} and R^{4k} alkyl and aryl may be optionally substituted as provided in claim 1.

6. The compound of claim 3, wherein R⁴ is —R^{4l} or —OR^{4j};
wherein R^{4l} is selected from the group consisting of alkyl, aryl, cycloalkyl, heterocyclyl, arylaryl,
arylalkyl, heterocyclylalkyl, arylcycloalkyl, cycloalkylaryl, arylheterocyclyl, aryloxyaryl, heterocyclyloxyaryl, arylcarbonylaryl, and arylcarbonylaminoalkyl; and wherein the R^{4j} substituents may be optionally substituted as provided in claim 1.

7. The compound of claim 3, wherein R⁴ is —R^{4j} or —OR^{4l};
wherein R^{4j} is selected from the group consisting of methyl, ethyl, propyl, butyl, cyclobutyl, phenyl, fluorenyl, phenylphenyl, phenylmethyl, phenylethyl, phenylphenylmethyl, diphenylethyl, phenyloxymethyl, phenyloxyethyl, phenyloxyphenyl, naphthyloxymethyl, phenylcyclopropyl, phenylcarbonylphenyl, phenylcarbonylaminoethyl, phenylcarbonylaminoethyl, thiophenylmethyl, phenyl-oxadiazolyl, thiazolylphenyl, phenylthiazolyl, phenylpyridinyl, phenylpyrimidinyl, pyridinylphenyl and pyrimidinylphenyl; and wherein the R^{4j} substituents may be optionally substituted as provided in claim 1.

8. A compound, or a pharmaceutically acceptable salt of the compound, wherein the compound has the structure of Formula IV: 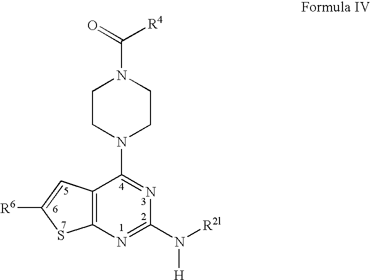
R^{2l} is selected from the group consisting of alkyl, alkenyl, alkynyl, cycloalkyl, aryl and heterocyclyl; wherein:
(a) the R^{2l} C₁-C₂₀ alkyl, alkenyl, alkynyl, cycloalkyl, aryl and heterocyclyl substituents may be optionally substituted with one or more substituents independently selected from the group consisting of halogen and —R^{2m};
(b) the R^{2l} C₁-C₆ alkyl substituent is substituted with one or more one substituents independently selected from the group consisting of chloro, bromo, iodo and —R^{2m};
R^{2m} is selected from the group consisting of cyano, nitro, amino, alkyl, alkenyl, alkynyl, cycloalkyl, aryl, heterocyclyl, —C(O)R²ⁿ, —C(S)R²ⁿ, —C(O)OR²ⁿ, C(S)OR²ⁿ, —C(O)SR²ⁿ, —C(O)NR²ⁿR^{2o}, —C(S)NR²ⁿR^{2o},
—OR²ⁿ, —OC(O)R²ⁿ, —OC(S)R²ⁿ, —NR²ⁿR^{2o}, —NR²ⁿC(O)R^{2o}, —NR²ⁿC(S)R^{2o}, —NR²ⁿC(O)OR^{2o}, —NR²ⁿC(S)OR^{2o}, —NR²ⁿS(O)₂R^{2o}, —NR²ⁿC(O)NR^{2o}R^{2p}, —S(O)_{q}R²ⁿ, —S(O)₂NR²ⁿR^{2o} and —SC(O)R²ⁿ;
q is 0, 1 or 2;
R²ⁿ, R^{2o} and R^{2p} are independently selected from the group consisting of hydrogen, alkyl, alkenyl, alkynyl, cycloalkyl, aryl and heterocyclyl;
wherein the R^{2m}, R²ⁿ, R^{2o} and R^{2p} alkyl, alkenyl, alkynyl, cycloalkyl, aryl, heterocyclyl substituents may be optionally substituted with one or more substituents independently selected from the group consisting of halogen, cyano, nitro, oxo, ═S, —R^{2q}, —C(O)R^{2q}, —C(S)R^{2q}, —C(O)OR^{2q}, —C(S)OR^{2q}, —C(O)NR^{2q}, —C(O)NR^{2q}R^{2r}, —C(S)NR^{2q}R^{2r}, —OR^{2q}, —OC(O)R^{2r}, —OC(S)R^{2q}, —NR^{2q}R^{2r}, —NR^{2q}C(O)R^{2r}, —NR^{2q}C(S)R^{2r},
—NR^{2q}C(O)OR^{2r}, —NR^{2q}C(S)OR^{2r}, —NR^{2q}S(O)₂R^{2r}, —NR^{2q}C(O)NR^{2r}R^{2s}, —S(O)ᵣR^{2q}, —S(O)₂NR^{2q}R^{2r} and
—SC(O)R^{2q};
r is 0, 1 or 2;
R^{2q}, R^{2r} and R^{2s} are independently selected from the group consisting of hydrogen, alkyl, alkenyl, alkynyl, cycloalkyl, aryl and heterocyclyl;
wherein the R^{2q}, R^{2r} and R^{2s} alkyl, alkenyl, alkynyl, cycloalkyl, aryl and heterocyclyl substituents may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl;
R⁴ is selected from the group consisting of —R^{4j}, —OR^{4j}, and —NR^{4j}R^{4k};
wherein R^{4j} and R^{4k} are independently selected from the group consisting of hydrogen, alkyl, alkenyl, alkynyl, cycloalkyl, aryl, heterocyclyl, cycloalkylalkyl, arylalkyl, heterocyclylalkyl, arylcycloalkyl, heterocyclylcycloalkyl, cycloalkylaryl, cycloalkylheterocyclyl, arylaryl, heterocyclylheterocyclyl, arylheterocyclyl, heterocyclylaryl, cycloalkoxyalkyl, heterocyclyloxyalkyl, aryloxyaryl, heterocyclyloxyheterocyclyl, aryloxyheterocyclyl, heterocyclyloxyaryl, arylcarbonylaryl, heterocyclylcarbonylheterocyclyl, aryloxyalkyl, arylcarbonylheterocyclyl, heterocyclylcarbonylaryl, arylcarbonylaminoalkyl, heterocyclylcarbonylaminoalkyl, arylcarbonylaminoalkyl, and heterocyclylcarbonylaminoalkyl;
wherein the R^{4j} and R^{4k} substituents may be optionally substituted with one or more substituents independently selected from the group consisting of halogen, haloalkyl, hydroxyalkyl, oxo, ═S, nitro, cyano, —R^{4l}, —OR^{4l}, —C(O)R^{4l}, —C(O)OR^{4l}, —C(O)NR^{4l}R^{4m}, —OC(O)R^{4l}, —ONR^{4l}R^{4m}, —NR^{4l}R^{4m},
—NR^{4l}C(O)R^{4m}, —NR^{4l}S(O)₂R^{4m}, —S(O)_{b}R^{4l}, —SC(O)R^{4l} and —SC(O)NR^{4l}R^{4m};
b is 0, 1 or 2;
R^{4l} and R^{4m} are independently selected from the group consisting of hydrogen, alkyl, haloalkyl, alkenyl, cycloalkyl, aryl and heterocyclyl;
R⁶ is selected from the group consisting of halogen, cyano, —R^{6a} and —OR^{6a};
R^{6a} is selected from the group consisting of hydrogen, alkyl, cycloalkyl and aryl; and
wherein the R^{6a} alkyl, cycloalkyl and aryl substituent may be optionally substituted with one or more substituents independently selected from the group consisting of halogen, oxo, ═S, cyano, alkyl, haloalkyl, hydroxyalkyl, cycloalkyl, carboxy,aryl and heterocyclyl.

9. The compound of claim 8, wherein R^{2l} is cycloalkyl; wherein the R^{2l} cycloalkyl substituent may be optionally substituted with one or more substituents independently selected from the group consisting of halogen and —R^{2m}; and
R^{2m} is selected from the group consisting of oxo, alkyl, cycloalkyl, aryl, heterocyclyl, —C(O)OR²ⁿ,
—C(O)N R²ⁿR^{2o}, —OR²ⁿ, and —NR²ⁿR^{2o};
R²ⁿ and R^{2o} are independently selected from the group consisting of hydrogen, alkyl, cycloalkyl, aryl and heterocyclyl;
wherein the R^{2m}, R²ⁿ and R^{2o} alkyl, cycloalkyl, aryl, heterocyclyl substituents may be optionally substituted as provided in claim 1.

10. The compound of claim 8, wherein R^{2l} is heterocyclyl; wherein the R^{2l} heterocyclyl substituent may be optionally substituted with one or more substituents independently selected from the group consisting of halogen and —R^{2m};
R^{2m} is selected from the group consisting of oxo, cyano, nitro, amino, —SR²ⁿ, alkyl, aryl, heterocyclyl, —C(O)OR²ⁿ, —C(O)NR²ⁿR^{2o}, —OR²ⁿ, and —NR²ⁿR^{2o};
R²ⁿ and R^{2o} are independently selected from the group consisting of hydrogen, alkyl, cycloalkyl, aryl and heterocyclyl;
wherein the R^{2m}, R²ⁿ and R^{2o} are alkyl, cycloalkyl, aryl, heterocyclyl substituents may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl.

11. The compound of claim 8, wherein R^{2l} is C₁-C₆ alkyl; wherein the R^{2l} C₁-C₆ alkyl substituent is substituted with one or more one substituents independently selected from the group consisting of chloro, bromo, iodo and —R^{2m};
R^{2m} is selected from the group consisting of oxo, cyano, nitro, amino, alkyl, cycloalkyl, aryl, heterocyclyl, —C(O)R²ⁿ, —C(S)R²ⁿ, —C(O)OR²ⁿ, —C(S)OR²ⁿ, —C(O)SR²ⁿ, —C(O)NR²ⁿR^{2o}, —C(S)NR²ⁿR^{2o},
—OR²ⁿ, —OC(O)R²ⁿ, —OC(S)R²ⁿ, —OC(O)OR²ⁿ, —OC(O)NR²ⁿR^{2o}, —OC(S)NR²ⁿR^{2o}, —NR²ⁿR^{2o}, —NR²ⁿC(O)R^{2o},
—NR²ⁿC(S)R^{2o}, —NR²ⁿC(O)OR^{2o}, —NR²ⁿC(S)OR^{2o}, —NR²ⁿS(O)₂R^{2o}, —NR²ⁿC(O)NR^{2o}R^{2p}, —S(O)_{q}R²ⁿ, —S(O)₂NR²ⁿR^{2o} and —SC(O)R²ⁿ;
q is 0, 1 or 2;
R²ⁿ, R^{2o} and R^{2p} are independently selected from the group consisting of hydrogen, alkyl, cycloalkyl, aryl and heterocyclyl;
wherein the R^{2m}, R²ⁿ, R^{2o} and R^{2p} alkyl, cycloalkyl, aryl, heterocyclyl substituents may be optionally substituted as provided in claim 1.

12. The compound of claim 8;
wherein R^{2l} is selected from the group consisting of cycloalkyl, arylalkyl, heterocyclylalkyl, hydroxyalkyl, haloarylalkyl, alkoxyalkyl, oxoalkyl, carboxyalkyl, hydroxyalkylaminocarbonylalkyl and alkylaminoalkyl;
wherein the R^{2l} substituents may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl;
R⁴ is —R^{4j}; wherein R^{4j} is phenylphenyl;
wherein the R^{4j} phenylphenyl each may be optionally substituted with one or more substituents independently selected from the group consisting of oxo, cyano, halogen, alkyl, phenyl, alkoxy, haloalkyl, haloalkoxy, alkylamino, carboxy, alkoxycarbonyl, aminocarbonyl, trifluoromethyl and trifluoromethylmethyl; and R⁶ is selected from the group consisting of selected from the group consisting of halogen, cyano and alkyl.

13. The compound of claim 8;
wherein R^{2l} is selected from the group consisting of phenylpropyl, furanylmethyl, hydroxypropyl, cyclohexyl, hydroxyethyl, cyclopentyl, fluorophenylmethyl, ethoxypropyl, oxopropyl, carboxyethyl, hydroxyethylaminocarbonylethyl, phenylmethyl, hydroxypropyl, methylaminoethyl and hydroxyethylmethyl;
wherein the R^{2l} substituents may be optionally substituted with one or more substituents independently selected from the group consisting of halogen, hydroxy, cyano, oxo, ═S, —SH, nitro, alkyl, haloalkyl, hydroxyalkyl, carboxy, alkoxy and alkoxycarbonyl;
R⁴ is —R^{4j};
wherein R^{4j} is selected from the group consisting of R^{4j} is selected from the group consisting of methyl, ethyl propyl, butyl, cyclopropyl, cyclobutyl, phenyl, fluorenyl, phenylphenyl, phenylmethyl, phenylethyl, phenylphenylmethyl, diphenylethyl, phenyloxymethyl, phenyloxyethyl, phenyloxyphenyl, naphthyloxymethyl, phenylcyclopropyl, phenylcarbonylphenyl, phenylcarbonylaminoethyl, phenylcarbonylaminoethyl, thiophenylmethyl, phenyl-oxadiazolyl, oxadiazolylphenyl, thiazolylphenyl, phenylthiazolyl, phenylpyridinyl, phenylpyrimidinyl, pyridinylphenyl and pyrimidinylphenyl;
wherein the R^{4j} substituents each may be optionally substituted with one or more substituents independently selected from the group consisting of oxo, cyano, chloro, bromo, fluoro, methyl, ethyl, propyl, butyl, phenyl, methoxy, trifluoromethyl, trifluoromethylmethyl, trifluoromethoxy, ethoxy, propoxy, butoxy, dimethylamino, carboxy, methoxycarbonyl and aminocarbonyl; and
R⁶ is ethyl.

14. A pharmaceutical composition comprising a therapeutically effective amount of a compound of claim 1.

15. A method of treating a platelet dependent thrombosis or a platelet dependent thrombosis-related condition in a subject, comprising administering to the subject a therapeutically effective amount of a compound of claim 1.
